(19) United States
(12) Patent Application Publication
(10) Pub. No.: US 2010/0105145 A1
(43) Pub. Date: Apr. 29, 2010

(54) METHOD OF SIMULTANEOUSLY VISUALIZING MULTIPLE BIOLOGICAL TARGETS

(75) Inventor: Lars Winther, Smorum (DK)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: DAKO DENMARK A/S, Glostrup (DK)

(21) Appl. No.: 11/993,584

(22) PCT Filed: Jun. 30, 2006
(86) PCT No.: PCT/IB2006/003126

§ 371 Date: Jan. 4, 2010

Related U.S. Application Data
(60) Provisional application No. 60/695,410, filed on Jul. 1, 2005.

(51) Int. Cl. G01N 33/68; G01N 21/00; G01N 33/48

(52) U.S. Cl. 436/86; 436/164; 436/94

(57) ABSTRACT

The present invention relates to optionally automated methods that may be used to qualitatively and/or quantitatively detect at least one, or for example, two or more different targets in a sample, and kits associated with such methods. The two or more different targets may be detected and distinguished by adding at least one cross-linking agent to the sample in between different steps of a detection procedure. The addition of a cross-linking agent may allow for drastic changes in buffer conditions (i.e. solvent, pH, salt concentration, etc.) or temperature in order to refine a detection procedure with minimal loss of signal. The instant invention is compatible with a variety of detection systems, including immunohistochemistry (IHC), immunocytochemistry (ICC), in situ hybridization (ISH), flow cytometry, enzyme immuno-assays (EIA), enzyme linked immuno-assays (ELISA), blotting methods (e.g. Western, Southern, and Northern), labeling inside electrophoresis systems or on surfaces or arrays, and precipitation, among other general detection assay formats. The invention is also compatible with many different types of samples, targets, probes, and detectable labels.

The present invention claims priority to U.S. Provisional Application No. 60/695,410, which is incorporated herein by reference.

### DESCRIPTION OF THE INVENTION

The present invention relates to optionally automated methods that may be used to qualitatively and/or quantitatively detect one target in a sample, as well as to detect two or more different targets in a sample, and kits associated with such methods. The two or more different targets may be detected and distinguished by adding at least one cross-linking agent to the sample in between different steps of a detection procedure. The addition of a cross-linking agent may allow for drastic changes in buffer conditions (i.e. solvent, pH, salt concentration, etc.) or temperature in order to refine a detection procedure with minimal loss of signal. The instant invention is compatible with a variety of detection systems, including immunohistochemistry (INC), immunocytochemistry (ICC), in situ hybridization (ISH), flow cytometry, enzyme immuno-assays (EIA), enzyme linked immuno-assays (ELISA), blotting methods (e.g. Western, Southern, and Northern), labeling inside electrophoresis systems or on surfaces or arrays, and precipitation, among other general detection assay formats. The invention is also compatible with many different types of samples, targets, probes, and detectable labels.

Methods of visualizing two or more different targets within the same sample are beneficial in a variety of contexts. For example, within a biological sample, one may wish to assess the expression level of different proteins at different locations within the sample and therefore, to visualize different proteins in the same sample with different detectable labels. Alternatively, one may wish to correlate a particular cellular DNA or RNA sequence in a cell with the presence of one or more different proteins in that same cell, or to detect more than one different type of cellular nucleic acid.

Even when only one target is detected, many samples need to be manipulated, sometimes under harsh conditions, in order to render certain targets available for detection (in other words to retrieve the targets). Methods of target detection can generally be limited, for example, if incompatible buffer conditions are needed during different stages of a detection process. Target retrieval is a delicate process, in which both insufficient or excess target retrieval can reduce the staining below the limit of detection. In some cases, various targets need different target retrieval treatments. For example, one target may be retrieved only at acidic pH and another in the same sample only at basic pH. Some targets may require protease or nuclease treatment, organic solvents, or heat denaturation to become detectable. However, pH changes, heat, organics, and enzyme treatments may destroy other potential targets in a system and may also adversely affect previously applied detectable labels for other targets. Further, even if only one target is detected, the conditions needed for probe binding and for later labeling may be different.

Also, the position of diagnostic targets can change due to many manipulations needed for many detection protocols. For example, a target site may smear or diffuse with repeated handling or over time. In addition, some targets are cross-reactive with probes for other targets.

Some embodiments of the instant invention employ cross-linking agents which may stabilize the detection of one or more targets so that buffer conditions can be altered in later steps to make them compatible with later-used detection reagents or to retrieve and/or detect other targets in the sample, and which may limit the diffusion of a detectable label over time. In some embodiments, the cross-linkers form covalent attachments between the components of the detection system and/or the targets. The cross-linking agents of the invention may increase the range of present target detection methods. They may be used at various points during a given protocol, and may also be reversible in some embodiments.

In some embodiments, the probe is covalently or non-covalently associated with another recognition element that is later detected by a detectable label. For instance, the probe may bind to a target using one type of molecular recognition that is stable in a particular buffer system, but the interaction of the associated recognition element and the detectable label occurs through a different type of molecular recognition event that is stable in a different buffer system that may be incompatible with that used to apply the probe. By cross-linking the probe to the target, one can change the buffer system to one that allows the recognition element to bind to a detectable label with less concern over losing signal from the probe. In effect, the binding language of the probe-target interaction is translated into that of the detectable label-recognition element interaction, each of which operates in a different medium. Thus, when detecting multiple targets, one may apply a series of probes, for example, cross-link at least once during or after application of the probes, and then change the buffer conditions and add the detectable labels.

In some embodiments, probes and detectable labels may recognize each other indirectly through one or more adaptor molecules such as a hapten or an engineered molecular entity. In such a situation, the probe binds to one or more adaptors, which act as recognition elements for the detectable labels. As above, by allowing multiple layers of molecules to form between a probe and a detectable label, and cross-linking components of the detection system in the sample, the buffer system may be switched to one that would ordinarily be incompatible with the target or probe. Adaptor units may also serve to amplify the detection signal from a target, for example, by providing many binding sites for detectable labels such that many detectable labels are associated with each target.

Further, certain detection processes or steps are conducted in aqueous buffers while others require organics. It is desirable to be able to use both types of buffers on one sample. The present invention for example allows one to retrieve or detect a first target in an aqueous medium and then switch to an organic medium to detect or retrieve a second target, or vice versa, without significantly losing the signal from the first target.

Further objects and advantages of the invention will be set forth in part in the description which follows, and in part will be obvious from the description, or may be learned by practice of the invention. It is to be understood that both the foregoing general description and the following more detailed description are exemplary and explanatory only and are not restrictive of the invention, as claimed.

### BRIEF DESCRIPTION OF THE DRAWINGS

FIG. 1 illustrates two examples of how the cross-linker may be employed to stabilize a detection system for a change of buffer conditions. Buffer 1 and 2 represent two different, possibly incompatible buffer conditions, such as different salt concentrations, pH, temperature, and the like, or more drastic differences such as a change from an aqueous medium to an organic medium or vice versa. The cross-linker may be added prior to the change of reaction conditions to stabilize the system. In Example 1a, the cross-linker is added after the first target is probed and labeled, while in Example 1 b, the cross-linker is added after the targets are exposed to probes, but prior to the addition of the detectable labels that will recognize the probes.

FIG. 2 illustrates another reaction scheme compatible with the instant invention, in which the cross-linker is added after de-waxing (de-paraffination) prior to beginning the detection process.

FIG. 3 illustrates another type of system compatible with the invention, which is also further described in U.S. Provisional Application No. 60/695,410, and in accompanying International Applications entitled “Method of Detecting Targets in an IHC (or ISH) Sample,” all of which are incorporated herein by reference. The left panel illustrates an exemplary recognition unit according to the invention, comprising a nucleic acid analog segment (shaded bar), a linker (thin line), a polymer (thick line), and an antibody probe (upside-down Y shape). The center panel illustrates an exemplary optional adaptor unit according to the invention, comprising two nucleic acid analog segments (shaded bars), linkers (thin lines), and a polymer (thick line). The right panel illustrates an exemplary detection unit according to the invention, comprising detectable labels (shaded octagons), polymers (thick lines), a linker (thin line), and a nucleic acid analog segment (shaded bar).

FIG. 4 illustrates an exemplary two-layer method according to the previous system in which a target antigen bound to a primary antibody is recognized by a recognition unit comprising a secondary antibody probe. The recognition unit is specifically hybridized to a detection unit via the nucleic acid analog segments on each unit.

FIG. 5 illustrates an exemplary three-layer method according to the previous system wherein a target antigen bound to a primary antibody is recognized by a recognition unit comprising a secondary antibody probe and a nucleic acid analog segment. The recognition unit specifically hybridizes to an adaptor unit comprising nucleic acid analog segments that specifically hybridize to the recognition unit and a detection unit.

FIG. 6 illustrates a system which may allow for visualization of more than one target in a sample, such as, here: two different proteins and a DNA segment. In this embodiment, three different two-layer systems, each comprising a recognition unit and a detection unit are employed together. Each detection unit carries a different detectable label such that the detectable labels are distinguishable from each other. Each set of recognition unit and detection unit does not cross-react with the other sets or with any other probes or targets in the sample. A cross-linker could be used in conjunction with this sort of system, for example, when the different targets are detected or retrieved under different conditions.

FIG. 7(a-r): Examples of non-natural bases that may be used in the nucleic acid analog segments of the invention, and their names and symbols. Where:

R1 denotes the attachment point to the backbone

R2 is, for example, substituents in the 8-postion of purines: such as hydrogen, halogens, or other small substituents i.e., methyl, ethyl.

R3 is, for example, substituents on hydrogen bonding exocyclic amino groups on bases other than cytosine: such as hydrogen, methyl, ethyl, acetyl.

R4 is, for example, substituents that face a carbonyl in place of an amino group: such as hydrogen, fluorine and chlorine.

R5 is, for example, substituents in the 5-position of pyrimidines: for example, fluorofors, hydrogen, halogens, and substituted and unsubstituted groups of C1-C20. This position, for example, allows bulky substituents, if desired.

R6 is, for example, substituents on the hydrogen bonding excocylic amino group of cytosine. This position also allows bulky substituents, for example, alkyl, acyl, and substituted and unsubstituted groups of C1-C20.

See the accompanying International Application entitled “New Nucleic Acid Base Pairs” for further information on these pairing schemes.

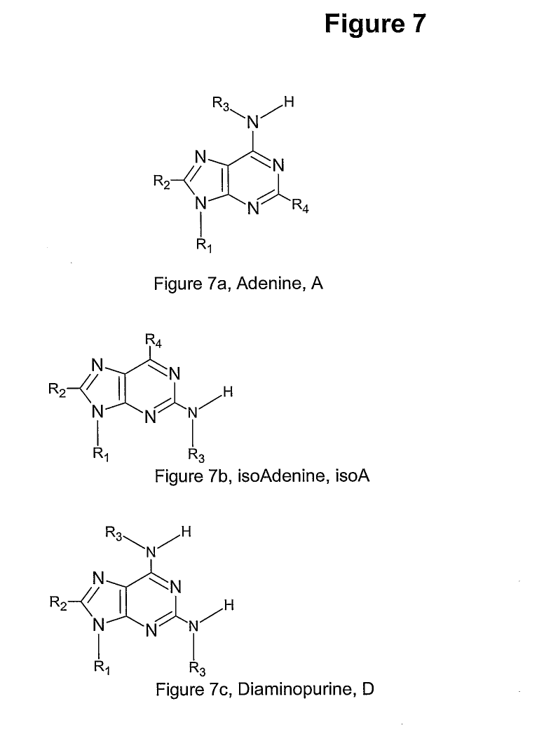

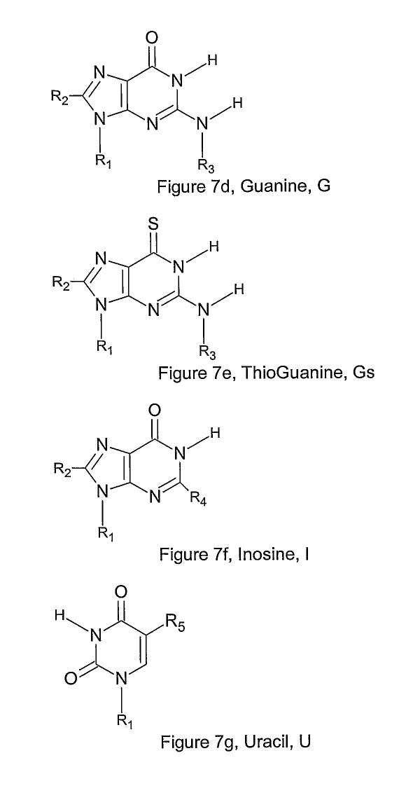

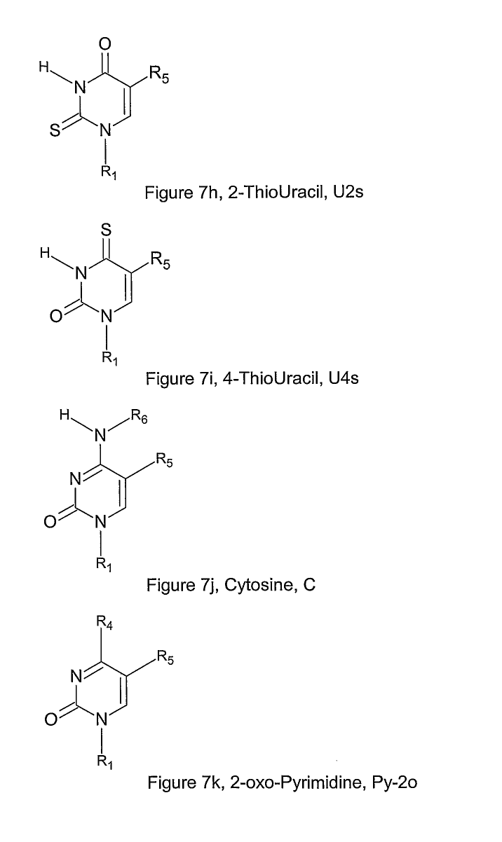

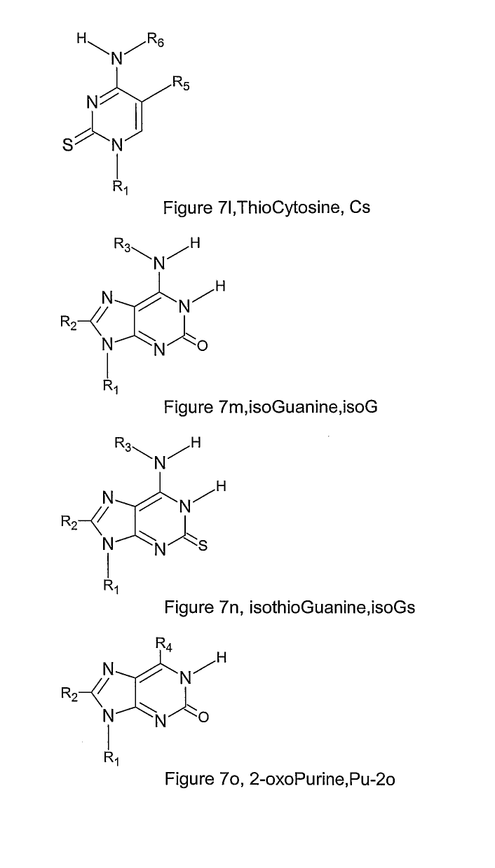

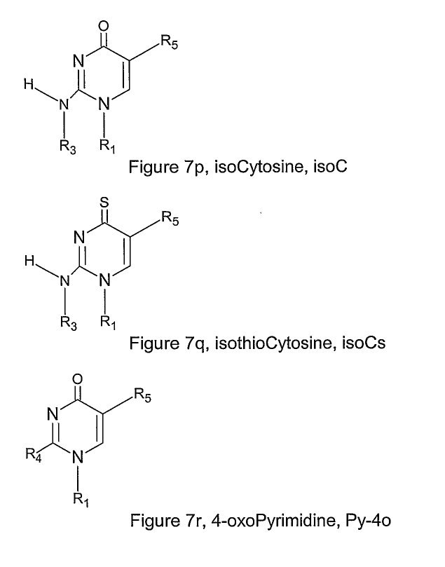

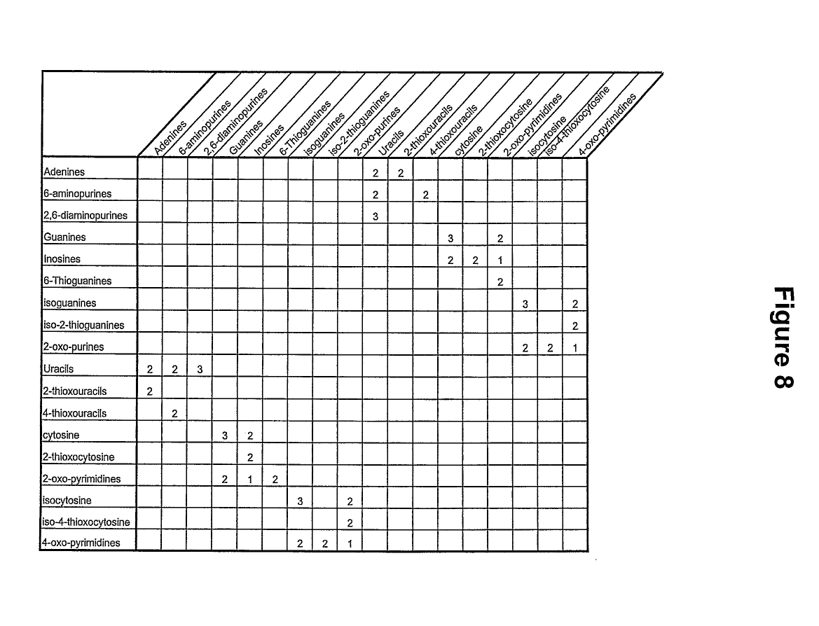

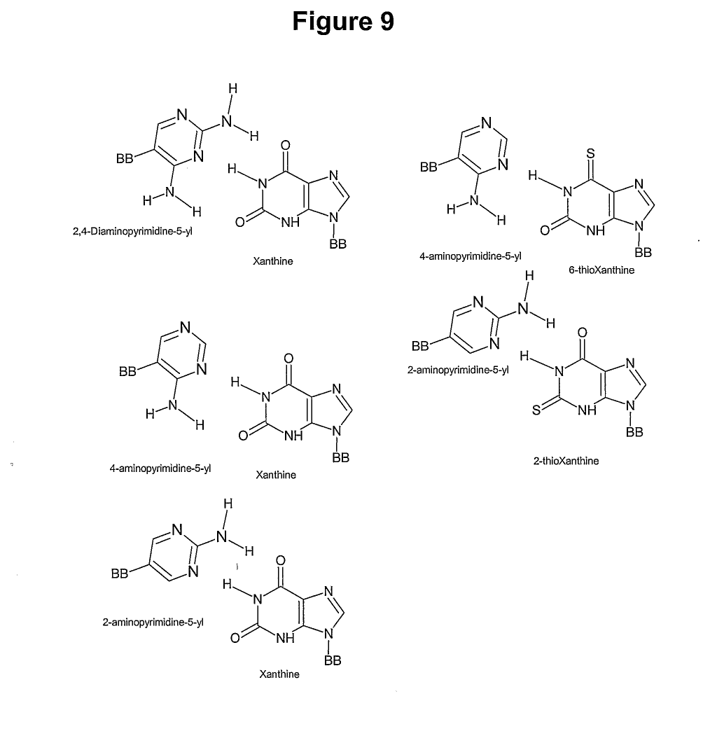

FIG. 8 shows interactions between each of the 18 bases shown in FIG. 7: 3 refers to three hydrogen bonds being present between the bases; 2 refers to two hydrogen bonds being present between the bases; 1 is the presence of one hydrogen bond; and X is a repulsion or no H bonding between the pairs. There are 3 three bond base pairs, 12 two bond base pairs, and 2 single bond base pairs. As may be seen from the figure and the text below, these pairing schemes may be used to expand the normal genetic code and thus may allow nucleic acid analog segments to specifically hybridize to more than one other nucleic acid analog segment within the instant recognition, adaptor, and detection units of the invention. See the accompanying International Application entitled “New Nucleic Acid Base Pairs” for further information on these pairing schemes.

FIG. 9 illustrates additional base pairs and bases compatible with detection systems used in the invention. See the accompanying International Application entitled “New Nucleic Acid Base Pairs” for further information on these pairing schemes.

### DEFINITIONS

Sample, as used herein, refers to any composition potentially containing a target.

Target, as used herein, refers to any substance present in a sample that is capable of detection.

A probe, as defined herein, comprises any substance that is capable of recognizing a target. In some embodiments of this invention, the probe is comprised in a larger molecular entity called a recognition unit herein, that could also comprise other functional elements, for instance, a polymer and/or linker segment, a detectable label, and/or an element that may be recognized by an adaptor unit or detectable label.

The terms recognize, recognition, or recognizing, etc., as used herein, mean an event in which one substance, such as a probe or recognition unit comprising a probe, directly or indirectly interacts with a target in any way such that the interaction with the target may be detected by a detection unit. In some non-limiting examples, a probe may react with a target, or directly bind to a target, or indirectly react with or bind to a target by directly binding to another substance that in turn directly binds to or reacts with a target.

The terms bind, binding, and similar terms, when applied to the instant targets and probes, mean an event in which one substance physically interacts with another in any way such that the interaction with the target may be detected. Specific, specific for, or specifically and similar terms when describing binding between two or more molecular entities mean that the binding is through specific interactions rather than through non-specific aggregation, for example. For instance, when nucleic acid segments are involved, specific binding may include formation of hydrogen bonds between the segments of Watson-Crick, wobble, and Hoogsteen base-pair geometries, such as to form double strands.

As used herein, a detection unit refers to a substance comprising at least one detectable label, and capable of binding directly to a recognition unit or indirectly to a recognition unit through an optional adaptor unit. In some embodiments of this invention, a detection unit also comprises at least one nucleic acid analog segment, hapten, antigen, binding agent, or other entity capable of specifically binding to another entity. In some embodiments, the detection unit may also comprise at least one polymer and/or at least one linker.

An adaptor unit, as used herein, means a substance that is capable of linking a recognition unit to a detection unit. In some embodiments of this invention, an adaptor unit comprises nucleic acid analog segments, haptens, antigens, binding agents, or other entities capable of specific binding to other entities. In some embodiments, the adaptor unit may also comprise at least one polymer and/or at least one linker.

A cross-linking agent or cross-linker herein is any substance that is capable of covalently attaching together molecules in a sample, such as a target and probe and/or attaching the sample to its container, such as a slide or plate or matrix or vessel.

The terms mask and masking, and similar terms, when used in the context of a target or other molecular entity, mean the reduction in the affinity of that target or molecular entity for a specific binding partner, such as a probe or adaptor unit. Masking may occur by a variety of means, such as steric hindrance, conformational changes, or substitution.

According to this invention, a detectable label is any molecule or functional group that allows for the detection of the presence of the target. A detectable label may also be comprised on a larger molecular entity that also comprises other functional elements such as linkers, polymers, and elements that recognize targets, probes, and/or adaptor units.

Amplify, amplification, and similar terms, mean an increase in the observed intensity of a signal from a detectable label.

A protein herein is used in the broadest possible sense, and includes any molecule comprising a sequence of amino acids, such as a short peptide, peptide hormone, or protein fragment, and larger molecules including antibodies, enzymes, glycoproteins, lipoproteins, etc.

A primary binding agent as used herein, refers to a substance that binds directly to a target in a sample.

A secondary binding agent, as used herein, refers to a substance which binds directly to a primary binding agent.

A tertiary binding agent, as used herein, refers to a substance which specifically binds a secondary binding agent.

Antibody, as used herein, means an immunoglobulin or a fragment thereof, and encompasses any polypeptide comprising an antigen-binding site regardless of the source, method of production, and other characteristics.

An antigen, as used herein, refers to any substance recognized by an antibody.

As used herein, the terms base and nucleobase refer to any purine-like or pyrimidine-like molecule that may be comprised in a nucleic acid segment or nucleic acid analog segment.

As used herein, a nucleic acid segment refers to a nucleobase sequence comprising any oligomer, polymer, or polymer segment, having a backbone formed solely from RNA or DNA nucleosides and comprising only the bases adenine (A), cytosine (C), guanine (G), thymine (T), and uracil (U), wherein an oligomer means a sequence of two or more nucleobases.

A non-natural base, as used herein, means any nucleobase other than: Adenine, A; Guanine, G; Urasil, U; Thymine, T; Cytosine, C.

A non-natural backbone unit includes any type of backbone unit to which a nucleobase may be attached that is not a ribose-phosphate (RNA) or a deoxyribose-phosphate (DNA) backbone unit.

As used herein, a nucleic acid analog segment means any oligomer, polymer, or polymer segment, comprising at least one monomer that comprises a non-natural base and/or a non-natural backbone unit.

As used herein, all numbers are approximate, and may be varied to account for errors in measurement and rounding of significant digits.

### DETAILED DESCRIPTION OF THE INVENTION

### Exemplary Methods

Some embodiments of the invention comprise automated methods of detecting at least one target in a sample, comprising:

a) obtaining a sample comprising at least one target;

b) contacting the sample with at least one probe specific for the at least one target;

c) contacting the sample with at least one detectable label; and

d) detecting the presence of the at least one target with the at least one detectable label;

wherein, following one or more of parts (a)-(c), the sample is incubated with at least one cross-linking agent; and wherein progress from one or more of parts (a)-(d) is automatically controlled.

Some embodiments of the instant invention include a method of detecting at least two targets in a sample, comprising:

a) obtaining a sample comprising at least one first target and at least one second target;

b) contacting the sample with at least one first probe specific for the at least one first target;

c) contacting the sample with at least one second probe specific for the at least one second target;

d) contacting the sample with at least one detectable label; and

e) detecting the presence of the at least one first target and at least one second target with the at least one detectable label;

wherein, following one or more of parts (a)-(d), the sample is incubated with at least one cross-linking agent.

Some embodiments of the invention comprise a method of detecting at least two targets in a sample, comprising:

a) obtaining a sample comprising at least one first target and at least one second target;

b) contacting the sample with at least one first probe specific for the at least one first target;

c) contacting the sample with at least one second probe specific for the at least one second target;

d) adding at least one adaptor unit specific for the at least one first probe and/or for the at least one second probe;

e) adding at least one detectable label specific for the at least one adaptor unit; and

f) detecting the presence of the at least one first target and at least one second target with the at least one detectable label;

wherein, following one or more of parts (a)-(d), the sample is incubated with at least one cross-linking agent.

In the methods above, the cross-linking agent may be added between parts (a) and (b), between parts (b) and (c), between parts (c) and (d), between parts (d) and (e), and/or between parts (e) and (f).

The adaptor unit allows the probe and detectable label to bind each other indirectly rather than directly, as each binds to the adaptor. In some embodiments, the adaptor functions to amplify the signal. It may comprise a secondary antibody, hapten, or engineered molecular entity, as described in more detail below.

Optionally, at some stage after adding the cross-link, the buffer conditions of the sample may be altered to those which would have been incompatible with the detection process prior to cross-linking. For example, the temperature may be changed, or there may be a change in pH, salt concentration, solvent, buffer substance, or addition of agents that would ordinarily chemically modify some element of the sample such as protease or nuclease enzymes.

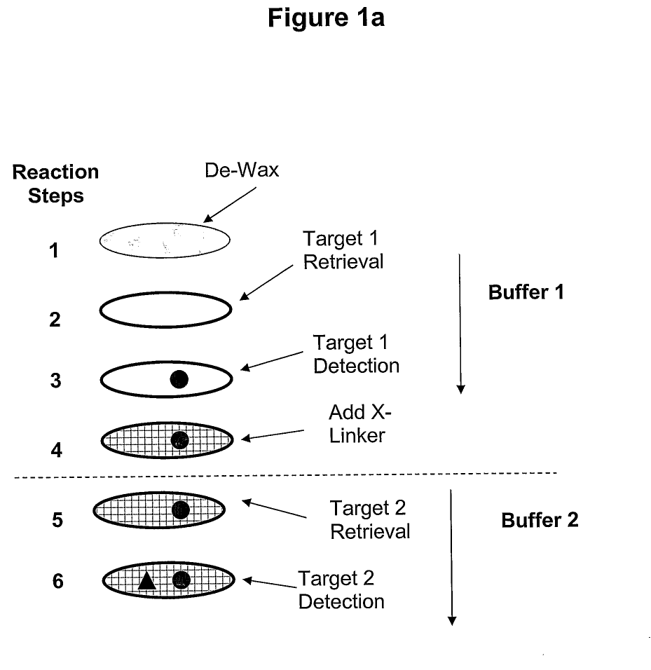

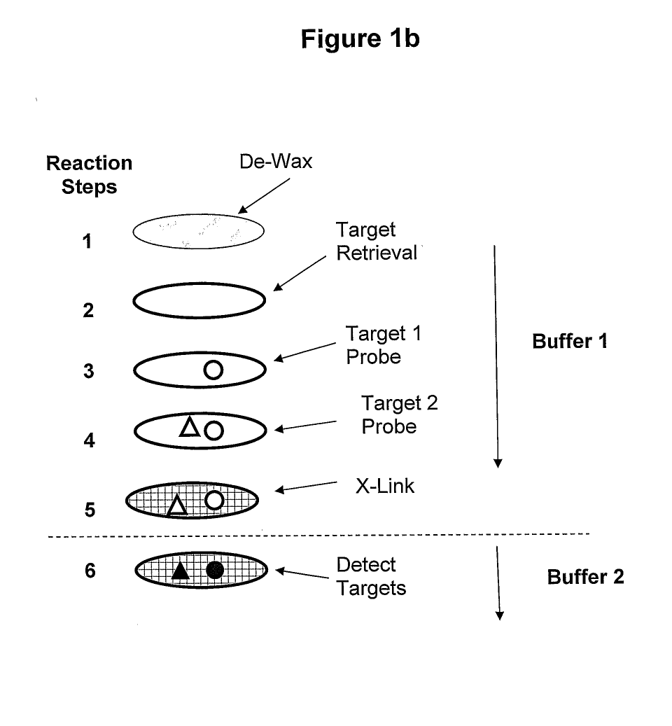

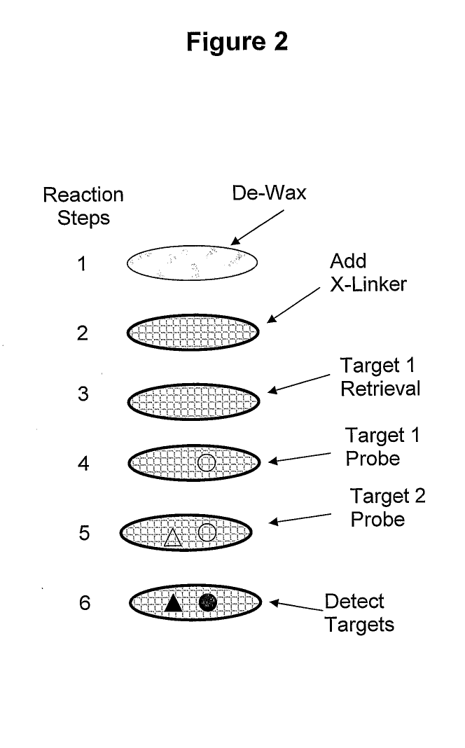

As described in the methods above, the cross-link may be employed at various points during an overall protocol. (See FIGS. 1-2 for examples.) For example, the cross-link may be employed as early as after de-waxing or de-paraffination of a sample, fixing the sample sufficiently to allow for the use of harsh conditions in later steps of the process. In such cases, cross-links between components of the sample or between the sample and the support may prevent a tissue or cytology sample from falling off the support or contaminating another sample. Cross-linking may also be employed after the first target is labeled, but prior to probing and labeling the second target, which in some embodiments allows one to choose later reagents, solvents, or washes more freely, with less consideration for the chemical nature of the previously detected, and now cross-linked target. (See FIG. 1a.) Hence, one may alter the wash, solvent, temperature, pH, or salt conditions to those that would normally be incompatible with the previously detected target. Similarly, one may detect one, two or more targets with a binding agent, probe, or optionally an adaptor and/or amplification reagent, and then cross-link the sample prior to adding detectable labels to visualize the samples. (See, e.g., FIG. 1b.) In such an example, one may use a detectable label that would normally be incompatible with the target. Cross-linkers may also be added more than once during the protocol, such as at any of the steps mentioned above.

More than one step of those methods may also be performed at the same time as one or more other steps, if that is compatible with the sample in question. For instance, the at least one first probe and at least one second probe could in some circumstances be added at the same time, or could be added with the at least one cross-linking agent and/or detectable label. In some embodiments, different detectable labels may be used for each target, while in others, the same detectable label may be used for both. In some embodiments, the detectable labels may also be specific for one or more probes. For example, at least one first probe may bind specifically to at least one first detectable label and an at least one second probe may bind specifically to at least one second detectable label. In such a system, for example, the first and second detectable labels may also be distinguishable from each other.

In some embodiments, it may be necessary to carry out target retrieval procedures as described below after obtaining the sample but prior to contacting it with the at least one first probe. A cross-linking agent may also be added before or after target retrieval, between parts (a) and (b) in the methods above. In some methods, one or more binding agents is added to detect the target prior to adding the probe. (See below.)

Either method may be automated and carried out in an automatic detection instrument, as described in more detail below. For instance, progression from one part of the method to a following part may be automatically controlled, such as via an automatic detection unit.

### Kits, Instruments, and Automation

The invention also provides a kit comprising one or more compositions according to the invention. The kit may optionally comprise one or more binding agents, and suitable reagents for, for instance: target retrieval, sample dilution, reagent dilution, blocking of non-specific binding, blocking of endogenous enzyme activity, or blocking of repetitive sequences. The kit may optionally also comprise at least one container, instructions for use, and reference targets or samples.

For example, a kit may comprise reagents for detecting one type of target, such as a protein, antigen, nucleic acid, etc., present in one or more containers, and reagents for detecting a second target, such as another protein, antigen, nucleic acid, etc., in one or more additional containers. It may contain appropriate wash buffers, for example, mixed with one or more detection reagents, or in separate containers. It may also contain the cross-linking agent in a separate container from the various detection reagents.

The present methods are compatible with automated staining protocols and equipment. In an automated method, the addition of the reagents, including the addition of the cross-linker, may be controlled by an automated machine with appropriate software telling the machine when and how much of the various reagents to dispense on the sample and providing instructions to move from one part of the method to the next. For instance, a machine can be programmed to dispense a probe, wash, then dispense a cross-linker, wash, etc. over the course of a detection protocol. Instruments capable of performing steps of staining, are useful for carrying out both single as well as multi-staining procedures, and, in particular, useful for detection of multiple targets that frequently requires balancing of the signals emanating from the different detectable labels.

### Cross-Linking Agents

The cross-linking agent creates covalent chemical bonds between components in the sample. Exemplary cross-linking agents include glutaric dialdehyde, vinyl sulfone, active esters, and adipic hydrazides. Other cross-linking agents include, for instance, 1-ethyl-3-[3-dimethylaminopropyl]carbodiimide HCl (EDC); N-hydroxysuccinimide (NHS); N-hydroxysulfosuccinimide (Sulfo-NHS); beta-[Tris(hydroxymethyl) phosphino]propionic acid (THPP); succinimidyl 3-[bromoacetamido]propionate) (SBAP); N-succinimidyl-S-acetylthioacetate) (SATA) and N-succinimidyl-S-acetylthioproprionate (SATP); N-succinimidyl (4-azidophenyl)-1,3′-dithiopropionate) (SADP); N[p-maleimidophenyl]isocyanate (PMPI); methyl N-succinimidyl adipate (MSA); 4-(r-N-maleimidophenyl) butyric acid hydrazine HCl (MPBH); N-k-maleimidoundecanoic acid (KMUA); 1,6-hexane-bis-vinylsulfone (HBVS); N-[g-maleimidobutyryloxy]succinimide ester (GMBS); N-e-maleimidocaproyloxy]succinimide ester (EMCS); N-e-maleimidocaproic acid (EMCA); ethylene glycol bis[succinimidylsuccinate] (EGS); dithio-bis-maleimidoethane (DTME); dimethyl 3,3′-dithiobispropionimidate-2 HCL (DTBP); disuccinyl tartarate (DST); disuccinimidly glutarate (DSG); 1,4-Di-[3′-(2′.pyridyldithio)-propionamido]butane (DPDPB); dimethyl suberimidate-2 HCL (DMS); and dimethyl adipimidate-2 HCl (DMA).

Suitable cross-linkers are also described in, for example, Wong, S. S., Chemistry of Protein Conjugation and Cross-Linking, CRC Press: Boca Raton, Fla., USA (1993), and in G T Hermanson et al., Immobilization Affinity Ligand Techniques, Academic Press: San Diego, Calif., USA (1992), and in commercial catalogues available from Pierce Chemical Co., Rockford Ill., USA, and other suppliers.

The above and other suitable cross-linking agents may be chosen depending on the functional groups present on the target and/or probe for example. For instance, some cross-linking agents primarily react with OH or SH groups, while others primarily react with carbonyls, and still others primarily react with amine groups.

In some embodiments, a UV curable polymer, or radical polymerizable monomers or homopolymers may be used as cross-linking agents. Some embodiments use a cross-linker chosen from at least one of formaldehyde, paraformaldehyde, a di or poly-aldehyde such as glutaric dialdehyde, and divinyl sulfone.

In some embodiments, the cross linker is stable upon storage at room temperature. A fast-reacting cross-linking agent may also be employed, such as one that is chemically reactive enough to form appropriate cross-links within minutes of application. In some embodiments, a reversible cross-link may be generated. Cross-linkers may have different lengths. For example, they may comprise small molecules forming covalent bonds between closely-spaced atoms, or they may comprise reactive entities separated by molecular linkers, to allow cross-links between atoms spaced at greater distances.

The cross-linking agent may also be mixed with or may comprise tissue fixation reagents such as 4% formaldehyde, glutaric dialdehyde, Bouin's Fluid, picric acid, acetic acid, diazolidinyl urea, 2-bromo-2-nitropropane-1,3-diol, zinc salts such as zinc sulfate, and mercuric salts such as mercuric chloride. Certain fixatives both covalently cross-link components of the sample and also denature and coagulate proteins. In the present invention, however, it is not always desirable to denature and coagulate proteins; hence in some embodiments, a cross-linker is chosen that covalently cross-links components of the sample but does not significantly denature and/or coagulate proteins under the buffer conditions chosen. For instance, in some samples, protein denaturation and coagulation makes retrieval of a second or third target more difficult, as a large network of cross-links is created in the sample rendering those targets less accessible to further probes and other detection reagents. In such embodiments, fixatives such as formaldehyde, paraformaldehyde, glutaric dialdehyde, formalin, and alcohols, and optionally Bouin's Fluid, picric acid, acetic acid, diazolidinyl urea, 2-bromo-2-nitropropane-1,3-diol, zinc salts such as zinc sulfate, and mercuric salts such as mercuric chloride are specifically excluded from the selected cross-linkers. On the other hand, in some embodiments, the cross-linking agent may be mixed with solvents that cause coagulation or irreversable fixation of a sample, including water, methanol, ethanol, other alcohols, dimethylsulfoxide, acetic acid, and acetone, if that does not interfere with later detection method steps.

In some embodiments, probes, detectable labels, other detection reagents, and the molecular entities in which they are comprised may be engineered to add a functional group that is reactive with the cross-linker. Such embodiments may allow for specific control of the cross-linking process by targeting the cross-linking agent to the engineered sites.

In yet other embodiments, it is advantageous to use a near-zero-length cross-linker to form covalent bonds between components in the sample without forming networks of cross-links that render later targets difficult to access. Zero-length or near-zero-length cross-links also help to prevent diffusion of components in the sample as the detection method progresses, hence allowing for sharper visualization.

### Samples

Many types of samples are compatible with the instant invention. Samples may comprise solid and liquid solutions, for example, containing targets in a buffer. Samples may also be derived from living matter taken from any living organism, e.g., an animal, such as mammals (e.g. humans), plants, fungi, archaea, or bacteria. Thus, samples may comprise eukaryotic cells, archaeal cells, or prokaryotic cells. Samples may comprise a cell sample, such as a cell smear or colony, or a tissue specimen derived from a living organism, such as a tissue sample from an organ. They may also comprise a biological fluid, such as an animal-derived fluid, e.g. mammalian plasma, serum, lymph, whole blood, spinal, amniotic, or other fluid. Samples may also comprise other naturally-obtained samples such as soil or water samples, and synthetically derived samples such as chemical or industrial products or solutions, food products, and buffers.

### Targets

The instant invention can be applied to a variety of targets. In some embodiments, the target comprises a protein or nucleic acid. Targets may also include cell or viral particles, or portions thereof, e.g., a nucleic acid segment or a protein. The cell or viral particle may be a free viral particle, i.e., not associated with any other molecule, or it may be associated with any sample described above. In some embodiments, the target may be an antigen or an antibody. In some embodiments, the target comprises a lipid; a glyco-lipid; a sugar; a polysaccharide; a starch; a salt; an ion; or one of a variety of other organic and inorganic substances; any of which may be free in solution or bound to another substance.

In some embodiments of the invention, the first and second targets according to the invention do not comprise nucleic acids. For instance, the first target and second target may comprise only proteins in certain embodiments, or only proteins and larger structures such as organelles in others. In some such embodiments, no targets are nucleic acids. In other embodiments, however, the first and second targets both comprise nucleic acids. Such embodiments include those in which no proteins or other structures are targeted, for example, different DNAs or RNAs may targeted with different probes and/or detectable labels. In yet other embodiments, the targets comprise, for example, one or more nucleic acids as well as one or more proteins or other structures. In some embodiments, the first target is not a nucleic acid, while the second target is a nucleic acid. In other embodiments of the invention, the first target is a nucleic acid while the second target is not a nucleic acid. For example, one target may be a protein while the other is a nucleic acid.

The target may be expressed on the surface of the sample, e.g., such as on a membrane or interface. Alternatively, the target may be contained in the interior of the sample. In the case of a cell sample, for instance, an interior target may comprise a target located within the cell membrane, periplasmic space, cytoplasm, or nucleus, or within an intracellular compartment or organelle.

### Detection Systems Compatible with the Invention

The instant invention is compatible with many known detection formats and their associated samples. For example, the invention may be used in connection with immunoassays, protein detection assays, or nucleic acid hybridization assays such as: immunohistochemistry (IHC), immunocytochemistry (ICC), in situ hybridization (ISH), flow cytometry, enzyme immuno-assays (EIA), enzyme linked immuno-assays (ELISA), blotting methods (e.g. Western, Southern, and Northern), labeling inside electrophoresis systems or on surfaces or arrays, and precipitation, among others. All of those detection assays are useful in research as well as in the detection and diagnosis of a variety of diseases and conditions, for example.

### IHC and ISH Detection Systems

For example, IHC specifically provides a method of detecting targets in a sample or tissue specimen in situ (see Mokry 1996, ACTA MEDICA 39:129). The overall cellular integrity of the sample is maintained in IHC, thus allowing detection of both the presence and location of the targets of interest. Typically a sample is fixed with formalin, embedded in paraffin and cut into sections for staining and subsequent inspection by light microscopy. Current methods of IHC use either direct labeling or secondary antibody-based or hapten-based labeling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

IHC, ISH and cytological techniques may be performed in a matrix of tissue, cell and proteins which may be partly cross-linked and very inhomogeneous in nature. Diffusion rates increase with increasing concentrations and increasing temperature, but decrease with molecular weight and molecular size. Therefore, the physical size of the components is of great importance. For instance, large molecules can be excluded from diffusing into parts of the sample whereas small sized components more easily may diffuse in and out of the different compartments of the sample. In some embodiments, the units of the invention may be designed to be of small size and, for example, smaller than an antibody or biotin-streptavidine complex, in order to improve target recognition and detection.

### Preparation of Cell and Tissue Samples

Tissue or cell samples according to the invention may be prepared by a variety of methods known to those of ordinary skill in the art, depending on the type of sample and the assay format. For instance, tissue or cell samples may be fresh or preserved, and may be, for example, in liquid solution, flash-frozen or lyophilized, smeared or dried, embedded, or fixed on slides or other supports. In some embodiments, samples may be prepared and stained using a free-floating technique. In this method a tissue section is brought into contact with different reagents and wash buffers in suspension or freely floating in appropriate containers, for example micro centrifuge tubes, before being mounted on slides for further treatment and examination.

In some embodiments, a tissue section may be mounted on a slide or other support after an incubation with immuno-specific reagents. The remains of the staining process are then conducted after mounting. For example, for microscopic inspection in IHC and ISH, samples may be comprised in a tissue section mounted on a suitable solid support. For the production of photomicrographs, sections comprising samples may be mounted on a glass slide or other planar support, to highlight by selective staining certain morphological indicators of disease states or detection of detectable targets.

In some IHC embodiments, a sample may be taken from an individual, fixed and exposed to, for example, antibodies which specifically bind to the detectable target of interest. Sample processing steps may include, for example, antigen retrieval, exposure to a primary antibody, washing, exposure to a secondary antibody (optionally coupled to a suitable detectable label), washing, and exposure to a tertiary antibody linked to a detectable label. Washing steps may be performed with any suitable buffer or solvent, e.g., phosphate-buffered saline, TRIS-buffered saline, distilled water. The wash buffer may optionally contain a detergent, e.g., TWEEN® 20 or NP-40.

IHC samples may include, for instance: (a) preparations comprising un-fixed fresh tissues and/or cells or solution samples (b) fixed and embedded tissue specimens, such as archived material; and (c) frozen tissues or cells. In some embodiments, an IHC staining procedure may comprise steps such as: cutting and trimming tissue, fixation, dehydration, paraffin infiltration, cutting in thin sections, mounting onto glass slides, baking, deparaffination, rehydration, antigen retrieval, blocking steps, applying primary antibody, washing, applying secondary antibody—enzyme conjugate, washing, applying a tertiary antibody conjugated to a polymer and linked with an enzyme, applying a chromogen substrate, washing, counter staining, applying a cover slip and microscopic examination.

ISH samples, for instance, may be taken from an individual and fixed before being exposed to a nucleic acid or nucleic acid analog probe on a recognition unit. In some embodiments, the nucleic acid in the sample may first be denatured to expose the target binding sites. Various counter-stains or paints may further be used in order to locate nucleic acid molecules or chromosomes within an ISH sample.

### Methods of Fixing Cell and Tissue Samples

In some embodiments of this invention, tissue or cell samples may be fixed or embedded before the detection process begins. Fixatives may be needed, for example, to preserve cells and tissues in a reproducible and life-like manner. Fixatives may also stabilize cells and tissues, thereby protecting them from the rigors of processing and staining techniques. For example, samples comprising tissue blocks, sections, or smears may be immersed in a fixative fluid, or in the case of smears, dried.

Many methods of fixing and embedding tissue specimens are known, for example, alcohol fixation and formalin-fixation and subsequent paraffin embedding (FFPE). Any suitable fixing agent may be used. Examples include ethanol, acetic acid, picric acid, 2-propanol, 3,3′-diaminobenzidine tetrahydrochloride dihydrate, acetoin (mixture of monomer) and dimer, acrolein, crotonaldehyde (cis+trans), formaldehyde, glutaraldehyde, glyoxal, potassium dichromate, potassium permanganate, osmium tetroxide, paraformaldehyde, mercuric chloride, tolylene-2,4-diisocyanate, trichloroacetic acid, tungstic acid. Other examples include formalin (aqueous formaldehyde) and neutral buffered formalin, glutaraldehyde, carbodiimide, imidates, benzoequinone, osmic acid and osmium tetraoxide. Fresh biopsy specimens, cytological preparations (including touch preparations and blood smears), frozen sections, and tissues for IHC analysis may be fixed in organic solvents, including ethanol, acetic acid, methanol and/or acetone.

### Detectable Labels

A detectable label according to the invention may include any molecule which may be detected directly or indirectly so as to reveal the presence of a target in the sample. In some embodiments of the invention, a direct detectable label is used. Direct detectable labels may be detected per se without the need for additional molecules. Examples include fluorescent dyes, radioactive substances, and metal particles. In other embodiments of the invention, indirect detectable labels are used, which require the employment of one or more additional molecules. Examples include enzymes that affect a color change in a suitable substrate, as well as any molecule that may be specifically recognized by another substance carrying a label or react with a substance carrying a label. Other examples of indirect detectable labels thus include antibodies, antigens, nucleic acids and nucleic acid analogs, ligands, substrates, and haptens.

Examples of detectable labels which may be used in the invention include fluorophores, chromophores, electrochemiluminescent labels, bioluminescent labels, polymers, polymer particles, bead or other solid surfaces, gold or other metal particles or heavy atoms, spin labels, radioisotopes, enzyme substrates, haptens, antigens, Quantum Dots, aminohexyl, pyrene, nucleic acids or nucleic acid analogs, or proteins, such as receptors, peptide ligands or substrates, enzymes, and antibodies (including antibody fragments).

Some detectable labels according to this invention comprise “color labels,” in which the target is detected by the presence of a color, or a change in color in the sample. Examples of “color labels” are chromophores, fluorophores, chemiluminescent compounds, electrochemiluminescent labels, bioluminescent labels, and enzymes that catalyze a color change in a substrate. In some embodiments, more than one type of color may be used, for instance, by attaching distinguishable color labels to a single detection unit or by using more than one detection unit, each carrying a different and distinguishable color label.

“Fluorophores” as described herein are molecules that emit detectable electro-magnetic radiation upon excitation with electro-magnetic radiation at one or more wavelengths. A large variety of fluorophores are known in the art and are developed by chemists for use as detectable molecular labels and can be conjugated to the linkers of the present invention. Examples include fluorescein or its derivatives, such as fluorescein-5-isothiocyanate (FITC), 5-(and 6)-carboxyfluorescein, 5- or 6-carboxyfluorescein, 6-(fluorescein)-5-(and 6)-carboxamido hexanoic acid, fluorescein isothiocyanate, rhodamine or its derivatives such as tetramethylrhodamine and tetramethylrhodamine-5-(and-6)-isothiocyanate (TRITC). Other example fluorophores that could be conjugated to the instant linkers include: coumarin dyes such as (diethyl-amino)coumarin or 7-amino-4-methylcoumarin-3-acetic acid, succinimidyl ester (AMCA); sulforhodamine 101 sulfonyl chloride (TexasRed™ or TexasRed™ sulfonyl chloride; 5-(and-6)-carboxyrhodamine 101, succinimidyl ester, also known as 5-(and-6)-carboxy-X-rhodamine, succinimidyl ester (CXR); lissamine or lissamine derivatives such as lissamine rhodamine B sulfonyl Chloride (LisR); 5-(and-6)-carboxyfluorescein, succinimidyl ester (CFI); fluorescein-5-isothiocyanate (FITC); 7-diethylaminocoumarin-3-carboxylic acid, succinimidyl ester (DECCA); 5-(and-6)-carboxytetramethylrhodamine, succinimidyl ester (CTMR); 7-hydroxycoumarin-3-carboxylic acid, succinimidyl ester (HCCA); 6->fluorescein-5-(and-6)-carboxamidolhexanoic acid (FCHA); N-(4,4-difluoro-5,7-dimethyl-4-bora-3a,4a-diaza-3-indacenepropionic acid, succinimidyl ester; also known as 5,7-dimethylBODIPY™ propionic acid, succinimidyl ester (DMBP); “activated fluorescein derivative” (FAP), available from Molecular Probes, Inc.; eosin-5-isothiocyanate (EITC); erythrosin-5-isothiocyanate (ErITC); and Cascade™ Blue acetylazide (CBAA) (the O-acetylazide derivative of 1-hydroxy-3,6,8-pyrenetrisulfonic acid). Yet other potential fluorophores useful in this invention include fluorescent proteins such as green fluorescent protein and its analogs or derivatives, fluorescent amino acids such as tyrosine and tryptophan and their analogs, fluorescent nucleosides, and other fluorescent molecules such as Cy2, Cy3, Cy 3.5, Cy5, Cy5.5, Cy 7, IR dyes, Dyomics dyes, phycoerythrine, Oregon green 488, pacific blue, rhodamine green, and Alexa dyes. Yet other examples of fluorescent labels which may be used in the invention include and conjugates of R-phycoerythrin or allophycoerythrin, inorganic fluorescent labels such as particles based on semiconductor material like coated CdSe nanocrystallites.

A number of the fluorophores above, as well as others, are available commercially, from companies such as Molecular Probes, Inc. (Eugene, Oreg.), Pierce Chemical Co. (Rockford, Ill.), or Sigma-Aldrich Co. (St. Louis, Mo.).

Examples of polymer particles labels which may be used in the invention include micro particles, beads, or latex particles of polystyrene, PMMA or silica, which can be embedded with fluorescent dyes, or polymer micelles or capsules which contain dyes, enzymes or substrates.

Examples of metal particles which may be used in the invention include gold particles and coated gold particles, which can be converted by silver stains.

Examples of haptens that may be conjugated in some embodiments are fluorophores, myc, nitrotyrosine, biotin, avidin, strepavidin, 2,4-dinitrophenyl, digoxigenin, bromodeoxy uridine, sulfonate, acetylaminoflurene, mercury trintrophonol, and estradiol.

Examples of enzymes which may be used in the invention comprise horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase and glucose oxidase (GO).

Examples of commonly used substrates for horse radish peroxidase (HRP) include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), α-naphtol pyronin (α-NP), o-dianisidine (OD), 5-bromo-4-chloro-3-indolylphosphate (BCIP), Nitro blue tetrazolium (NBT), 2-(p-iodophenyl)-3-p-nitrophenyl-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B1-phosphate/fast red TR (NABP/FR), Naphthol-AS-MX-phosphate/fast red TR (NAMP/FR), Naphthol-AS-B1-phosphate/fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b(beta)-d (delta)-galactopyranoside (BCIG).

Examples of luminescent labels which may be used in the invention include luminol, isoluminol, acridinium esters, 1,2-dioxetanes and pyridopyridazines. Examples of electrochemiluminescent labels include ruthenium derivatives.

Examples of radioactive labels which may be used in the invention include radioactive isotopes of iodide, cobalt, selenium, hydrogen, carbon, sulfur and phosphorous.

In some embodiments a signal amplification may allow for 1 up to 500 detectable label molecules per probe. For example, a primary antibody probe may be contacted with a secondary antibody conjugated to a detectable label. As another example, in some embodiments, the detectable label is an enzyme, which may be conjugated to a polymer, such that the number of enzyme molecules conjugated to each polymer molecule is, for instance, 1 to 200, 2 to 50, or 2 to 25. In some embodiments, the detectable label is a gold particle, a radioactive isotope, or a color label, e.g. a low molecular weight fluorochrome, and the number of detectable labels conjugated to each polymer molecule is, for instance, 1 to 500, or for instance, 2 to 200. In some embodiments, the detectable label is a protein fluorochrome and the number of detectable labels conjugated to each polymer molecule is 1-50, 2-20. In some embodiments, the number of detectable label molecules conjugated to each polymer is 1-200, 2-50, 2-25, or is 10-20, 5-10, or 1-5.

The detectable label can be detected by numerous methods, including, for example, reflectance, transmittance, light scatter, optical rotation, and fluorescence or combinations hereof in the case of optical labels or by film, scintillation counting, or phosphorimaging in the case of radioactive labels. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.). In some embodiments, more than one detectable label is employed.

When more than one color label is used, the different colors may have different, distinguishable colors. In some embodiments both colors can be detected simultaneously, such as by fusion or juxtaposition of the signals, signal enhancement or quenching, or detection of multiple colors in the sample. The exact choice of detectable label or combinations of detectable labels may be based on personal preferences in combinations with restrictions of the sample type, sample preparation method, detection method and equipment, and optional contrasting labels used in the sample.

### Probes

The instant invention is further compatible with a variety of types of probes. In some embodiments, the recognition may be direct, such as through non-covalent or covalent binding between the probe and the target. In other embodiments, the recognition may be indirect, such as through another binding agent. In yet other embodiments, the probe may react directly or indirectly with a target to render the target detectable. In some embodiments, the probe comprises a nucleic acid or protein or a ligand such as an enzyme substrate, antigen, hapten, or cofactor.

In certain embodiments, the invention comprises at least one additional binding agent, such as a primary binding agent which directly binds to a target in a sample. In embodiments comprising one or more binding agents, the probe may directly bind to one or more of the binding agents rather than to the target itself. In other embodiments, the primary binding agent is an antibody, i.e., a primary antibody. In other embodiments the primary binding agent is a nucleic acid. In yet other embodiments the primary binding agent is a receptor, hapten, substrate, or a ligand.

Other embodiments of the invention further comprise a secondary binding agent. The secondary binding agent may be any molecule that binds the primary binding agent. For example, in some embodiments the primary binding agent is a primary antibody. In those embodiments, the secondary binding agent may comprise e.g. a secondary antibody, a Fc receptor or C1q, a protein from the classical pathway of the complement cascade. Depending on the primary binding agent, the secondary binding agent may be e.g an anti-hapten antibody, an MHC molecule, such as an MHC class I and MHC class II and non conventional MHC, a molecule having a specific binding partner, such as molecules involved in cellular signaling pathways or molecules having leucine zipper domains, e.g., fos/jun, myc, GCN4, molecules having SH1 or SH2 domains, such as Src or Grb-2. A secondary binding agent may also be comprised of a chimeric or a fusion protein, i.e., a protein engineered to combine the features of two or more specific binding partners. For instance, a leucine zipper could be engineered into an Fc region of an antibody or an SH2 domain could be engineered to be expressed in an Fc region of an antibody. The secondary binding agent may also comprise a hapten, such as fluorophores, myc, nitrotyrosine, biotin, avidin, strepavidin, 2,4-dinitrophenyl, digoxigenin, bromodeoxy uridine, sulfonate, acetylaminoflurene, mercury trintrophonol, and estradiol. The secondary binding agent may comprise a nucleic acid molecule that binds to a complementary nucleic acid molecule of the primary binding agent.

Yet other embodiments of the invention may comprise a tertiary binding agent that binds the secondary binding agent. The tertiary binding agent may comprise, for example, a tertiary antibody or a nucleic acid molecule or any of the specific binding partners described above for the secondary binding agent, so long as it binds the secondary binding agent. Certain embodiments of the invention may further comprise additional forth, fifth, or even higher order, binding agents similar to the binding agents described above.

### Binding Agents

In certain embodiments, where a binding agent is employed, the primary binding agent is an antibody, i.e., a primary antibody. In other embodiments the primary binding agent is a nucleic acid segment or nucleic acid analog segment. In yet other embodiments the primary binding agent is a receptor, hapten, substrate, or a ligand.

Other embodiments of the invention further comprise a secondary binding agent. The secondary binding agent may be any molecule that binds the primary binding agent. For example, in some embodiments the primary binding agent is a primary antibody. In those embodiments, the secondary binding agent may comprise e.g. a secondary antibody, a Fc receptor or C1q, a protein from the classical pathway of the complement cascade. Depending on the primary binding agent, the secondary binding agent may be e.g an anti-hapten antibody, an MHC molecule, such as an MHC class I and MHC class II and non conventional MHC, a molecule having a specific binding partner, such as molecules involved in cellular signaling pathways or molecules having leucine zipper domains, e.g., fos/jun, myc, GCN4, molecules having SH1 or SH2 domains, such as Src or Grb-2. A secondary binding agent may also be comprised of a chimeric or a fusion protein, i.e., a protein engineered to combine the features of two or more specific binding partners. For instance, a leucine zipper could be engineered into an Fc region of an antibody or an SH2 domain could be engineered to be expressed in an Fc region of an antibody. The secondary binding agent may also comprise a hapten, such as fluorophores, myc, nitrotyrosine, biotin, avidin, strepavidin, 2,4-dinitrophenyl, digoxigenin, bromodeoxy uridine, sulfonate, acetylaminoflurene, mercury trintrophonol, and estradiol. The secondary binding agent may comprise a nucleic acid molecule that specifically hybridizes to a complementary nucleic acid molecule of the primary binding agent.

Yet other embodiments of the invention may comprise a tertiary binding agent that binds the secondary binding agent. The tertiary binding agent may comprise, for example, a tertiary antibody or a nucleic acid molecule or any of the specific binding partners described above for the secondary binding agent, so long as it specifically binds the secondary binding agent. Certain embodiments of the invention may further comprise additional forth, fifth, or even higher order, binding agents similar to the binding agents described above.

### Antibodies

Antibodies may be used as detectable labels, targets, binding agents, or probes, for example, depending on the specifics of the detection method. Some embodiments may comprise, for example, polyclonal, monoclonal, monospecific, polyspecific, humanized, single-chain, chimeric, synthetic, recombinant, hybrid, mutated, and CDR-grafted antibodies. Various techniques for producing antibodies and preparing recombinant antibody molecules are known in the art and have been described, see, e.g., Kohler and Milstein, (1975) Nature 256:495; Harlow and Lane, Antibodies: a Laboratory Manual, (1988) (Cold Spring Harbor Press, Cold Spring Harbor, N.Y.). Antibodies used in the invention may be derived from any mammal species, e.g., rat, mouse, goat, guinea pig, donkey, rabbit, horse, lama, camel, or any avian species e.g., chicken, duck. The origin of the antibody is defined by the genomic sequence irrespective of the method of production.

The antibody may be of any isotype, e.g., IgG, IgM, IgA, IgD, IgE or any subclass, e.g., IgG1, IgG2, IgG3, IgG4. The skilled artisan will appreciate that antibodies produced recombinantly, or by other means, for use in the invention include any antibody fragment which can still bind antigen, e.g. an Fab, an F(ab)₂, Fv, scFv. In certain embodiments, the antibody, including an antibody fragment, may be recombinantly engineered to include a hapten, e.g, a peptide. In certain embodiments the hapten may be a myc tag (se FIG. 1N). Inclusion of a hapten in an antibody or antibody fragment facilitates subsequent binding of a binding agent, probe, or label

Certain embodiments employ a primary antibody contains an antigen binding region which can specifically bind to an antigen target in a sample. Thus, a primary antibody may act as either a primary binding agent or a probe in such embodiments, by directly recognizing the antigen target.

Some embodiments further employ a secondary antibody containing an antigen binding region which specifically binds to the primary antibody, e.g., the constant region of the primary antibody. In certain embodiments, the secondary antibody is conjugated to a polymer. In some embodiments, the polymer is conjugated with 2-20 secondary antibodies. In other embodiments, the polymer is conjugated with 2-10 secondary antibodies. In other embodiments, the polymer is conjugated with 1-5 tertiary antibodies, such as 1, 2, 3, 4, or 5. In some such embodiments, the secondary antibody acts as a secondary binding agent, while in other such embodiments, the secondary antibody acts as a probe, recognizing the target antigen indirectly through a primary antibody.

Some embodiments also employ a tertiary antibody containing an antigen binding region which specifically binds to the secondary antibody, e.g., a constant region of the secondary antibody, or a hapten linked to the secondary antibody or a polymer conjugated to the secondary antibody. In certain embodiments, the tertiary antibody is conjugated to a polymer. In some embodiments, the polymer is conjugated with 1-20 tertiary antibodies. In other embodiments, the polymer is conjugated with 1-5 tertiary antibodies, such as 1, 2, 3, 4, or 5. In some such embodiments, the tertiary antibody acts as a tertiary binding agent, while in other such embodiments, the tertiary antibody acts as a probe, recognizing the target antigen indirectly through a primary and a secondary antibody.

### Quantitation Methods

In some embodiments, the approximate amount of a target in a sample is also determined. For instance, a control target within the sample may be assayed as well as an experimental target. In the case of a nucleic acid target, for example, a chromosomal paint or counter-stain may be used. For instance, if the target is a locus on a larger piece of nucleic acid such as a plasmid or chromosome, the intensity of a contrasting label for the plasmid or chromosome or a neutral locus thereon may be compared to the intensity of the target locus. The intensity of the label from the sample may also be compared to that of a known standard or control sample. Estimating the amount of a detectable target in a sample is helpful, for instance, in a variety of diagnostic tests, and the estimate may be used to plan a course of treatment for a suspected disease or condition. Several commercial densitometry software programs and related instruments are available to quantitate the intensity of a stained target in a sample, such as those available from Fuji Film, Applied Biosystems, and Molecular Dynamics.

### Increasing the Reactivity and Specificity of Detectable Targets

In some embodiments, it may be helpful to treat samples before contacting them with probes in order to increase the reactivity or accessibility of a target and to reduce non-specific interactions. Further, more than one treatment procedure may be used, for example, when two different types of target molecules are to be detected. For instance, a first target may be detected with a first probe, and cross-linked. Then the sample may be treated to retrieve a second target under conditions in which the first cross-linking remains stable. Such treatments may involve, in some cases, even drastic changes in buffer conditions, pH, pressure, and temperature.

If the target is an antigen, for example, a sample may be treated with a process called “antigen retrieval” (and which is also known in the art as target retrieval, epitope retrieval, target unmasking, or antigen unmasking). See, e.g., Shi et al., J Histochem Cytochem, 45(3): 327 (1997). Antigen retrieval encompasses a variety of methods including enzymatic digestion with proteolytic enzymes, such as e.g. proteinase, pronase, pepsin, papain, trypsin or neuraminidase. Some embodiments may use heat, e.g. “heat-induced epitope retrieval” or HIER. Heating may involve a microwave irradiation, or a water bath, a steamer, a regular oven, an autoclave, or a pressure cooker in an appropriately pH stabilized buffer, usually containing EDTA, EGTA, Tris-HCl, citrate, urea, glycin-HCl or boric acid. One may add detergents to the HIER buffer to increase the epitope retrieval, or to the dilution media and/or rinsing buffers to lower non-specific binding. In some embodiments, combinations of different antigen retrieval methods may be used.

The antigen retrieval buffer may be aqueous, but may also contain other solvents, including solvents with a boiling point above that of water such as e.g glycerol. This allows for treatment of the tissue at more than 100° C. at normal pressure.

Additionally, in some embodiments, the signal-to-noise ratio may be increased by different physical methods, including application of vacuum, or ultrasound, or freezing and thawing tissue samples before or during incubation of the reagents.

In some embodiments, treatments may be performed to reduce non-specific binding. For example, carrier proteins, carrier nucleic acid molecules, salts, or detergents may reduce or prevent non-specific binding. Non-specific binding sites may be blocked in some embodiments with inert proteins like, HSA, BSA, ovalbumin, with fetal calf serum or other sera, or with detergents like TWEEN®20, TRITON® X-100, Saponin, BRIJ®, or PLURONICS®. Alternatively, non-specific binding sites may be blocked with unlabeled competitors for the recognition event between the target and the probe. For example, in the case of a nucleic acid interaction, non-specific binding may be reduced by adding unlabeled competitor nucleic acids or nucleic acid analogs such as digested, total human DNA or salmon sperm DNA, or unlabeled versions of the binding agent. In addition, repetitive sequences may be blocked, for example, using nucleic acids or nucleic acid analogs that specifically recognize those sequences, or sequences derived from a total DNA preparation. Salt, buffer, and temperature conditions may also be modified so as to reduce non-specific binding.

Cross reactivity of different components of the detection methods may be avoided, for example, by using antibodies derived from different species. Furthermore, combinations of e.g. secondary antibodies against primary antibodies and haptens may also be used to avoid unwanted cross reactivity. Alternatively, unwanted cross-reactivity or non-specific binding may be reduced or eliminated by designing sterically hindered probes, adaptor units, and/or detectable labels. In addition, one may remove endogenous biotin binding sites or endogenous enzyme activity (for example phosphatase, catalase or peroxidase). Endogenous biotin and peroxidase activity may be removed by treatment with peroxides, while endogenous phosphatase activity may be removed by treatment with levamisole. Heating may destroy endogenous phosphatase and esterase activity.

### Systems of Engineered Molecular Entities Compatible with the Invention

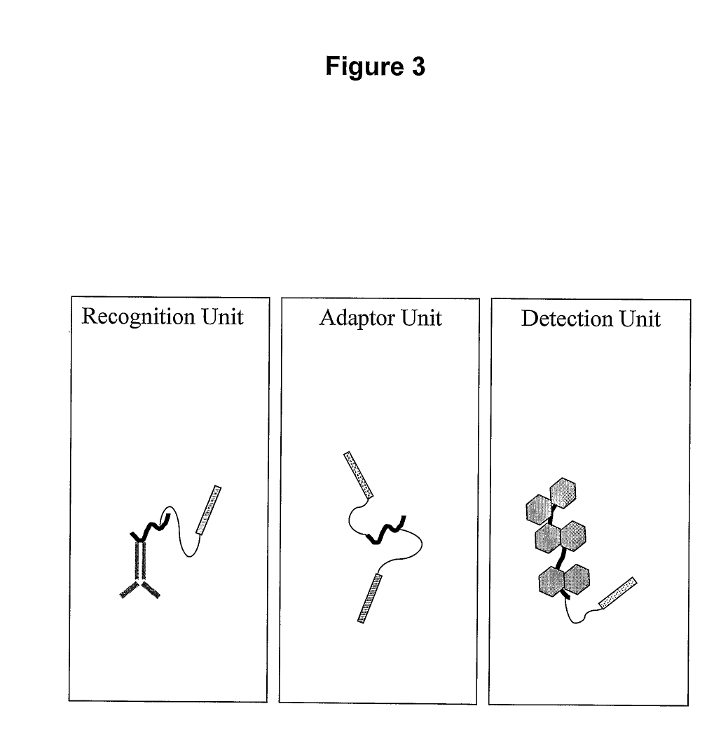

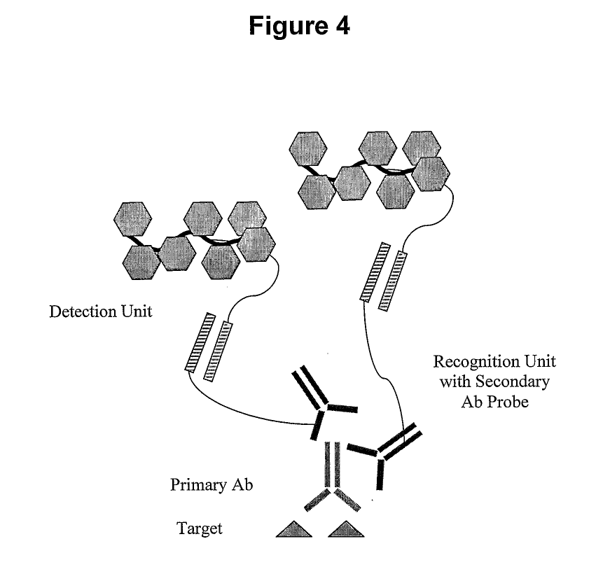

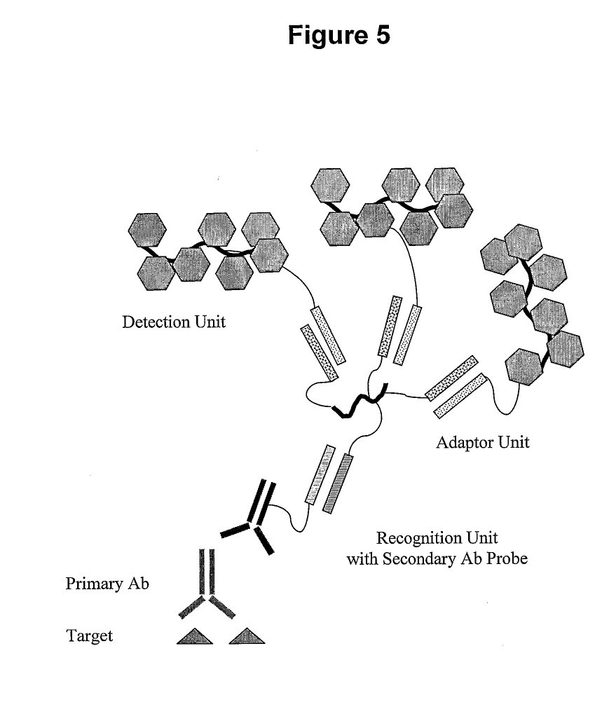

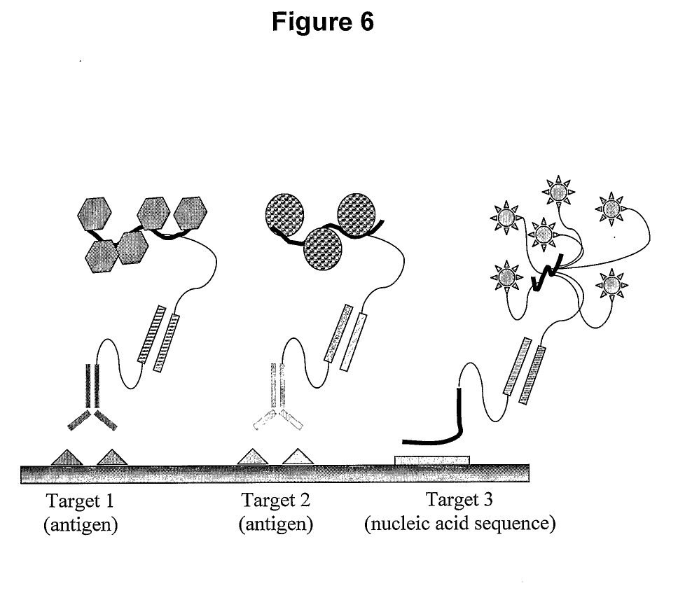

In some embodiments, the probes and detectable labels are comprised within larger molecular entities that either interact directly through covalently attached recognition elements such as hybridizing strands of nucleic acid or haptens, or that interact indirectly through one or more separate adaptor units. See FIGS. 3-6 for an example of such systems.

For example, the cross-linker may be added after the targets are probed by recognition units carrying the probes, in order to fix them in place. Then, buffer conditions may be altered such that the entities carrying the detectable labels, called detection units in FIG. 3, may bind directly or indirectly to the recognition units, in some embodiments using a different type of chemical reaction. In such a case, the chemical language of the target and probe binding is translated into that of the binding of the detection unit, optional adaptor unit, and recognition unit. To give an example, if the probe binds a target through protein-protein interactions, but the probe carries a nucleic acid strand that is used to hybridize to the detectable label, then the protein-protein target-probe interaction is translated into a base-pairing interaction.

Exemplary Recognition and Detection Units

In some embodiments, the recognition units and detection units are engineered such that they hybridize via nucleic acids or nucleic acid analog segments, haptens, antigens, and other entities present on those larger molecular units. Thus, one may optimize the conditions for detection based on the nucleic acid, hapten, antigen, etc., interactions rather than by the more limited set of direct interactions between the target and label. In such a way, the recognition and detection units “translate” the chemical language of the targets into that of nucleic acid hybridization or some other interaction.

Ordinarily two or more different targets would be recognized by different probes and detectable labels, resulting in different molecular interactions with the targets that may require different conditions for optimization. In contrast, using the recognition and detection units in connection with the instant cross-linking methods, in contrast, allows different targets to recognize different labels using the same set of molecular interactions and buffer conditions.

### Exemplary Adaptor Units

In order to allow for such intervening interactions, additional molecular entities called adaptor units may serve to link recognition units and detection units together. FIG. 1 b depicts an exemplary adaptor unit according to the invention, while other examples are illustrated in FIGS. 3, 5-12, 14-17, 21, 23, and throughout the application as a whole.

In some embodiments, the adaptor unit uses other recognition elements such as haptens, antigens, or binding agents in order to link the detection and recognition units together. In other embodiments, an adaptor unit has two nucleic acid analog segments, one to hybridize specifically to a recognition unit and another to hybridize specifically to a detection unit. In other embodiments, an adaptor unit has more than two nucleic acid analog segments or other recognition entities, either of the same or different type.

In some embodiments of the invention, the cross-linking may be conducted using a detection system comprising a recognition unit and a detection unit such that:
a) each unit comprises at least one nucleic acid analog segment;
b) at least one nucleic acid analog segment of the recognition unit specifically hybridizes to at least one nucleic acid analog segment of the detection unit;
c) the recognition unit further comprises at least one probe which recognizes at least one target in a sample;
d) the detection unit further comprises at least one detectable label; and
e) the nucleic acid analog segments on the recognition unit and detection unit that specifically hybridize to other nucleic acid analog segments on the recognition unit and detection unit do not specifically hybridize to the probe, detectable label, or target.
   See FIGS. 3-6 for examples.

In some embodiments, adaptors may function as “master keys” to connect one recognition unit to several different detector units, for instance, detection units with different kinds of detectable labels. Alternatively, adaptor units may link one detector unit to several different kinds of recognition units, and thus to several different kinds of probes. For example, when the units interact through nucleic acid hybridization, degenerately pairing nucleic acid sequences may be constructed such that one nucleic acid analog segment interacts with more than one complementary segment.

In other embodiments, adaptor units may also serve to enhance the signal from recognition of a target. For instance, an adaptor unit with several copies of the same nucleic acid analog segment may specifically hybridize to several detector units, thus increasing the number of detectable labels linked to a given target in a sample.

In certain embodiments, two or more of the recognition, adaptor, and detection units may be pre-hybridized or pre-bound prior to bringing the composition into contact with the sample.

Nucleic Acid Segments Used to Associate Recognition, Detection, and Adaptor Units

While one may envision many ways in which detection, recognition, and adaptor units can interact chemically, in some embodiments, recognition, detection, and optionally, adaptor units interact via nucleic acid hybridization. In some embodiments, nucleic acid analog segments are used. Nucleic acid analog segments present on the recognition, detection, and optional adaptor units may comprise at least one non-natural base and/or a non-natural backbone unit within the segment as a whole. Such non-natural units thus include, but are not limited to, for example, PNA's or phosphorothioate or 2′O-methyl nucleosides comprising the one of the natural bases A, C,G, T, or U, and, for example, natural RNA or DNA nucleosides comprising non-natural base such as 4-thio-Uracil or Inosine.

Non-natural bases may include, for example, purine-like and pyrimidine-like molecules, such as those that may interact using Watson-Crick-type, wobble, or Hoogsteen-type pairing interactions. Examples include generally any nucleobase referred to elsewhere as “non-natural” or as an “analog.”

Examples include: halogen-substituted bases, alkyl-substituted bases, hydroxy-substituted bases, and thiol-substituted bases, as well as 5-propynyl-uracil, 2-thio-5-propynyl-uracil, 5-methylcytosine, isoguanine, isocytosine, pseudoisocytosine, 4-thiouracil, 2-thiouracil and 2-thiothymine, inosine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

Yet other examples include bases in which one amino group with a hydrogen is substituted with a halogen (small “h” below), such as 2-amino-6-“h”-purines, 6-amino-2-“h”-purines, 6-oxo-2-“h”-purines, 2-oxo-4-“h”-pyrimidines, 2-oxo 6-“h”-purines, 4-oxo-2-“h”-pyrimidines. Those will form two hydrogen bond base pairs with non-thiolated and thiolated bases; respectively, 2,4 dioxo and 4-oxo-2-thioxo pyrimidines, 2,4 dioxo and 2-oxo-4-thioxo pyrimidines, 4-amino-2-oxo and 4-amino-2-thioxo pyrimidines, 6-oxo-2-amino and 6-thioxo-2-amino purines, 2-amino-4-oxo and 2-amino-4-thioxo pyrimidines, and 6-oxo-2-amino and 6-thioxo-2-amino purines.

For example, some specific embodiments of non-natural bases are the structures shown in FIG. 7 with the following substituents, which are described in the examples that follow as well as in the accompanying International Application entitled “New Nucleic Acid Base Pairs,” submitted herewith.

| Base | | | | | |
|---|---|---|---|---|---|
| (Symbol) | R2 | R3 | R4 | R5 | R6 |
| A | H or CH₃ | H | H | | |
| isoA | H or CH₃ | H | H | | |
| D | H or CH₃ | H | | | |
| G | H or CH₃ | H | | | |
| Gs | H or CH₃ | H | | | |
| I | H or CH₃ | | H | | |
| U | | | | H or CH₃ | |
| U2s | | | | H or CH₃ | |
| U4s | | | | H or CH₃ | |
| C | | | | H or CH₃ | H |
| Py-2o | | | H or CH₃ | H or CH₃ | |
| Cs | | | | H or CH₃ | H |
| isoG | H or CH₃ | H | | | |
| isoGs | H or CH₃ | H | | | |
| Pu-2o | H or CH₃ | | H | | |
| isoC | | H | | H or CH₃ | |
| isoCs | | H | | H or CH₃ | |
| Py-4o | | | H or CH₃ | H or CH₃ | |
| A | H or CH₃ | H | CH₃ | | |
| isoA | H or CH₃ | H | CH₃ | | |
| D | H or CH₃ | CH₃ | | | |
| G | H or CH₃ | CH₃ | | | |
| Gs | H or CH₃ | CH₃ | | | |
| I | H or CH₃ | | CH₃ | | |
| U | | | | H or CH₃ | |
| U2s | | | | H or CH₃ | |
| U4s | | | | H or CH₃ | |
| C | | | | H or CH₃ | CH₃ |
| Py-2o | | | H or CH₃ | H or CH₃ | |
| Cs | | | | H or CH₃ | CH₃ |
| isoG | H or CH₃ | CH₃ | | | |
| isoGs | H or CH₃ | CH₃ | | | |
| Pu-2o | H or CH₃ | | CH₃ | | |
| isoC | | CH₃ | | CH₃ or CH₃ | |
| isoCs | | CH₃ | | CH₃ or CH₃ | |
| Py-4o | | | H or CH₃ | H or CH₃ | |

In other examples, one or more of the H or CH₃ are independently substituted with a halogen such as Cl or F. FIG. 9 illustrates yet other exemplary bases and base pairs compatible with the instant invention. R₁ or “BB” in the structures of FIGS. 7-9 may serve as a point of attachment to a backbone group, such as PNA, DNA, RNA, etc.

In some embodiments, the following types of base pairs are used: one or more of Us:A, T:D, C:G, and P:Gs. In some embodiments, T:A and P:G are used. Still other examples are illustrated in FIGS. 2(A) and 2(B) of Buchardt et al. (U.S. Pat. No. 6,357,163).

Nucleic acid analog segments also include any oligomer, polymer, or polymer segment, comprising at least one monomer with a non-natural backbone unit: in other words, any backbone unit that is not a phosphoribo (RNA) or a phosphodeoxyribo (DNA) unit. Such non-natural backbone units include, but are not limited to, for example PNA's or phosphorothioate or 2′O-methyl backbones. For example, in some embodiments, one or more phosphate oxygens may be replaced by another molecule, such as sulfur. In other embodiments, a different sugar or a sugar analog may be used, for example, one in which a sugar oxygen is replaced by hydrogen or an amine, or an O-methyl. In yet other embodiments, nucleic acid analog segments comprise synthetic molecules that can bind to a nucleic acid or nucleic acid analog. For example, a nucleic acid analog may be comprised of peptide nucleic acids (PNAs), locked nucleic acids (LNAs), or any derivatized form of a nucleic acid. Such backbone units may be attached to any base, including the natural bases A, C, G, T, and U, and non-natural bases.

As used herein, “peptide nucleic acid” or “PNA” means any oligomer or polymer comprising at least one or more PNA subunits (residues), including, but not limited to, any of the oligomer or polymer segments referred to or claimed as peptide nucleic acids in U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470 6,201,103, 6,228,982 and 6,357,163; all of which are herein incorporated by reference.

The term PNA also applies to any oligomer or polymer segment comprising one or more subunits of the nucleic acid mimics described in the following publications: Lagriffoul et al., Bioorganic & Medicinal Chemistry Letters, 4: 1081-1082 (1994); Petersen et al., Bioorganic & Medicinal Chemistry Letters, 6: 793-796 (1996); Diderichsen et al., Tett. Lett. 37: 475-478 (1996); Fujii et al., Bioorg. Med. Chem. Lett. 7: 637-627 (1997); Jordan et al., Bioorg. Med. Chem. Lett. 7: 687-690 (1997); Krotz et al., Tett. Lett. 36: 6941-6944 (1995); Lagriffoul et al., Bioorg. Med. Chem. Lett. 4: 1081-1082 (1994); Diederichsen, U., Bioorganic & Medicinal Chemistry Letters, 7: 1743-1746 (1997); Lowe et al., J. Chem. Soc. Perkin Trans. 1, (1997) 1: 539-546; Lowe et al., J. Chem. Soc. Perkin Trans. 11: 547-554 (1997); Lowe et al., J. Chem. Soc. Perkin Trans. 1 1:5 55-560 (1997); Howarth et al., J. Org. Chem. 62: 5441-5450 (1997); Altmann, K-H et al., Bioorganic & Medicinal Chemistry Letters, 7: 1119-1122 (1997); Diederichsen, U., Bioorganic & Med. Chem. Lett., 8: 165-168 (1998); Diederichsen et al., Angew. Chem. Int. Ed., 37: 302-305 (1998); Cantin et al., Tett. Lett., 38: 4211-4214 (1997); Ciapetti et al., Tetrahedron, 53: 1167-1176 (1997); Lagriffoule et al., Chem. Eur. J., 3: 912-919 (1997); Kumar et al., Organic Letters 3(9): 1269-1272 (2001); and the Peptide-Based Nucleic Acid Mimics (PENAMs) of Shah et al. as disclosed in WO96/04000.

As used herein, the term “locked nucleic acid” or “LNA” means an oligomer or polymer comprising at least one or more LNA subunits. As used herein, the term “LNA subunit” means a ribonucleotide containing a methylene bridge that connects the 2′-oxygen of the ribose with the 4′-carbon. See generally, Kurreck, Eur. J. Biochem., 270:1628-44 (2003).

Nucleic acid segments may be synthesized chemically or produced recombinantly in cells (see e.g. Sambrook et al. (1989) Molecular Cloning: A Laboratory Manual, 2nd ed. Cold Spring Harbor Press). Methods of making PNAs and LNAs are also known in the art (see e.g. Nielson, 2001, Current Opinion in Biotechnology 12:16; Sorenson et al. 2003, Chem. Commun. 7(17):2130).

In certain embodiments, one or more of the recognition, adaptor, and detection units according to the invention comprise more than one nucleic acid analog segment. The two segments may have the same or different sequences.

Interactions between nucleic acid analog segments according to this invention may serve to link a recognition unit to a detector unit, either directly, or through the at least one optional adaptor unit. Different nucleic acid analog segments may hybridize, for instance, using Watson-Crick-type, wobble, or Hoogsteen-type base-pairing. Accordingly, the nucleic acid analog segments comprise sequences which allow for hybridization to take place at a desired stringency.

In some embodiments, the nucleic acid analog segments may pair specifically with more that one other nucleic acid analog segment, thereby providing degeneracy to the recognition, detection and/or adaptor units. See, for example, the International Application submitted herewith entitled “New Nucleic Acid Base Pairs,” and see the examples below.

This forms the basis for creating systems in which one nucleic acid analog segments may function as a “master-key” with the ability to hybridize to many partners, where each partner may also hybridize to separate nucleic acid analog segments. In that way, for example, a very versatile and flexible detection system may be constructed in some embodiments that allows the user to choose between visualizing several targets via different detectable labels and detection units, or via only one detectable label and detection unit.

### Hybridization of Nucleic Acids and Nucleic Acid Analog Segments

Two different nucleic acid analog segments on the recognition unit, detection unit, and/or adaptor unit may specifically hybridize. In some embodiments, the chosen hybridization conditions are “stringent conditions,” meaning herein conditions for hybridization and washes under which nucleotide sequences that are significantly complementary to each other remain bound to each other. The conditions are such that sequences at least 70%, at least 80%, at least 85-90% complementary remain bound to each other. The percent complementary is determined as described in Altschul et al. (1997) Nucleic Acids Res. 25:3389-3402 (hereby incorporated by reference).

Specified conditions of stringency are known in the art and can be found in Current Protocols in Molecular Biology, John Wiley & Sons, Inc. (Ausubel et al. 1995 eds.), sections 2, 4, and 6 (hereby incorporated by reference). Additionally, specified stringent conditions are described in Sambrook et al. (1989) Molecular Cloning: A Laboratory Manual, 2nd ed. Cold Spring Harbor Press, chapters 7, 9, and 11 (hereby incorporated by reference).

In other embodiments, the chosen hybridization conditions are “high stringency conditions.” An example of high stringency hybridization conditions is hybridization in 4× sodium chloride/sodium citrate (SSC) at 65-70° C. or hybridization in 4×SSC plus 50% formamide at 42-50° C., followed by one or more washes in 1×SSC, at 65-70° C. It will be understood that additional reagents may be added to hybridization and/or wash buffers, e.g., blocking agents (BSA or salmon sperm DNA), detergents (SDS), chelating agents (EDTA), Ficoll, PVP, etc.

In yet other embodiments, the chosen conditions are “moderately stringent conditions.” Moderate stringency, as used herein, includes conditions that can be readily determined by those having ordinary skill in the art based on, for example, the length of the nucleic acid analog segment. Exemplified conditions are set forth by Sambrook et al. Molecular Cloning: A Laboratory Manual, 2d ed. Vol. 1, pp. 1.101-104, Cold Spring Harbor Laboratory Press (1989) (hereby incorporated by reference), and include use of a prewashing solution of 5X SSC, 0.5% SDS, 1.0 mM EDTA (pH 8.0), hybridization conditions of 50% formamide, 6×SSC at 42° C. (or other similar hybridization solution, such as Stark's solution, in 50% formamide at 42° C.), and washing conditions of 60° C., 0.5×SSC, 0.1% SDS.

In some embodiments, the chosen conditions are “low stringency” conditions. Low stringency conditions may include, as used herein, conditions that can be readily determined by those having ordinary skill in the art based on, for example, the length of the nucleic acid analog segment. Low stringency may include, for example, pretreating the segment for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% W/V dextran sulfate, and 5-20×10⁶ CPM probe is used. Samples are incubated in hybridization mixture for 18-20 hours at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C.

These different sets of hybridization conditions may also be used when a nucleic acid segment or nucleic acid analog segment is used as a binding agent, a probe, or a detection label.

### Polymers

One or more of the recognition unit, detection unit, and adaptor unit may also comprise at least one polymer. A “polymer,” as used herein, may be any molecule that facilitates covalent or non-covalent attachment of one or more other components of a recognition unit, detection unit, and/or adaptor unit. For instance, the polymer may facilitate the attachment of one or more probes, nucleic acid analog segments, and or detectable labels. The polymer may be a soluble molecule or an insoluble molecule and may have any shape including a linear polymer, branched polymer, bead or other globular shaped polymer.

Examples of suitable polymers include polysaccharides such as dextrans, carboxy methyl dextran, dextran polyaldehyde, carboxymethyl dextran lactone, and cyclodextrins; pullulans, schizophyllan, scleroglucan, xanthan, gellan, O-ethylamino guaran, chitins and chitosans such as 6-O-carboxymethyl chitin and N-carboxymethyl chitosan; derivatized cellolosics such as carboxymethyl cellulose, carboxymethyl hydroxyethyl cellulose, hydroxyethyl cellulose, 6-amino-6-deoxy cellulose and O-ethylamine cellulose; hydroxylated starch, hydroxypropyl starch, hydroxyethyl starch, carrageenans, alginates, and agarose; synthetic polysaccharides such as ficoll and carboxymethylated ficoll; vinyl polymers including poly(acrylic acid), poly(acryl amides), poly(acrylic esters), poly(2-hydroxy ethyl methacrylate), poly(methyl methacrylate), poly(maleic acid), poly(maleic anhydride), poly(acrylamide), poly(ethyl-co-vinyl acetate), poly(methacrylic acid), poly(vinylalcohol), poly(vinyl alcohol-co-vinyl chioroacetate), aminated poly(vinyl alcohol), and co block polymers thereof; poly ethylene glycol (PEG) or polypropylene glycol or poly(ethylene oxide-co-propylene oxides) containing polymer backbones including linear, comb-shaped or hyperbranched polymers and dendrimers, including branched PAMAM-dendrimers; poly amino acids including polylysines, polyglutamic acid, polyurethanes, poly(ethylene imines), pluriol; proteins including albumins, immunoglobulins, and virus-like proteins (VLP), and polynucleotides, DNA, PNA, LNA, oligonucleotides and oligonucleotide dendrimer constructs. Also contemplated is the use of mixed polymers, i.e., a polymer comprised of one or more of the above examples including any of the polymers, the co-block polymers and random co-polymers.

Properties of the polymer can be varied, depending on the desired application, to optimize performance. Examples of parameters that may be considered in the choice of a polymer include the length of the polymer and branching of the polymer. Furthermore, the polymer may carry various substituents. The substituents may be chemically protected and/or activated, allowing the polymer to be derivatized further.

### Linkers

The recognition units, detection units, and adaptor units of the present invention may also comprise one or more linkers, for example, placed between the probe and the recognition element. A “linker,” as used herein, is a molecule that may help to join other atoms, molecules, or functional groups together through chemical bonds. In the instant applications for example, a linker may serve to join various components of each of the units together, such as probes, nucleic acid analog segments, polymers, and detectable labels.

In some embodiments, the linker also is of sufficient length or size such that the various parts, though chemically attached together, nonetheless remain separated from each other in space, thus minimizing steric clashes. For instance, a linker on a recognition unit may serve to join a probe to a nucleic acid analog segment, while separating them sufficiently to avoid steric clashes. A linker may also serve to separate a polymer from another component of one of the units such as a nucleic acid analog segment, to separate two or more nucleic acid analog segments, or to separate a detectable label from a nucleic acid analog segment, or to separate multiple probes or multiple detectable labels. Linkers may also increase the solubility of the conjugates and may prevent unwanted interactions by shielding the components and may thereby confer a general and significant lower non-specific background for the visualization system.

Reducing the steric hindrance between the various components of the different units of the composition may also improve detection efficiency. For example, certain detection labels show reduced signals when in close proximity to other detection labels. Fluorescent labels, for instance, may become quenched if present in close proximity. Further, reducing steric hindrance increases the binding affinity of the various components for their intended binding partners and decreases the level of the background and the risk of false positive signals.

A person of ordinary skill in the art of molecular conjugation knows numerous linkers. Examples include 6-amino-hexanoic acid, succimidyl 4-(N-malemidomethyl)cylohexane-1-carboxylate (SMCC), homobifunctional linkers such as divinyl sulfone (DVS), glutaric dialdehyde, hexane di-isocyanate, dimethylapimidate, 1,5-difluoro-2,4-dinitrobenzene, heterobifunctional linkers like e.g. N-gamma-maleimidobytyroloxy succinimide ester (GMBS), and zero length linkers such as 1-ethyl-3-(3-dimethylaminopropyl)cabodiimide

Longer linker molecules based upon polyethylene glycol (PEG) are also available in the art. (See, for example, Discrete PEG (dPEG)™ modification reagents available from Quanta Biodesign, Ltd., Powell, Ohio, or at www.quantabiodesign.com; PEG-based reagents available from EMD Biosciences, Inc., San Diego, Calif., described in Novabiochem April, 2004, “Product focus: PEG reagents—bifunctional amino-PEG-acid spacers” brochure, available at www.novabiochem.com; and see Baumeister et al., Biopolymers, 71: 339 (2003); Kumar & Aldrich, Org. Lett., 5: 613 (2003). (See also, “Chemistry of Protein Conjugation and Cross-Linking” Shan S. Wong CRC Press, Boca Raton, Fla., USA, 1993; “BioConjugate Techniques” Greg T. Hermanson Academic Press, San Diego, Calif., USA, 1996; “Catalog of Polyethylene Glycol and Derivatives for Advanced PEGylation, 2004” Nektar Therapeutics Inc, Huntsville, Ala., USA).

The present invention may also use a long uncharged linker comprising at least two units of the Formula I.

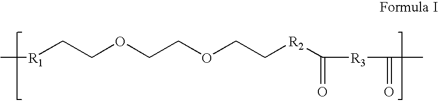

In Formula I, R₁ and R₂ may comprise either NH or O, while R₃ may comprise methyl, ethyl, propyl, CH₂—O—CH₂, and (CH₂—O—CH₂)₂. For example, in some embodiments of the instant invention, the linker comprises at least two units of the Formula I wherein R₁ and R₂ are both NH and R₃ is CH₂—O—CH₂. See the examples that follow and the accompanying International Application entitled “MONOMERIC AND POLYMERIC LINKERS USEFUL FOR CONJUGATING

BIOLOGICAL MOLECULES AND OTHER SUBSTANCES” for further description of this linker.

### Methods of Making Detection System Units

The recognition units, detection units, and adaptor units according to the invention may comprise, for example, molecules in which the various components such as probes, detection labels, nucleic acid analog segments and other binding entities, linkers, and polymers are covalently attached to form conjugates. As used herein, the terms “conjugate, conjugation” and the like refer to the formation of covalent attachments between various substances, either directly without intervening bonds, or indirectly through at least one intervening bond. Alternatively, in some embodiments, the components of each unit may be attached through stable, non-covalent interactions such as base-pairing, adsorption, intercalation, and similar hydrogen bonding, van der Waals, or hydrophobic interactions, that are sufficiently stable under conditions of use.

Many methods of conjugating molecules are known in the art and can be used to make the various units of the invention. For example, conjugates comprising a linker or polymer according to this invention may be formed by covalently coupling amino groups to conjugated double bonds on a polymer or linker. In one embodiment the polymer is activated with divinylsulfone and mixed with a probe, nucleic acid analog segment, and/or detectable label to form a polymer conjugate. In other embodiments, aldehydes may be used to activate a polymeric backbone. For instance, dextrans may then be mixed with the binding agent and an optional detectable label. Yet another method of preparing polymeric conjugates is by using so called chemo-selective schemes for coupling the components together, e.g., enzymes or other molecules can be derivatized with thiol-reactive maleimide groups before being conjugated to a thiol-modified polymeric carrier or backbone.

In some embodiments no exogenous polymeric backbone is required for attachment of a probe, detectable label, and/or nucleic acid analog segment to one of the instant units. In these embodiments, the components themselves may be activated for conjugation or may be self-polymerizable. For example, a vinyl group may be used to activate the components for conjugation. Polymerization then occurs by addition of a radical, which results in polymerization of the vinyl groups to form a polymeric conjugate. The conjugate thus will contain a poly vinyl backbone or blocks of poly vinyl. Alternatively, active esters of acrylic acid can be used to activate proteins and other molecules. Generation of free radicals can polymerize the derivatized molecules. Small molecule linkers with more than one vinyl group can be further added to help form a polymeric conjugate.

In some embodiments, the components may be organized in the unit with the help of one or more linkers, as described above. Many such linkers are known in the art and available commercially, as described, and the linkers may be activated for attachment to other components of the units according to the invention according to methods available from commercial suppliers or in the literature. See the working examples that follow and the accompanying International Application entitled “MONOMERIC AND POLYMERIC LINKERS USEFUL FOR CONJUGATING BIOLOGICAL MOLECULES AND OTHER SUBSTANCES,” for examples.

### Working Examples

### Example 1

### Detecting Gene and Protein Targets in the Same Sample

### Part A.

The sample is denatured and hybridized to a nucleic acid or nucleic acid analog probe by first incubating at 95° C. for a period of time, then at 40-60° C. Then the sample is incubated with an optionally labeled primary antibody probe specific for a protein target in the sample. A cross-linking agent is added. Following cross-linking, the sample is incubated with one or more detection conjugates, including a labeled secondary antibody if the primary antibody is not directly labeled, and the protein and gene targets are visualized via a color or fluorescent signal.

### Part B.

The sample is denatured and hybridized to a nucleic acid or nucleic acid analog probe by first incubating at 95° C. for a period of time, then at 40-60° C. Then the sample is incubated with an optionally labeled primary antibody probe specific for a protein target in the sample. A cross-linking agent is added. The sample is next incubated with one or more adaptor molecules that recognize the primary antibody and nucleic acid or nucleic acid analog probe. The adaptor molecules contain one or more additional recognition elements that will recognize the detection reagents to be added in a later step. The sample is again exposed to a cross-linking agent to cross-linke the adaptor molecules to components of the sample. The sample is incubated with detection conjugates and the targets are detected by color or fluorescent signals.

### Part C.

The sample is denatured incubating at 95° C. for a period of time. Then the sample is incubated with an optionally labeled primary antibody probe specific for a protein target in the sample. The sample is next incubated with one or more adaptor molecules that recognizes the primary antibody. The adaptor molecules contain one or more additional recognition elements that will recognize the detection reagents to be added in a later step. A cross-linking agent is added, which cross-links the adaptor molecules to other elements of the sample. Following cross-linking, the sample is heated to retrieve a nucleic acid target in the sample, cooled, and a nucleic acid or nucleic acid probe is added. The sample is optionally further exposed to a cross-linking agent. Then, the sample is incubated with one or more detection conjugates, and the protein and gene targets are visualized via a color or fluorescent signal.

### Part D.

The sample is denatured incubating at 95° C. for a period of time. Then the sample is incubated with an optionally labeled primary antibody probe specific for a protein target in the sample, and appropriate washing is carried out. Optionally, the sample is next incubated with one or more adaptor molecules that recognizes the primary antibody. The adaptor molecules contain one or more additional recognition elements that will recognize the detection reagents to be added in a later step. A cross-linking agent is added, which cross-links the adaptor molecules to other elements of the sample, and the sample is washed again. Then, the sample is incubated with one or more detection conjugates, and the protein target is visualized via a color or fluorescent signal.

### Example 2

### Detection of Two or More Protein Targets in the Same Sample

A sample is subjected to target retrieval by heating at 90-100° C. for 20-40 minutes, and then cooled. A mixture or two or more optionally labeled primary antibody probes are then added, and the sample exposed to a cross-linking agent. The sample is next incubated with one or more adaptor molecules that recognizes the primary antibody. The adaptor molecules contain one or more additional recognition elements that will recognize the detection reagents to be added in a later step. The system is again cross-linked. Detection conjugates are added and the targets are visualized by color or fluorescent labels.

### Example 3a

### Preparation of Pyrimidinone-Monomer

1. In dry equipment 4.6 g of solid Na in small pieces was added to 400 mL ethanol (99.9%), and was dissolved by stirring. Hydroxypyrimidine hydrochloride, 13.2 g, was added and the mixture refluxed for 10 minutes. Then 12.2 mL ethyl-bromoacetate (98%) was added and the mixture refluxed for 1½ hour. The reaction was followed using Thin Layer Chromatography (TLC). The ethanol was evaporated leaving a white compound, which was dissolved in a mixture of 80 mL of 1M NaCitrate (pH 4.5) and 40 mL of 2M NaOH. This solution was extracted four times with 100 mL Dichloromethane (DCM). The DCM phases were pooled and washed with 10 mL NaCitrate/NaOH—mixture. The washed DCM phases were evaporated under reduced pressure and resulted in 17.2 g of crude solid product. This crude solid product was recrystallized with ethylacetate giving a yellow powder. The yield for this step was 11.45 g (63%).

2. The yellow powder, 12.45 g. from above was hydrolyzed by refluxing overnight in a mixture of 36 mL DIPEA, 72 mL water and 72 mL dioxane. The solvent was evaporated and water was removed from the residue by evaporation from toluene. The yield for this step was 100%.

3. OBS. Pyrimidinone acetic acid (10.5 g), 16.8 g PNA-backbone ethylester, 12.3 g DHBT-OH, 19 mL Triethylamine was dissolved in 50 mL N,N-dimethylformamide (DMF). DIPIDIC (11.8 mL) was added and the mixture stirred overnight at room temperature. The product was taken up in 100 mL DCM and extracted three times with 100 mL of dilute aqueous NaHCO₃. The organic phase was extracted twice with a mixture of 80 mL of 1M NaCitrate and 20 mL of 4M HCl. Because TLC showed that some material was in the citrate phase, it was extracted twice with DCM. The organic phases were pooled and evaporated. Because there was a precipitation of urea, the product was dissolved in a DCM, and the urea filtered off. Subsequent evaporation left an orange oil. Purification of the orange oil was performed on a silica column with 10% methanol in DCM. The fractions were collected and evaporated giving a yellow foam. The yield for this step was 7.0 g (26.8%).

4. The yellow foam (8.0 g) was hydrolyzed by reflux overnight in 11 mL DIPEA, 22 mL water, and 22 mL dioxane. The solvent was evaporated and the oil was dehydrated by evaporation from toluene leaving an orange foam. The yield for this step was 100%.

### Example 3b

### Second Method of Preparing Pyrimidonone Monomer

Step 1. In dry equipment 9.2 g of solid Na in small pieces was dissolved in 400 mL ethanol (99.9%), with stirring. Hydroxypyrimidine hydrochlorid, 26.5 g, was added, and the mixture was stirred for 10 minutes at 50° C. Then 24.4 mL Ethyl bromoacetate (98%) was added and the mixture stirred at 50° C. for 1 hour. The reaction was followed using Thin Layer Chromatography (TLC).

The ethanol was evaporated leaving a white compound, which was dissolved in 70 mL of water and extracted with 20 mL DCM. Another 30 mL of water was added to the water phase, which was extracted with 3×100 mL DCM. The DCM-phase from the first extraction contains a lot of product, but also some impurities, wherefore this phase was extracted twice with water. These two water phases then were back extracted with DCM.

The combined DCM phases were pooled and washed with 10 mL water. The washed DCM phases were evaporation under reduced pressure and resulted in 25.1 g yellow powder. The yield for this step was 25.1 g=69%. Maldi-Tof: 181.7 (calc. 182).

Step 2. 34.86 g yellow powder from above was dissolved in 144 mL 2M NaOH. After stirring 10 minutes at room temperature, the mixture was cooled in an ice bath. Now 72 mL 4 M HCl (cold) was added. The product precipitated. After stirring for 5 minutes, the precipitate was filtered and thoroughly washed with ice water. Drying in a dessicator under reduced pressure left 18.98 g yellow powder. The yield for this step was 18.98 g=64%.

Step 3. Pyrimidinone acetic acid 11.1 g and triethylamine 12.5 mL were dissolved in N,N-dimethylformamide (DMF) 24 ml, HBTU 26.2 g was added plus 6 mL extra DMF. After 2 minutes a solution of PNA-Backbone ethylester 14.7 g dissolved in 15 mL DMF was added. The reaction mixture was stirred at room temperature and followed using TLC. After 1½ hour precipitate had formed. This was filtered off.

The product was taken up in 100 mL DCM and extracted with 2×100 mL dilute aqueous NaHCO3. Both of the aqueous phases were washed with a little DCM. The organic phases were pooled and evaporated. Evaporation left an orange oil. Purification of the product was done on a silica column with 10-20% methanol in ethylacetate. The fractions were collected and evaporated giving a yellow oil. The oil was dissolved and evaporated twice from ethanol. The yield from this step was 20.68 g =90%.

Step 4. The yellow oil (18.75 g) was dissolved in 368 mL 0.2 M Ba(OH)2. Stirring for 10 minutes before 333 mL 0.221 M H2SO4 was added. A precipitation was performed immediately. Filtration through cellite, which was washed with water. The solvent was evaporated. Before the evaporation was at end, the product was centrifuged to get rid of the very rest of the precipitation. Re-evaporation of the solvent left a yellow oil. The yield from this step was 13.56 g=78%.

Step 5. To make a test on the P-monomer 3 consecutive P's were coupled to Boc-L300-Lys(Fmoc)-resin, following normal PNA standard procedure. The product was cleaved from the resin and precipitated also following standard procedures: HPPP-L300-Lys(Fmoc). Maldi-Tof on the crude product: 6000 (calc. 6000) showing only minor impurities.

### Example 4

### Preparation of the Thio-Guanine Monomer

1. 6-Chloroguanine (4.93 g) and 10.05 g K₂CO₃ was stirred with 40 mL DMF for 10 minutes at room temperature. The reaction mixture was placed in a water bath at room temperature and 3.55 mL ethyl bromoacetate was added. The mixture was stirred in a water bath until TLC (20% Methanol/DCM) showed that the reaction was finished. The precipitated carbonate was filtered off and washed twice with 10 mL DMF. The solution, which was a little cloudy, was added to 300 ml water, whereby it became clear. On an ice bath the target compound slowly precipitated. After filtration the crystals were washed with cold ethanol and dried in a desiccator. The yield for this step was 3.3 g (44.3%) of ethyl chloroguanine acetate.

2. Ethyl chloroguanine acetate (3.3 g) was dissolved by reflux in 50 mL absolute ethanol. Thiourea (1.08 g) was added. After a refluxing for a short time, precipitate slowly began forming. According to TLC (20% Methanol/DCM) the reaction was finished in 45 minutes. Upon completion, the mixture was cooled on an ice bath. The precipitate was then filtered and dried overnight in a desiccator. The yield for this step was 2.0 g (60%) ethyl thioguanine acetate.

3. Ethyl thioguanine acetate (3.57 g) was dissolved in 42 mL DMF. Benzylbromide (2.46 mL) was then added and the mixture stirred in an oil bath at 45° C. The reaction was followed using TLC (25% Methanol/DCM). After 3 hours all basis material was consumed. The step 3 target compound precipitated upon evaporation under reduced pressure and high temperature. The precipitate was recrystallized in absolute ethanol, filtered and then dried in a desiccator. The yield for this step was 3.88 g (82%) of methyl benzyl thioguanine ethylester.

4. Methyl benzyl thioguanine ethylester (5.68 g) was dissolved in 12.4 mL of 2M NaOH and 40 mL THF, and then stirred for 10 minutes. The THF was evaporated by. This was repeated. The material was dissolved in water and then 6.2 mL of 4M HCl was added, whereby the target product precipitated. Filtering and drying in a desiccator. The yield for this step was 4.02 g (77%).

5. The product of step 4 (4.02 g), 3.45 g backboneethylester, 9 mL DMF, 3 mL pyridine, 2.1 mL triethylamine and 7.28 g PyBop were mixed and then stirred at room temperature. After 90 minutes a solid precipitation formed. The product was taken up in 125 mL DCM and 25 mL methanol. This solution was then extracted, first with a mixture of 80 mL of 1M NaCitrate and 20 mL of 4M HCl, and then with 100 mL dilute aqueous NaHCO₃. Evaporation of the organic phase gave a solid material. The material was dissolved in 175 mL boiling ethanol. The volume of the solution was reduced to about 100 mL by boiling. Upon cooling in an ice bath, the target product precipitate. The crystals were filtered, washed with cold ethanol and then dried in a desiccator. The yield of this step was 6.0 g (86%.)

6. The product of step 5 (6.0 g) was dissolved in 80 mL THF, 7.5 mL 2M NaOH and 25 mL water. The solution became clear after ten minutes of stirring. THF was evaporated. Water (50 mL) was added to the mixture. THF was evaporated. Water (50 mL) was added to the mixture. When the pH was adjusted by the addition of 3.75 mL of 4M HCl, thio-guanine monomer precipitated. It was then filtered, washed with water and dried in a desiccator. The yield for this step was 5.15 g (91%).

### Example 5

### Preparation of Diaminopurine Acetic Acid Ethyl Ester

1. Diaminopurine (10 g) and 40 g of K₂CO₃ were added to 85 mL of DMF and stirred for 30 minutes. The mixture was cooled in a water bath to 15° C. Ethyl bromoacetate (3 mL) was added three times with 20 minute intervals between each addition. This mixture was then stirred for 20 minutes at 15° C. The mixture was left in the water bath for another 75 minutes, and the temperature increased to 18° C. The DMF was removed by filtering and the remaining K₂CO₃ was added to 100 mL of ethanol and refluxed for 5 minutes. Filtering and repeated reflux of the K₂CO₃ in 50 mL ethanol, filtering. The pooled ethanol phases were placed in a freezer, after which crystals formed. These crystals were filtered, washed with cold ethanol, filtered again and then dried in a desiccator overnight. The yield for this step was 12 g (76%).

### Example 6

### Preparation of L₃₀-Linker

1. A solution of 146 mL of 2,2′-(Ethylenedioxy)bis(ethylamine) (98%) in 360 mL of THF was cooled in an ice bath. Di-tert-butyl dicarbonate (97%) (65 g) in 260 mL THF was added dropwise over one hour. The solvent was evaporated. The remaining oil was dissolved in water and then evaporated off. The oily product was dissolved in 300 mL water, extracted with 300 mL DCM, then washed twice with 150 mL of DCM. The collected organic phase was washed with 50 mL of water before evaporating to about half the volume. The organic phase was then extracted with 400 mL of 1M NaCitrate (pH 4.5), and then extracted again with 50 mL of 1M NaCitrate (pH 4.5). The aqueous phases were washed with 50 mL DCM before cooling on an ice bath. While stirring, 100 mL of 10M NaOH was added to the aqueous washed aqueous phases resulting in pH of 13-14. In a separation funnel the product separated on its own. It was shaken with 300 mL DCM and 50 ml water. The organic phase was evaporated, yielding a white oil. The yield for this step was 48.9 g (65.7%). The product had a predicted molecular formula of C₁₁H₂₄N₂O₄ (MW 248.3).

2. Boc-amine (76.2 g) was dissolved in 155 mL pyridine. Diglycolic anhydride (54.0 g) (90%) was added. After stirring for 15 minutes the intermediate product separated out and then 117 mL Acetic Anhydride (min. 98%) was added and the mixture stirred at 95° C. for 1 hour. The solution was then put under reduced pressure and evaporated. Water (117 mL) was added, and the mixture was then stirred for 15 minutes, after which 272 mL of water and 193 mL of DCM were added.

The organic layer was extracted twice with 193 mL of 1M Na₂CO₃ and then twice with a mixture of 72 mL of 4M HCl and 289 mL of 1M NaCitrate. After each extraction the aqueous phase was washed with a little DCM. The collected organic phase was washed with 150 mL of water. The solvent was evaporated leaving the product as an orange oil. This yield for this step was 100.3 g (0.29 mol) (94%). The product had a predicted molecular formula of C₁₅H₂₆N₂O₇ (MW 346.4).

3. The product from step 2 (100.3 g) was dissolved in an equal amount of THF and was then added dropwise to 169.4 mL of 2,2′-(Ethylendioxy)bis(ethylamine) at 60° C. over the period of 1 hour. The amine was distilled from the reaction mixture at 75-80° C. and a pressure of 3x10⁻¹ mBar. The residue from the distillation was taken up in a mixture of 88 mL of 4M HCl and 350 mL of 1M NaCitrate and then extracted three times with 175 mL of DCM. The aqueous phase was cooled in an ice bath and was cautiously added to 105 mL of 10M NaOH while stirring. In a separation funnel the product slowly separated from the solution. When separated 100 mL of water and 950 mL of DCM were added to the product. Stirring for some minutes before pouring to a separation funnel. The pH in the aqueous phase should be 14. The aqueous phase was extracted four times with 150 mL of DCM. The solvent was evaporated. The oily residue was dehydrated by evaporation from toluene, giving a yellow oil. The yield for this step was 115.48 g (81%). The product had a predicted molecular formula of C₂₁H₄₂N₄O₉ (MW 494.6).

4. The Boc-amine (115.48 g) from step 3 was dissolved in 115 mL of pyridine. Diglycolic anhydride (40.6 g) (90%) was added and the mixture stirred for 15 minutes, after which the intermediate product came out. Acetic Anhydride (97 mL) (min. 98%) was added and the mixture stirred at 95° C. for 1 hour. The mixture was then evaporated under reduced pressure. The mixture was then cooled and then 80 mL of water was added. This mixture was stirred for 15 minutes and then 200 mL of water and 150 mL of DCM were added. The organic layer was extracted twice with 150 mL of 1M Na₂CO₃ and then twice with a mixture of 53 mL of 4M HCl and 213 mL of 1M NaCitrate. After each extraction the aqueous phase was washed with a little DCM. The collected organic phase was washed with 150 mL of water. The solvent was evaporated. The oily residue was dehydrated by evaporation from toluene, giving a yellow oil. The yield for this step was 125 g (92%). The product had a predicted molecular formula of C₂₅H₄₄N₄O₁₂ (MW 592.6), with a mass spectrometry determined molecular weight of 492.5.

Further purifying of the product could be done on a silica column with a gradient from 5-10% methanol in DCM. The yield from the column purification was 69% and produced a white oil.

5. White oil (12.4 g) from step 4 was dissolved in a mixture of 12 mL water and 12 mL 1,4-Dioxane (99%) and was then heated to reflux. DIPEA (6 mL) was added and refluxed for 30 minutes. This mixture was cooled and then evaporated. The oily residue was dehydrated by evaporation from toluene, giving a yellow oil. The product had a predicted molecular formula of C₂₅H₄₆N₄O₁₄ (MW 610.6).

### Example 7

### Exemplary embodiments of PNA Sequences

All are made by PNA standard procedures (see Examples 17 and 18).

TABLE 1

| SEQUENCE | | N- | C- | MOLECULAR |
|---|---|---|---|---|
| DESIGNATION | PNA SEQUENCES¹ | TERMINAL | TERMINAL | WEIGHT |
| SEQ. AA | TCD-DG_{S}G_{S}-TAC-A | FLU-L₃₀- | -LYS(CYS) | 8805 |
| SEQ. AB | U_{S}GU_{S}-DPP-TTG-D | FLU-L₃₀- | -LYS(CYS) | 8727 |
| SEQ. AC | CU_{S}G_{S}-G_{S}DD-TU_{S}D-G_{S}DC | FLU-L₃₀- | -LYS(CYS) | 9413 |
| SEQ. AD | GTP-TAA-TTP-PAG | FLU-L₃₀- | -LYS(CYS) | 9203 |
| SEQ. AE | DG_{S}T-CG_{S}D-DG_{S}-G-U_{S}CU_{S} | FLU-L₃₀- | -LYS(CYS) | 9413 |
| SEQ. AF | AGA-CPT-TPG-APT | FLU-L₃₀- | -LYS(CYS) | 9187 |
| SEQ. AG | TCD-DI I-TAC-A | FLU-L₃₀- | -LYS(CYS) | 8742 |

¹Flu is fluorescein; T is thiamine; C is cytosine; D is diaminopurine; G_{S} is thioguanine; A is Adenine; U_{S} is 2/4-thiouracil; G is guanine; P is pyrimidone; I is inosine.

### Example 8

### Three PNAs with the L₃₀ Linker with Different Amino Acids at the C-Terminal

BA:
Flu-L₃₀-DGT-DTC-GTD-CCG-Lys(Acetyl)
BB:
Flu-L₃₀-DGT-DTC-GTD-CCG-Lys(Cys)
BC:
Flu-L₃₀-DGT-DTC-GTD-CCG-Lys(Lys)₃

### Example 9

### Synthesis of Flu-L₉₀-Lys(Flu)-L₃₀-Lys(Cys)

Using procedure provided in Example 18a, an MBHA-resin was loaded with Boc-Lys(Dde)-OH. Using a peptide synthesizer, amino acids were coupled according to PNA solid phase procedure provided in Example 18d yielding Boc-L₉₀-Lys(Fmoc)-L₃₀-Lys(Dde). The Boc and Fmoc protections groups were removed and the amino groups marked with flourescein using the procedure in Example 18e. Then, the Dde protection group was removed and 0.4 M cysteine was added according to the procedure in Example 18b. The PNA was cleaved from the resin, precipitated with ether and purified on HPLC according to Example 18d. The product was found to have a molecular weight of 3062 using MALDI-TOF mass spectrometry; the calculated molecular weight is 3061.

### Example 10

### Synthesis of a Conjugate Made from Sequence AA from Example 5, DexVS70, and Flu(10)

Dextran (with a molecular weight of 70 kDa) was activated with divinylsulfone to a degree of 92 reactive groups/dextran polymer; this product is designated DexVS70.

| | |
|---|---|
| 280 μL DexVS70 | 20 nmol |
| 66 μL Flu₂Cys | 160 nmol (prepared from Example 7) |
| 25 μL 0.8 M NaHCO₃ pH = 9.5 | |
| 29 μL H₂O | |

The above four compounds were mixed. The mixture was placed in a water bath at 30° C. for 16 hours. The mixture was added to 50 nmol of freeze-dried PNA (sequence AA from Example 5). The mixture was placed in a water bath at 30° C. for 30 minutes. The conjugating reaction was quenched with 50 μL of 500 mM cysteine for 30 minutes at 30° C. Purification of the product was performed using FPLC: column SUPERDEX®—200, buffer 10 mM Hepes 100 mM NaCl, method 7 bank 2, Loop 1 mL. Two fractions were collected: one with the product and one with the residue. The relative absorbance Flu₂ (ε_{500 nm}=146000 M⁻¹, ε_{260 nm}=43350 M⁻¹) and PNA (ε_{500 nm}=73000 M⁻¹, ε_{260 nm}=104000 M⁻¹) was used to calculate the average conjugation ratio of Flu₂, PNA, and DexVS70. The conjugation ratio of Flu₂ to DexVS70 was 9.4. The conjugation ratio of PNA (sequence AA) to DexVS70 was 1.2.

### Example 11

### Synthesis of HRP-DexVS70-Seq. AA

Using the procedure of Example 14, the conjugate HRP-DexVS70-Seq. AA was made. The ratio of HRP to DexVS70 is 12.2; the ratio of Seq. AA to Dex70 is 1.2.

### Example 12

### Synthesis of GaM-DexVS70-Seq. AB

The synthesis of GaM-DexVS70-Seq. AB was performed using the procedure in Example 16 with the following changes as indicated.

Dextran (molecular weight 70 kDa) is activated with divinylsulfone to a degree of 92 reactive groups/dextran polymer.

| | |
|---|---|
| 105.0 μL DexVS70 | 7.5 nmol |
| 57.0 μL Goat anti mouse Imuno globuline (GAM-Ig) | 15 nmol |
| 8.9 μL 4 M NaCl | |
| 10.6 μL 0.8 M NaHCO₃ (pH = 9.5) | |
| 144.5 μL H₂O | |

The above five components were mixed and placed in a water bath at 30° C. for 40 minutes. Two hundred and ninety μL were taken out of the mixture and added to 100 nmol of Seq. AB, which was previously dissolved in 80 μL of H₂O. Then, 20 μL of 0.8 M NaHCO₃ (pH 9.5) was added and the mixture placed in a water bath at 30° C. for 1 hour. Quenching was performed by adding 39 μL of 500 mM cysteine and letting the resultant mixture set for 30 minutes at 30° C.

Purification of the product on FPLC: column SUPERDEX®—200, buffer 10 mM Hepes 100 mM NaCl, method 7 bank 2, Loop 1 mL. Two fractions were collected: one with the product and one with the residue. Relative absorbance PNA(Flu) (ε_{500 nm}=73000 M⁻¹) and GAM (ε_{278 nm}=213000 M⁻¹) (correction factor for PNA at 278 nm is due to the specific PNA and is calculated: 278/500 nm) was used to calculate the average conjugation ratio of PNA, GAM and DexVS70. The ratio of PNA to DexVS70 was 5.3 and the ratio of GaM to DexVS70 was 0.8.

### Example 13

### Exemplary Embodiments of PNA1-DexVS-PNA2 Conjugates

TABLE 2

| | | | | PNA1 | | | PNA2 | |
|---|---|---|---|---|---|---|---|---|
| Conjugate | | | PNA1 | to | | PNA2 | to | |
| designation | ratio | PNA1 | nmol | DexVS | PNA2 | nmol | DexVS | DexVS |
| Conj. CA | 1:9 | Seq. AA | 12.5 | 1.02 | Seq. AD | 100 | 8.2 | DexVS70 |
| Conj. CB | 1:6 | Seq. AC | 40 | 1.5 | Seq. AB | 200 | 7.4 | DexVS70 |
| Conj. CC | 1:16 | Seq. AC | 13.3 | 0.84 | Seq. AB | 200 | 12.7 | DexVS150 |
| Conj. CD | 1:6 | Seq. AC | 40 | 2.3 | Seq. AB | 200 | 11.5 | DexVS150 |

All conjugates were made by standard conjugation procedures of Example 17.

### Example 14

### Synthesis of Anti-Human-BCL2-DexVS70-PNA

Dextran (molecular weight 70 kDa) was activated with divinylsulphone to a degree of 92 reactive groups/dextran polymer, and is designated DexVS70. The antibody Anti-Human-BCL2 is designated AHB.

| | |
|---|---|
| 105 μL DexVS70 | 7.5 nmol |
| 800 μL AHB conc. (2.9 g/L) | 15.1 nmol |
| 25 μL 4 M NaCl | |
| 32 μL 0.8 M NaHCO₃ (pH = 9.5) | |

The above four compounds were mixed and placed in a water bath at 30° C. for 65 minutes. From this mixture, 875 μL was taken out and added to the indicated number of nmol of PNA in the table below; before the addition the PNA had been dissolved in the μL of H₂O indicated in the table below. Then the number of μLs of 0.8 M NaHCO₃ (pH 9.5) was added according to the table below. The resulting mixture was placed in a water bath at 30° C. for 70 minutes. Quenching was performed by adding 6 mg of solid cysteine (0.05 M) to the mixture and letting it stand for 30 minutes at 30° C.

Purification of the product on FPLC: column SUPERDEX®—200, buffer 10 mM Hepes 100 mM NaCl, method 7 bank 2, Loop 1 mL. Two fractions were collected: one with the product and one with the residue. Relative absorbance PNA(Flu) (ε_{500 nm}=73000 M⁻¹) and AHB (ε_{278 nm}=213000 M⁻¹) (correction factor for PNA at 278 nm is due to the specific PNA and is calculated: 278/500 nm) was used to calculate the average conjugation ratio of PNA, AHB and DexVS70.

Conjugates with different ratios PNA are shown in the following table.

TABLE 3

| | nmol of | μL of | μL of 0.8 M | | |
|---|---|---|---|---|---|
| Conjugate | PNA | H₂O | NaHCO₃ | PNA to | AHB to |
| designation | added | added | (pH 9.5) added | DexVS70 | DexVS70 |
| Conj. DA | 100 | 75 | 25 | 9.5 | 1.6 |
| Conj. DB | 33 | 30 | 10 | 2.9 | 1.2 |
| Conj. DC | 67 | 60 | 20 | 5.6 | 1.1 |

### Example 15

### Solid Phase Synthesis and Purification of Lys(FIu)-L₃₀-chr 17:14-L₃₀-Lys(Flu)-L₉₀-Lys(Flu)-L₉₀-Lys(Flu)

All Standard procedures are described in Example 18.

1. An MBHA-resin was loaded with Boc-L₃₀-Lys(Fmoc)-L₉₀-Lys(Fmoc)-L₉₀-Lys(Fmoc) using a standard loading procedure to a loading of 0.084 mmol/g.

2. To this resin, Boc-Lys(Fmoc)-L₃₀-AAC-GGG-ATA-ACT-GCA-CCT- was coupled using the peptide synthesizer machine following standard PNA solid phase chemistry. Fmoc protection groups were removed and the amino groups were labeled with fluorescein. After cleaving and precipitation the PNA was dissolved in TFA. The precipitate was washed with ether. The precipitate was dissolved in 200 μL NMP To this solution 6 mg Fmoc-Osu was added and dissolved. Next, DIPEA (9 μL) was added and the reaction was followed using MALDI-TOF mass spectrometry. After 30 minutes the reaction was finished and the PNA was precipitated and washed with ether.

HPLC after dissolving the PNA in 30% CH₃CN and 10% TFA/H₂O gave three pure fractions. The fractions were pooled and lyophilized. The lyophilized PNA was then dissolved in 192 μL NMP. Piperidine (4 μL) and 4 μL DBU was added to this solution which set for 30 minutes. Analysis by MALDI-TOF mass spectrometry gave a molecular weight of 10777.

The precipitate was washed with ether and was then dissolved in 100 μL TFA. The precipitate was washed with ether and then dried using N₂ gas.

### Example 16

### Standard Synthesis of HRP-DexVS70-PNA Conjugate

Dextran (molecular weight 70 kDa) is activated with divinylsulfone to a degree of 92 reactive groups/dextran polymer.

| | |
|---|---|
| 192 μL DexVS70 | 13.7 nmol |
| 255 μL horse radish peroxidase (HRP) | 602 nmol |
| 15 μL 4 M NaCl | |
| 19 μL 0.8 M NaHCO₃ pH = 9.5 | |
| 119 μL H₂O | |

The above five components are mixed together placed in a water bath at 30° C. for 16 hours. Five hundred microliters of this mixture are added to 50 nmol PNA, which is previously dissolved in 40 μL H₂O. Then, 10 μL of 0.8 M NaHCO₃ (pH 9.5) is added. The mixture is then placed in a water bath at 30° C. for 2 hours. Quenching is performed by adding 55 μL of 110 mM cysteine and letting the resultant mixture set for 30 minutes at 30° C.

Purification of the product is performed by FPLC: column SUPERDEX®—200, buffer 10 mM Hepes 100 mM NaCl, method 7 bank 2, Loop 1 mL.

Two fractions are collected: one with the product and one with the residue. Relative absorbance HRP (ε_{404 nm}32 83000 M⁻¹, ε_{500 nm}=9630 M⁻¹) and PNA(Flu) (ε_{500 nm}=73000 M⁻¹) is used to calculate the average conjugation ratio of HRP, PNA and DexVS70.

### Example 17

### Standard Synthesis of GAM-DexVS70-PNA Conjugate

Dextran (molecular weight 70 kDa) is activated with divinylsulfone to a degree of 92 reactive groups/dextran polymer (DexVS70).

| | |
|---|---|
| 105.0 μL DexVS70 | 7.5 nmol |
| 57.0 μL Goat anti mouse Imuno globuline (GAM) | 15 nmol |
| 8.9 μL 4 M NaCl | |
| 10.6 μL 0.8 M NaHCO₃ (pH = 9.5) | |
| 144.5 μL H₂O | |

The above five components are mixed and placed in a water bath at 30° C. for 40 minutes. Two hundred and ninety μL is taken out of the mixture and added to 50 nmol of PNA, which is previously dissolved in 40 μL of H₂O. Then, 10 μL of 0.8 M NaHCO₃ (pH 9.5) is added and the mixture placed in a water bath at 30° C. for 1 hour. Quenching is performed by adding 34 μL of 500 mM cysteine and letting the resultant mixture set for 30 minutes at 30° C.

Purification of the product on FPLC: column SUPERDEX®—200, buffer 10 mM Hepes 100 mM NaCl, method 7 bank 2, Loop 1 mL. Two fractions are collected: one with the product and one with the residue. Relative absorbance PNA(Flu) (ε_{500 nm}=73000 M⁻¹) and GAM (ε_{278 nm}=213000 M⁻¹) (correction factor for PNA at 278 nm is due to the specific PNA and is calculated: 278/500 nm) was used to calculate the average conjugation ratio of PNA, GAM and DexVS70.

### Example 18

### Standard Synthesis of PNA1-DexVS70-PNA2

Dextran (molecular weight 70 kDa) is activated with divinylsulfone to a degree of 92 reactive groups/dextran polymer. PNA1 (100 nmol) is dissolved in 140 μL of DexVS70 (10 nmol). To this mixture 12.5 μL of PNA2 (12.5 nmol) dissolved in H₂O is added, and then 30 μL of NaHCO₃ (pH 9.5) is added and the solution mixed. The resultant mixture is placed in a water bath at 30° C. for 35 minutes. Quenching was performed by adding 18.3 μL of 500 mM cysteine in Hepes and letting this mixture set for 30 minutes at 30° C.

Purification of the product on FPLC: column SUPERDEX®—200, buffer 10 mM Hepes 100 mM NaCl, method 7 bank 2, Loop 1 mL. Two fractions are collected: one with the product and one with the residue. Relative absorbance PNA(Flu) (ε_{500 nm}=73000 M⁻¹) and the proportion between the two PNA's is used to calculate the average conjugation ratio of PNA, PNA and DexVS70.

### Example 19

### Synthesis of the Boc-PNA-I(O-Bz)-Monomer

6-Benzyloxypurine. Sodiumhydride (60% Dispersion in mineral oil; 3.23 g; 80 mmol) was slowly added to benzyl alcohol (30 ml; 34.7 mmol). After the addition of more benzyl alcohol (10 ml) and 6-chloropurine (5.36 g;). The reaction mixture was heated to 100° C. for 4 hours. When the reaction mixture has reached room temperature, water (1 ml) was slowly added. 6-Benzyloxypurine was precipitated by the addition of acetic acid (4.6 ml) and diethylether (550 ml). The precipitate was separated by filtration (11.72 g). Re-crystallization from ether gave (4.78 g; 65.4%). Melting point was 175-177° C. (litt. 170-172° C)[Ramazaeva N., 1989 #473] 1H-NMR (DMSO-d6): 8.53 (1H, s); 8.39 (1H, s); 7.54-7.35 (5H, m); 5.62 (2H,s).

Methyl(6-(Benzyloxy)purin-9-yl)acetate. 6-Benzyloxypurine (4.18 g; 18.5 mmol) was added to a suspension of potassium carbonate (3.1 g; 22.4 mmol) in DMF (100 ml). After 15 min., bromoacetic acid methyl ester (1.93 ml; 20.4 mmol) was added. The reaction was monitored by TLC in butanol:acetic acid:water 4:1:1. Upon completion, the reaction mixture was partitioned between water (600 ml) and ethyl acetate (600 ml). The organic phase was dried over magnesium sulfate and evaporated to a volume of ˜10 ml and precipitated with pet. ether. The two products were separated by column chromatography using ethyl acetate as the solvent. The products were precipitated in pet. ether. Yield: 2.36 g (43%). Melting point: 111.5-115° C. UV λmax=250 nm (9-alkylated); λmax=260 nm (7-alkylated). 1H-NMR (DMSO-d6): 8.60 (1H, s); 8.43 (1H,s); 7.6-7.35 (5H, m); 5.69 (2H, s); 5.26 (2H, s); 3.75 (3H, s).

(6-(Benzyloxy)purin-9-yl)acetic acid. Methyl(6-(Benzyloxy)purin-9-yl)acetate (2.10 g; 7.0 mmol) was dissolved in methanol (70 ml) and 0.1 M NaOH (85 ml) is added. After 15 min. the pH of the reaction mixture was lowered by addition of 0.1 M HCl (˜80 ml) to pH 3. The precipitate was separated from the mixture by filtration and washed with water and ether. Yield: 1.80 g (90.2%). 1H-NMR (DMSO-d6): 8.55 (1H,s); 8.37 (1H,s); 7.55-7.30 (5H, m); 5.64 (2H, s); 5.09 (2H, s).

N-((6-(Benzyloxy)purin-9-yl)acetyl)-N-(2-Boc-aminoethyl)glycine. Ethyl N-(2-Boc-aminoethyl)glycinate (0.285 g; 1.15 mmol), (6-(benzyloxy)purin-9-yl)acetic acid (0.284 g; 1.0 mmol) and 3-hydroxy-1,2,3benzotriazin-4(3H)-one (0.180; 1.1 mmol) was dissolved in dichlormethane/dimethylformamide 1:1 (10 ml). After addition of dicycloehexylcarbodiimide (0.248 g; 1.2 mmol) the reaction was left over night. The precipitate was removed by filtration. The organic phase was extracted twice with saturated sodium bicarbonate, dried with magnesium sulfate and evaporated to a oil. Column purification on silica using dichloromethane with 0-5% methanol as elutant yields the monomer ester which was dissolved in methanol (10 ml). Then, 0.1 M NaOH (12 ml) was added. After 30 min the reaction was filtered and pH adjusted with saturated KHSO4/water (1:3) to 2.7. The water phase was extracted twice with ethyl acetate (2×100 ml). The combined organic phases were dried over magnesium sulfate and evaporated to a volume of 10 ml. Precipitation with pet. ether yielded the monomer (0.15 g; 31%). 1H-NMR (DMSO-d6): 8.51 (1H, s); 8.23 (1H, s); 7.6-7.3 (5H, m); 5.64 (2H, s); 5.31 (ma.) +5.13 (mi.) (2H, s); 4.23 (mi.) +3.98 (ma.) (2H, s); 3.55-3.00 (4H, m); 1.36 (9H, s).

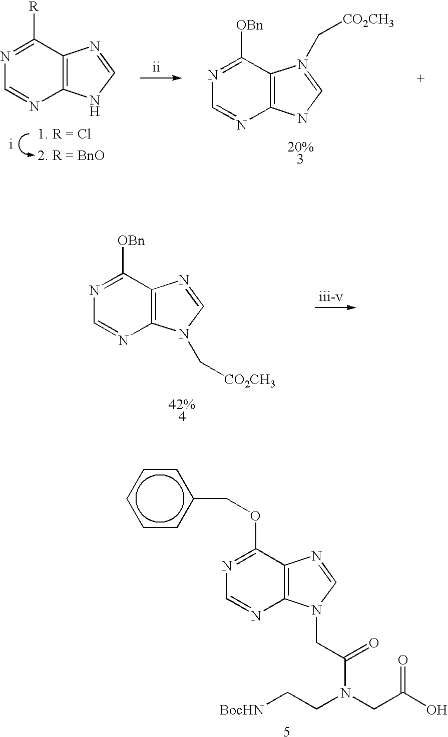

The synthesis of the hypoxanthine PNA monomer. (i) BnOH, NaH (ii) K2CO3, BrCH2CO2CH3 (iii) OH— (iv) DCC, Dhbt-OH, Boc-aeg-OEt (v) OH—

The Boc-PNA-Diaminopurine-(N6-Z)-monomer was prepared according to Gerald Haaima, Henrik F. Hansen, Leif Christensen, Otto Dahl and Peter E. Nielsen; Nucleic Acids Research, 1997, Vol 25, Issue 22 4639-4643.

The Boc-PNA-2-Thiouracil-(S-4-MeOBz)-monomer was prepared according to Jesper Lohse, Otto Dahl and Peter E. Nielsen; Proceedings of the National Academy of Science of the United States of America, 1999, Vol 96, Issue 21, 11804-11808.

The Boc-PNA-Adenine-(Z)-monomer was from PE Biosystems catalog GEN063011.

The Boc-PNA-Cytosine-(Z)-monomer was from PE Biosystems cat. GEN063013.

The Boc-PNA-Guanine-(Z)-monomer was from PE Biosystems cat. GEN063012.

The Boc-PNA-Thymine-monomer was from PE Biosystems cat. GEN063010.

IsoAdenine (2-aminopurine) may be prepared as a PNA-monomer by 9-N alkylation with methylbromoacetate, protection of the amino group with benzylchloroformate, hydrolysis of the methyl ester, carbodiimide mediate coupling to methyl-(2-Boc-aminoethyl)-glycinate, and finally hydrolysis of the methyl ester.

4-thiouracil may be prepared as a PNA-monomer by S-protection with 4-methoxy-benzylchloride, 1-N alkylation with methylbromoacetate, hydrolysis of the methyl ester, carbodiimide mediate coupling to methyl-(2-Boc-aminoethyl)-glycinate, and finally hydrolysis of the methyl ester.

Thiocytosine may be prepared as a PNA monomer by treating the Boc-PNA-cytosine(Z)-monomer methyl ester with Lawessons reagent, followed by hydrolysis of the methyl ester.

A number of halogenated bases are commercially available, and may be converted to PNA monomers analogously to the non-halogenated bases. These include the guanine analog 8-bromo-guanine, the adenine analogs 8-bromo-adenine and 2-fluoro-adenine, the isoadenine analog 2-amino-6-chloro-purine, the 4-thiouracil analog 5-fluoro-4-thio-uracil, and the 2-thiouracil analog 5-chloro-2-thiouracil.

Boc-PNA-Uracil monomers were first described in “Uracil og 5-bromouracil I PNA,” a bachelor project by Kristine Kils\dot{a} Jensen, Kφbenhavns Universitet 1992.

### Example 20

### Miscellaneous Standard Procedures

a. Loading of resins. P-methyl-BHA-resin (3 g) is loaded with Boc-Lys(Fmoc)-OH 15 mmol/g resin. The lysine is dissolved in NMP and activated with 0.95 equivalents (eq.) HATU and 2 eq. DIPEA. After loading the resin, it is capped by adding a solution of (Ac)₂O/NMP/pyridine (at a ratio of 1/2/2) and letting it set for at least 1 hour or until Kaiser test was negative. After washing with DCM, the resin is dried in a dessicator. Quantitative Kaiser test typically gives a loading of 0.084 mmol/g.

b. Amino Acid Couplings. The Boc protection group is removed from the resin with TFA/m-cresol (at a ratio of 95/5) 2×5 min. The resin is then washed with DCM, pyridine and DMF before coupling with the amino acid, which is dissolved in NMP in a concentration between 0.2 and 0.4 M and activated with 0.95 eq. of HATU and 2 eq of DIPEA for 2 minutes. The coupling is complete when the Kaiser test is negative. Capping occurring by exposing the resin for 3 minutes to (Ac)₂O/pyridine/NMP (at a ratio of 1/2/2). The resin is then washed with DMF and DCM

c. Boc-L₃₀₀-Lys(Fmoc)-resin. To the loaded Boc-Lys(Fmoc)-resin, L₃₀-Linker in a concentration of 0.26 M was coupled using standard amino acid coupling procedure. This was done 10 times giving Boc-L₃₀₀-Lys(Fmoc)-resin.

d. PNA solid phase. On a peptide synthesizer (ABI 433A, Applied Biosystems) PNA monomers are coupled to the resin using standard procedures for amino acid coupling and standard PNA chemistry. Then the resin is handled in a glass vial to remove protections groups and to label with either other amino acids or flourophores.

Removal of the indicated protection groups is achieved with the following conditions:

Boc: TFA/m-cresol (at a ratio of 95/5) 2×5 min.

Fmoc: 20% piperidine in DMF 2×5 min.

Dde: 3% hydrazine in DMF 2×5 min.

When the synthesis is finished, the PNA is cleaved from the resin with TFA/TFMSA/m-cresol/thioanisol (at a ratio of 6/2/1/1). The PNA is then precipitated with ether and purified on HPLC. MALDI-TOF mass spectrometry is used to determine the molecular weight of the product.

e. Labeling with fluorescein. 5(6)-carboxy fluorescein is dissolved in NMP to a concentration of 0.2 M. Activation is performed with 0.9 eq. HATU and 1 eq. DIPEA for 2 min before coupling for at least 2×20 min or until the Kaiser test is negative.

### Example 21

### PNA with Positive and Negative Loadings

In order to make better conjugations at one time we tried to give the PNA a loading. Both PNA's were made by PNA standard procedures (See Example 20).

1. FIu-L₃₀-Glu-TCA-AGG-TAC-A-Glu-L₃₀₀-Lys(Cys)

Glu=glutamate has negative loadings and for the easiness the PNA is designated −A4−

2. Flu-L₃₀-Lys(Me)₂-TGT-ACC-TTG-A-Lys(Me)₂-L₃₃₀-Lys(cys)

Lys(Me)₂=Boc-Lys(Me)₂-OH has positive loadings and the PNA is designated +T+

TABLE 4

| | | | | | | GaM/ | PNA/ |
|---|---|---|---|---|---|---|---|
| name | number | HRP | GaM | equiv. | HRP/Dex | Dex | Dex |
| −A4− | D13041 | D13050 | | 9 | 12.3 | | 0.13 |
| −A4− | D13041 | | D13060 | 7 | | 0.94 | 0.66 |
| +T4+ | D13042 | D13058 | | 9 | 13.5 | | 0.19 |
| +T4+ | D13042 | | D13056 | 7 | | 1.42 | 0.45 |

As it is shown in the scheme, PNAs with loading are not good at coupling.

### Example 22

### Target Detection: Procedures used in the Examples Below

### 1. Fixation of Biological Samples

Tonsil tissue samples were fixed in neutral buffered formalin, NBF (10 mM NaH₂PO₄/Na₂HPO₄, pH 7.0), 145 mM NaCl, and 4% formaldehyde (all obtained from Merck, Whitehouse Station, N.J.). The samples were incubated overnight in a ventilated laboratory hood at room temperature.

### 2. Sample Dehydration and Paraffin Embedding

The tissue samples were placed in a marked plastic histocapsule (Sakura, Japan). Dehydration was performed by sequential incubation in 70% ethanol twice for 45 min, 96% ethanol twice for 45 min, 99% ethanol twice for 45 min, and xylene twice for 45 min. The samples were subsequently transferred to melted paraffin (melting point 56-58° C.) (Merck, Whitehouse Station, N.J.) and incubated overnight (12-16 hours) at 60° C. The paraffin-infiltrated samples were transferred to fresh warm paraffin and incubated for an additional 60 min prior to paraffin embedding in a cast Sekura, Japan). The samples were cooled to form the final paraffin blocks. The marked paraffin blocks containing the embedded tissue samples were stored at room temperature in the dark.

### 3. Cutting, Mounting and Deparaffination of Embedded Samples

The paraffin blocks were cut and optionally also mounted in a microtome (0355 model RM2065, Feather S35 knives, set at 5.0 micrometer; Leica, Bannockburn, Ill.). The first few millimeters were cut and discarded. Paraffin sections 4-6 micrometers thick were then cut and collected at room temperature. The sections were gently stretched on a 45-60° C. hot water bath before being mounted onto marked microscope glass slides (SUPERFROST® Plus; Fisher, Medford, Mass.), two tissue sections per slide. The slides were then dried and baked in an oven at 60° C. The slides were deparaffinated by incubating twice in xylene for 5 min±2 min twice, then in 96% ethanol for 2 min±30 sec, then twice in 70% ethanol for 2 min±30 sec, and then once in Tris-buffered saline with TWEEN® (called herein TBST) for 5 min. TBST comprises 50 mM Tris adjusted to pH 7.6 with HCl; 150 mM NaCl; 0.05% TWEEN®20. The slides were deparaffinated by subsequently incubation in xylene twice for 5 min±2 min, 96% ethanol twice for 2 min±30 sec and 70% ethanol twice for 2 min±30 sec. The slides were immersed in deionized water and left for 1 to 5 min.

### 4. Endogenous Peroxidase Blocking

Samples were incubated with a 3% hydrogen peroxide solution for 5 min. to quench endogenous peroxidase activity, followed by washing in deionized water for 1 to 5 min.

### 5. Antigen Retrieval by Microwave Oven

Antigens in the sample were retrieved by immersing the slides in a container containing Antigen Retrieval Solution, pH 6.0 (DakoCytomation code No. K5204 Vial 7 or optional code No. K5205 Vial 7). The container was closed with a perforated lid and placed in the middle of a microwave oven and left boiling for 10 min. The container was removed from the oven and allowed to cool at room temperature for 20 min. The samples were rinsed in deionized water.

### 6. Antigen Retrieval by Water Bath Incubation

Antigens in the sample were retrieved by immersing the slides in a beaker containing Antigen Retrieval Solution, pH 6.0 (DakoCytomation code No. K5204 Vial 7 or optional code No. K5205 Vial 7). The samples were incubated for 40 min in a water bath at 95-100° C. The beaker was removed from the water bath and allowed to cool at room temperature for 20 min. The samples were rinsed in deionized water.

### 7. Water-Repellent Barrier to Liquids by DakoCytomation Pen

To ensure good coverage of reagent on the tissue sample, the area on the slide with tissue was encircled with a silicone rubber barrier using DakoCytomation Pen (DakoCytomation code No. 2002). The slides were transferred to a rack and placed in a beaker containing Tris-buffered saline with TWEEN® (called herein TBST) and left for 5 min. TBST comprises 50 mM Tris adjusted to pH 7.6 with HCl; 150 mM NaCl; 0.05% TWEEN®20.

### 8. Application of a Primary Antibody

Monoclonal Mouse anti-Human Cytokeratin (DakoCytomation code No. M3515) diluted 1:900 in ChemMate™ Antibody Diluent (DakoCytomation code No.S2022) was applied on the tissue samples and incubated for 30 min in a humid chamber at ambient temperature. The slides were individually rinsed and then washed in TBST for 5 min.

### 9. Application of Three Primary Antibodies

Monoclonal Mouse Anti-Human Cytokeratin (DakoCytomation code No. M3515) diluted 1:300, 1:900 and 1:1600; monoclonal Mouse Anti-Human CD20cy (DakoCytomation code No. M0755) diluted 1:2000, 1:8000 and 1:14000; and monoclonal Mouse Anti-Human Ki-67 Antigen (DakoCytomation code No. M7240) diluted 1:400, 1:1200 and 1:2400 were used. The antibodies were diluted in ChemMate™ Antibody Diluent (DakoCytomation code No. S2022), applied on the tissue samples, and incubated for 30 min in a humid chamber at ambient temperature. The slides were individually rinsed and washed in TBST for 5 min.

### 10. Application of an Antibody/Dextran/PNA1 Coniuqate Recognition Unit

Antibody/Dextran/PNA1 conjugate recognition unit is also called “PNA1 conjugate” in the examples that follow. The PNA1 conjugate comprises 70,000 molecular weight dextran. Table 5 summarizes PNA1 conjugates based on a secondary antibody: goat anti-mouse Ig, called herein GAM (DakoCytomation code No. Z0420). Table 6 summarizes PNA1 conjugates based on a primary antibody: mouse anti-human BCL2 oncoprotein, such as Clone 124 (DakoCytomation code No. M0887). The primary antibody was protein A-purified prior to conjugation. The conjugates were diluted in BBA (50 mM Tris adjusted to pH 7.6 with HCl; 150 mM NaCl; 2% BSA; 0.02% bronidox; 2.44 mM 4-aminoantipyrin) and were applied on the tissue sample in a range of dilutions, then incubated for 30 min in a humid chamber at ambient temperature. The slides were individually rinsed and washed in TBST for 5 min.

TABLE 5
PNA1 conjugates useful in indirect recognition
of targets: GAM/Dextran/PNA1

| Conjugate | | μM | GAM/ | PNA1/ |
|---|---|---|---|---|
| No. | Sequence | Dex | Dex | Dex |
| D14120 | AGA CPT TPG DPT | 1.25 | 1.1 | 4.3 |
| D14102 | GTP TAA TTP PAG | 1.02 | 1.0 | 9.1 |
| D14096 | GTP TAD TTP PAG | 1.15 | 1.4 | 4.2 |
| D14083 | U_{S}GU_{S} DPP TTG D | 0.87 | 0.8 | 5.3 |
| D13171 | U_{S}GU_{S} DPP TTG D | 1.21 | 1.0 | 7.5 |
| D13161 | TTG APP TTA G | 2.11 | 1.1 | 6.0 |
| D13150 | TGT APP TTGA | 2.20 | 1.1 | 4.2 |
| D13102 | TGT ACC TTGA | 2.53 | 1.1 | 2.5 |
| D12102 | TGT ACC TTGA | 2.50 | 1.3 | 4.5 |

TABLE 6
PNA1 conjugates for direct recognition of
targets: anti-BCL2/Dextran/PNA1

| Conjugate | | μM | Ab/ | PNA1/ |
|---|---|---|---|---|
| No. | Sequence | Dex | Dex | Dex |
| D14128 | U_{S}GU_{S} DPP TTG D | 0.8 | 1.1 | 5.6 |
| D14126 | U_{S}GU_{S} DPP TTG D | 1.0 | 1.2 | 2.9 |
| D14122 | U_{S}GU_{S} DPP TTG D | 1.1 | 1.6 | 9.5 |

In the above tables, the letters A, C, G, U, and T, stand for the natural bases adenine, cytosine, guanine, uracil, and thymine. P stands for pyrimidinone, D for 2,6-diaminopurine, and U_{S} for 2-thiouracil.

11. Fixation of PNA1-Conjugate with 1% Glutardialdehyde

The samples were washed in deionized water for 30 sec. Then, 1% glutardialdehyde (Merck Art. No. 820603), called herein GA, diluted in 22 mM calcium phosphate buffer, pH 7.2, was applied, and the samples were incubated for 10 min in a humid chamber at ambient temperature. The samples were washed in deionized water for 30 sec and in TBST for 5 min.

### 12. Application of a PNA¹-PNA²/Dextran Conjugate Adaptor Unit

PNA¹-PNA²/Dextran conjugate is also called “PNA¹-PNA²” in the following examples. Table 7 summarizes the compositions of PNA¹-PNA² conjugates. PNA¹ is complementary to the PNA1 conjugate, and PNA² is complementary to the PNA2 conjugates D14079 and D13155 described in step 13 below. The sequence of PNA¹ is CU_{S}G_{S} G_{S}DD TU_{S}D G_{S}DC and the sequence of PNA² is U_{S}GU_{S} DPP TTG D, in which U_{S} stands for 2-thio-uracil, G_{S} stands for 2-amino-6-thioxopurine, D stands for diaminopurine, and P stands for pyrimidinone. The conjugates, diluted in BBA, were applied to the tissue samples in a range of dilutions, and the samples were then incubated for 30 min in a humid chamber at ambient temperature. The samples were individually rinsed and washed in TBST for 5 min. When testing a PNA¹-PNA²conjugate, fixed concentrations of 0.08 μM PNA1 and 0.05 μM PNA2 were used.

TABLE 7
PNA¹-PNA²/Dextran conjugates

| | Molecular | | | |
|---|---|---|---|---|
| Conjugate | weight | | | |
| No. | of dextran | PNA¹/dex | PNA²/dex | μM PNA¹ |
| D14119 | 150.000 | 2.3 | 11.5 | 4.2 |
| D14106 | 150.000 | 0.8 | 12.7 | 1.3 |
| D14104 | 70.000 | 1.5 | 7.5 | 3.9 |

### 13. Application of Horse Radish Peroxidase/Dextran/PNA2 Conjugate Detection Unit

Horse Radish Peroxidase (HRP)/Dextran/PNA2 conjugates are also called “PNA2 conjugate” in the examples that follow, and are listed in table 8. The PNA2 conjugates comprise 70.000 Da molecular weight dextran. The conjugates diluted in BBA were applied to the tissue samples in a range of dilutions, and samples were incubated for 30 min in a humid chamber at ambient temperature. The samples were individually rinsed and washed twice in TBST for 5 min.

TABLE 8
PNA2 conjugates: HRP/Dextran/PNA2

| Conjugate | | μM | HRP/ | PNA2/ |
|---|---|---|---|---|
| No. | Sequence | PNA | Dex | Dex |
| D14133 | TCD DII TAC A | 1.6 | 14.0 | 1.0 |
| D14114 | DG_{S}T CG_{S}D DG_{S}G U_{S}CU_{S} | 3.9 | 11.4 | 2.1 |
| D14110 | DGT CG_{S}D DG_{S}G U_{S}CU_{S} | 3.0 | 12.6 | 1.6 |
| D14089 | CU_{S}G_{S} G_{S}DD TU_{S}D G_{S}DC | 2.1 | 14.1 | 1.5 |
| D14086 | U_{S}CG_{S} G_{S}DD TU_{S}D GDC | 1.9 | 11.0 | 1.0 |
| D14079 | TCD DG_{S}G_{S} TAC A | 1.9 | 12.2 | 1.2 |
| D13159 | CTA AG_{S}G_{S} TCA A | 1.9 | 12.9 | 1.3 |
| D13155 | TCD DG_{S}G_{S} TAC A | 2.4 | 12.7 | 1.6 |
| D13148 | TCA AG_{S}G_{S} TAC A | 1.9 | 11.6 | 0.8 |
| D13122 | CTA AGG TCA A | 3.2 | 13.0 | 2.1 |
| D13108 | GTG TGT GT | 4.3 | 12.0 | 2.3 |
| D13106 | TCA AGG TAC A | 2.6 | 12.4 | 1.3 |
| D12120 | TCD DGG TAC A | 1.0 | 18.3 | 0.6 |
| D12094 | TCA AGG TAC A | 3.0 | 14.6 | 0.9 |

In table 8, in addition to the nucleobase letter schemes provided for Tables 5-7, I stands for inosine.

### 14. Application of Diaminobenzidine Chromogenic Substrate Solution

The diaminobenzidine chromogenic substrate solution, DAB+ (DakoCytomation code No. K3468) was applied on the tissue samples, and the samples were incubated for 10 min in a humid chamber at ambient temperature. The samples were washed with deionized water for 5 min.

15. Counterstaining with Hematoxylin

The tissue samples were immersed in Mayers Hematoxylin (Bie & Berntsen Code No. LAB00254) for 3 min, rinsed in tap water for 5 min, and finally rinsed with deionized water.

### 16. Cover Slipping

Cover slips were applied to the tissue samples using the aqueous mounting media, Faramount (DakoCytomation code No. S3025).

### 17. Evaluation of the Performance

The tissue staining was examined in a bright field microscope at 10×, 20× or 40× magnification. Both the specific and the non-specific staining intensity were described with a score-system using the range 0 to 3+ with 0.5+ score interval. ChemMate™ EnVision™ Detection kit Rabbit/Mouse (DakoCytomation code No. K5007 bottle A) was used as a reference, and was included in all experiments for testing in parallel with the PNA conjugates. K5007 was used according to manufacturer's instructions. The antibodies were used in the following dilutions: M3515 at 1:900, M0755 at 1:8000, and M7240 at 1:1200. The staining intensity of the K5007 reference using the primary antibody M3515 diluted 1:900 was set to 230 in order to compare and assess the staining result of the PNA conjugate tested. If the reference deviated more than ±0.5, the test was repeated.

In the examples, the various visualization system combinations of the invention were tested on routine tissue samples. The staining performance was compared with a reference visualization system, using EnVision™ and a very dilute antibody from DakoCytomation. The practical dynamic range of quantitative IHC may be narrow, and e.g. strongly stained (+3) tissues are not easy to compare with respect to intensity. Therefore, on purpose, the staining intensity of the reference system was adjusted to be approximately +2. This was done in order to better monitor and compare differences in staining intensity with the system of the invention.

### Example 23

### Protocol for Fast Evaluation of Non-Specific Binding of PNA2 Conjugates

The protocol allowed for a quick test of PNA2 conjugates for non-specific staining. Tonsil tissues were taken through the steps 1-5, 7, 13-14, 15 (in which the slides were immersed in a bath of Hematoxylin Mayer for 1 min.), and 16-17, above. The conjugates to be tested were diluted to the final concentrations 0.05 μM and 0.2 μM. As references, two PNA2 conjugates were used in the final concentration 0.05 μM. The first reference, for example, PNA2, D13108, was known to give non-specific nuclear staining, and so was used as a positive control. The second reference, for example, PNA2, D13155, was known not to give any non-specific nuclear staining, and was used as a negative control. In general, 250 μL of each reagent was applied unless otherwise specified.

Protocol for Test of a PNA Pair with One Antibody.

Tonsil tissues were taken through the steps 1-4, 6-8, 10, 11, and 13-17 above. Step 11 was left out for the tonsils not fixed with 1% GA. In general, 250 μL of each reagent was applied unless otherwise specified.

### Protocol for Test of 3-Layer PNA Conjugates

Tonsil tissues were taken through the steps 1-4, 6-8, and 10-17 above. Step 11 was left out for the tonsils not fixed with 1% GA. In general, 250 μL of each reagent was applied unless otherwise specified.

Protocol for Test of a PNA Pair with 3 Antibodies

Tonsil tissues were taken through the steps 1-4, 6, 7, 9-11, and 13-17 above. Step 11 was left out for the tonsils not fixed with 1% GA. A further negative control, mouse IgG1 (DakoCytomation code No. X0931) diluted 1:300 in S2022 was included the protocol for the PNA conjugates. In general, 250 μL of each reagent was applied unless otherwise specified.

### Example 24

### Testing and Selection of PNA Pairs

Conjugates comprising example PNA segments were tested for their ability to specifically hybridize according to the invention. Tonsil tissues were taken through the steps 1-4, 6-8, 10 and 13-17 above. K5007 was included as a reference to secure the level of the staining. The concentration of the conjugates was 0.08 μM for PNA1 and 0.05 μM for PNA2.

The results listed in Tables 9 and 10 show the staining intensities for a representative number of PNA pairs tested. The PNA pairs did not demonstrate any non-specific binding. The specific staining, in general, was directly proportional to the number of hydrogen bonds involved in the base-pairing. It was important that each PNA did not interact with itself. Substitution of T (thymine) with U_{S} (2-thiouracil) in some PNAs could prevent such intra-PNA interactions. The unspecific staining intensity was increased by substituting an A with a D. On the other hand, we also observed that replacement of one G (guanine) with G_{S} (2-amino-6-thioxopurine) in the same PNA could circumvent the unspecific binding introduced by D. For instance, the staining of the PNA1 conjugate D14102 was improved by substituting the D in D14096 with an A in D14102. As is apparent from Table 9, this small change resulted in an increase of the specific staining score by 1+.

TABLE 9

| | | Specific | Non-specific |
|---|---|---|---|
| PNA1 | PNA2 | staining intensity | staining intensity |
| D13161 | D13159 | 2 | 0 |
| D14083 | D14079 | 2.5 | 0 |
| D14096 | D14089 | 1.5 | 0 |
| D14102 | D14089 | 2.5 | 0 |
| D14120 | D14114 | 1.5 | 0 |

### Example 25

### The Effect of Base Substitution on PNA-Specific Binding Intensities

The PNA pair D13102-D13106 was used as a starting point for further investigation of introducing base substitutions in either PNA1 or PNA2 conjugates. Tonsil tissues were taken through the steps 1-4, 6-8, 10 and 13-17. Each of the three different PNA1 conjugates was tested with each of the three different PNA2 conjugates. The concentration of the conjugates used was 0.08 μM for PNA1 and 0.05 μM for PNA2.

TABLE 10

| | PNA2: | | |
|---|---|---|---|
| | D13106 | D13148 | D13155 |
| | TCA AGG | TCA AGₛGₛ | TCD DGₛGₛ |
| PNA1: | TAC A | TAC A | TAC A |
| D13102 | | | |
| TGT ACC | 2.5 | 0 | 2 |
| TTG A | | | |
| D13150 | | | |
| TGT APP | 2.5 | 0.5 | 3 |
| TTG A | | | |
| D13171 | | | |
| UₛGUₛ DPP | 3 | 2.5 | 3 |
| TTG D | | | |

Table 10 shows the effect of base substitutions on the specific binding between paired PNA variants. No non-specific binding was observed. D13102 tested with D13106 gave a specific staining of 2.5+. Replacement of 2 G's with 2 G_{S}'s (D13148) resulted in the abolishment of specific staining, but by introducing 2 D's instead of 2 A's (D13155) achieved a specific staining of 2+. When the 2 C's in D13102 were replaced with 2 P's (D13150) and tested with D13106, the specific staining was unchanged at 2.5+, despite the lower number of hydrogen bonds as compared to the PNA-pair D13102-D13106. Test of D13150 with D13148 resulted in a reduced specific staining of 0.5+, whereas specific staining to 3+ was observed for the D13150-D13155 pair. The replacement in D13150 of 2 A's with 2 D's and of 2 T's with 2 U_{S}'s (D13171) resulted in improved specific binding compared to D13106. This modified PNA1 was now able to bind specifically to D13148 with a score of 2.5+, and also bound to D13155.

This experiment clearly demonstrates the use of PNA pairs in the present invention. Furthermore, it shows the ability of fine tuning the specific binding by introducing base substitutions using either natural as well as non-natural bases.

### Example 26

### Test of Cross Reactivity

The two PNA-pairs, D13150-D13155 and D13161-D13159, were tested for cross-reactivity. Tonsil tissues were taken through the steps 1-4, 6-8, 10, and 13-17. The concentration of conjugates used was 0.16 μM for PNA1 and 0.1 μM for PNA2.

As apparent from Table 11, PNA1 D13150 did not cross react with PNA2 D13159, but PNA1 D13161 cross reacted with PNA2 D13155. We therefore excluded the PNA pair D13161-D13159 due to the cross-reaction between D13161 and D13155. No non-specific staining was observed.

TABLE 11
Test of specific binding and cross reactivity

PNA2:

| PNA1: | D13155 | D13159 |
|---|---|---|
| D13150 | 2.0 | 0 |
| D13161 | 1 | 1.5 |

### Example 27

### Test of Cross Reactivity

Three PNA-pairs, D14083-D14079, D14102-D14089 and D14120-D14114 were tested for cross-reactivity. Tonsil tissues were taken through the steps 1-4, 6-8, 10, and 13-17. The concentration of the conjugates used was 0.08 μM for PNA1 and 0.05 μM for PNA2.

The PNA conjugates listed in Table 12 only bound to their complementary partner and did not cross react to any of the other PNA conjugates tested. No non-specific staining was observed. See Table 12 below.

TABLE 12
Test of specific binding and cross reactivity

| | PNA2: | | |
|---|---|---|---|
| PNA1: | D14079 | D14089 | D14114 |
| D14083 | 1.5 | 0 | 0 |
| D14102 | 0 | 1 | 0 |
| D14120 | 0 | 0 | 1.5 |

### Example 28

Two PNA pairs, D14083-D14079 and D14096-D14089, were tested at different PNA2 concentrations for the purpose of determining the optimal concentration of PNA2 conjugates. The concentrations used were 0.08 μM for PNA1 conjugates and 0.025; 0.05; 0.1 and 0.2 μM for PNA2 conjugates.

Tonsil tissues were taken through the steps 1-4, 6-8, 10, and 13-17. The optimal concentration of the PNA2 conjugate was 100 nM. See Table 13 below.

TABLE 13
Determination of PNA2 conjugate concentration.

| | 1% GA | | | | | |
|---|---|---|---|---|---|---|
| | fixation of | | Specific staining | | | |
| PNA1 | PNA1 | PNA2 | 0.025 μM | 0.05 μM | 0.1 μM | 0.2 μM |
| D14083 | − | D14079 | 1.5 | 2 | 3 | 2.5 |
| D14096 | − | D14089 | 0.5 | 1 | 2 | 1.5 |

Tonsil tissues were taken through the steps 1-4, 6-8, 10, 11, and 13-17. Step 11 was omitted for tissues not fixed with 1% GA.

Fixation of PNA1 conjugates with 1% GA resulted in a stronger specific staining than without fixation and the optimal concentration of the PNA2 conjugate was now determined to be 50 nM. See Table 14 below.

TABLE 14
Effect of 1% GA fixation on the determination of PNA2 conjugate
concentration.

| | 1% GA | | | | | |
|---|---|---|---|---|---|---|
| | fixation of | | Specific staining | | | |
| PNA1 | PNA1 | PNA2 | 0.025 μM | 0.05 μM | 0.1 μM | 0.2 μM |
| D14083 | − | D14079 | 2 | 2 | 2.5 | 2.5 |
| D14083 | + | D14079 | 2.5 | 3 | 2.5 | 3 |

### Example 29

### 2-Layer Versus 3-Layer PNA Systems

This example shows the results of using a 3-layer PNA system employing a PNA¹-PNA²/Dextran conjugate adaptor unit to link the PNA1 and PNA2 conjugates together. Tonsil tissues were taken through the steps 1-4, 6-8, and 10-17. Step 11 was omitted for tissues not to be fixed with 1% GA. The concentration of the conjugates used was 0.08 μM for PNA1, 0.1 μM (calculated based on PNA¹) for PNA¹-PNA² and 0.05 μM for PNA2.

Table 15 shows that a 3-layer system resulted in a stronger specific staining intensity in comparison with a 2-layer system. No non-specific staining was observed.

TABLE 15
Improvement of staining intensity by using 3 layers

| | 1% GA | | | Specific |
|---|---|---|---|---|
| | fixation of | | | staining |
| PNA1 | PNA1 | PNA-PNA | PNA2 | intensity |
| D14096 | + | D14104 | D14079 | 2 |
| D14096 | − | D14104 | D14079 | 2 |
| D14096 | − | − | D14089 | 1 |

The introduction of a fixation step after the application of PNA1 resulted in an increase in specific staining. The specific staining was increased with 1+ score in the 3-layer system as shown in Table 16. Surprisingly no non-specific staining was observed despite the use of a multi-layer PNA-system.

TABLE 16
Improvement of specific staining in a 3-layer system by fixation

| | 1% GA | | | Specific |
|---|---|---|---|---|
| | fixation of | | | staining |
| PNA1 | PNA1 | PNA-PNA | PNA2 | intensity |
| D14102 | + | D14119 | D14079 | 3 |
| D14102 | + | D14106 | D14079 | 3 |
| D14102 | + | D14104 | D14079 | 3 |
| D14102 | − | D14119 | D14079 | 2 |
| D14102 | − | D14106 | D14079 | 2 |
| D14102 | − | D14104 | D14079 | 2 |

### Example 30

### Test of 3-Layer PNA Systems using Different PNA¹-PNA² Concentrations in the Presence or Absence of Fixation

Tonsil tissues were taken through the steps 1-4, 6-8, and 10-17 above. Step 11 was left out for the tonsil tissues, which were not going to be fixed with 1% GA. The concentration of the conjugates in the table was 0.08 μM for PNA1, 0.025, 0.05, 0.1 and 0.2 μM for PNA¹-PNA² (based on [PNA¹]), and 0.05 μM for PNA2.

Fixation of PNA1 increased the specific staining intensity with 0.5+ to 1+ score.

TABLE 17
The effect of using different PNA¹-PNA² concentrations.

| | | Specific staining intensity | | | |
|---|---|---|---|---|---|
| | | at various PNA¹-PNA² concentration | | | |
| PNAs | 1% GA | 0.025 μM | 0.05 μM | 0.1 μM | 0.2 μM |
| D14102 | + | 2.5 | 2.5 | 3 | 3 |
| D14119 | − | 2 | 2 | 2 | 2 |
| D14079 | | | | | |
| D14096 | + | 1.5 | 2 | 2 | 3 |
| D14104 | − | 1 | 1.5 | 2 | 2.5 |
| D14079 | | | | | |

### Example 31

### Effect of using different Concentrations of Glutardialdehyde (GA) for Fixation of PNA1

A 2-layer PNA test system was employed to study the effect of using different concentrations of GA. Tonsil tissues were taken through the steps 1-4, 6-8, 10, 11 and 13-17. In step 11, the concentration of GA used was 0.1%, 0.3% and 1.0% respectively. The PNA pair, D14083-D14079, was used at concentrations of 0.08 μM for PNA1 and 0.05 μM for PNA2. After fixation of PNA1 conjugates, the tissues were processed with one of three treatments listed in Table 18.

The specific staining in the 2-layer PNA system was improved when the PNA1 conjugate was fixed with at least 0.3% GA, even when the tissues were boiled in target retrieval buffer in microwave oven for 10 min. This shows the possibility of including a strong cross linking step to the procedure. The cross linking allows a harsh treatment with no sacrifice to the staining result.

TABLE 18
Staining intensity at different Glutardialdehyde (GA) concentration.

| | Specific staining intensity | | |
|---|---|---|---|
| Treatment after GA-fixation of PNA1 | 0.1% GA | 0.3% GA | 1.0% GA |
| Wash in TBST at RT for 20 min. | 1.5 | 1.5 | 1.5 |
| Wash in TBST at 65° C. for 10 min. | 1.0 | 1.5 | 1.5 |
| Target retrieval (K5204) in MW | 1.0 | 1.5 | 1.5 |
| oven for 10 min. | | | |

### Example 32

### Comparison of a PNA-Based Detection System with an EnVision™ Based Detection System

Tonsil tissues were taken through the steps 1-4, 6, 7, 9-10, and 13-17. The concentration of the conjugates was 0.08 μM for PNA1, D12102 and 0.05 μM for PNA2, D12094. A negative Ig control, mouse IgG1 (DakoCytomation code No. X0931) diluted 1:300 in S2022 was included in the protocol for PNA conjugates. The EnVision™-based detection system, K5007, was used in parallel with the PNA based detection system.

The specific staining intensities obtained for the three antibodies tested and visualized with either the D12102-D12094 PNA-pair or K5007 are shown in Table 19. The PNA based system showed in general a stronger specific staining in comparison with the reference K5007.

TABLE 19
Comparison between two indirect detection systems.

| | | Specific staining intensity | |
|---|---|---|---|
| Primary | Dilution of | PNA based | EnVision ™-based |
| antibody | primary antibody | detection system | detection system |
| M3515 | 1:300 | 3 | — |
| | 1:900 | 2.5 | 2 |
| | 1:1600 | 1.5 | — |
| M7240 | 1:400 | 3 | — |
| | 1:1200 | 2.5 | 2 |
| | 1:2400 | 1.5 | — |
| M0755 | 1:2000 | 3 | — |
| | 1:8000 | 2.5 | 2 |
| | 1:14000 | 1.5 | — |
| X0931 | 1:300 | 0 | 0 |

### Example 33

### Recognition of a Conjugated Primary Antibody by another Detection System

Tonsil tissues were taken through the steps 1-4, 6, 7, 10, 11, 1S and 14-17 above. 20 μL of PNA1, respectively D14122 and D14128 (0.08 and 0.3 μM) were applied. Slides were cover slipped during incubation with PNA1. Then 200 μL PNA2, D14079 (0.1 μM) was applied. Samples were incubated with K5007 GaM:HRP complex for 30 min in parallel with PNA2 conjugates in step 13. As a further control and for comparison, tonsil tissues were taken through steps 1-4 and 6-8 using uncomplexed anti-BCL2, M0887, diluted 1:100 to a concentration of 0.015 μM in S2022 as primary antibody, and visualized by incubation with K5007 GaM:HRP complex for 30 min. as an alternative to the PNA2 conjugates in step 13. These slides were then taken through steps 14-17.

Table 20 summarizes the staining results. When preparing PNA conjugates with multiple PNAs, here illustrated by PNA1, the PNAs remained accessible for hybridization to complementary PNAs comprised in components further comprising dextran and enzymes. Conjugates comprising more PNA did not necessarily show improved specific staining. Instead, the amount of staining peaked and then fell as the PNA to dextran ratio increased. For example, samples incubated with D14126 scored 1.0+, those with D14128 scored 2.5+, and those with D14122 scored 2.0+. Thus, D14128, with a PNA:Dextran ratio of about six, gave a stronger signal than both D14122 with a PNA:Dex ratio of about nine as well as D14126 with a PNA:Dex ratio of about three. When preparing PNA conjugates with multiple PNAs, here illustrated by PNA1, the PNAs remained accessible for hybridization to complementary PNAs comprised in components further comprising dextran and enzymes.

This experiment also illustrates that the conjugation of multiple PNAs to an antibody may reduce the recognition of the antibody by a secondary antibody:enzyme complex. Samples treated with anti-BCL2 antibody conjugated with PNA1 resulted in specific staining intensities of 2.5+ with PNA2 and 1.5+ with K5007 respectively. Samples treated with free anti-BCL2 and K5007 showed a 3 + score. The signal obtained with K5007 decreased with the number of PNA in the PNA1 conjugate.

TABLE 20
Significance of the amount of PNA in PNA1 conjugates on
specific staining intensity.

| | | Specific | |
|---|---|---|---|
| | | staining | |
| | | intensity with | Specific staining |
| | PNA/ | PNA2, | intensity with |
| | dextran | D14079 | K5007 |
| PNA1, D14126 | 2.9 | 1.0 | — |
| 0.3 μM | | | |
| PNA1, D14128 | 5.6 | 2.5 | 1.5 |
| 0.3 μM | | | |
| PNA1, D14128 | 5.6 | 0.5 | 1.0 |
| 0.08 μM | | | |
| PNA1, D14122 | 9.5 | 2 | 1.5 |
| 0.3 μM | | | |
| PNA1, D14122 | 9.5 | 0.5 | 0 |
| 0.08 μM | | | |
| Unconjugated, | — | — | 3.0 |
| M0887 | | | |

### Example 34

### Test of Non-Specific Binding due to PNA2 Conjugates

Tonsil tissues were taken through the steps 1-5, 7, 13 and 14-17 above. The PNA2 conjugates tested are listed in Table 21. Replacing 2 D's in 12120 with 2 A's (D13106) reduced the non-specific staining from 3+ to 1+ (at 0.05 μM PNA2). When 2 G's in D13106 were replaced with 2 G_{S}'s (D13148), the non-specific staining was reduced from 1+ to 0. Reintroducing 2 D's in D13155 instead of 2 A's (D13148) increased the non-specific staining from 1.5+ to 2.0+ (at 0.2 μM PNA). Both D12120 and D13155 had 2 D's in the sequence, but the non-specific staining in D13155 did not reach the same level as in D12120, probably due to the 2 G_{S}'s in D13155. The same effect of non-natural bases was seen when comparing D12120 with D14133: the non-specific staining in D14133 did not reach the same level as in D12120, this time probably due to 2 inosines (I)'s in D14133. The replacement of 2 G's in D13122 with 2 G_{S}'s (D13159) reduced the non-specific staining from 3+ to 0 (at a PNA concentration of 0.2 μM). Substitution of 1 G in D14110 with 1 Gs (D14089), reduced the non-specific staining from 1+ to 0. Equally, 1 G in D14086 was replaced with 1 G_{S} (D14089), resulting in a reduction of the non-specific staining from 3+ to 0 (at a PNA concentration of 0.2 μM).

The non-specific background staining generated by the PNA2 conjugates could be subtly fine tuned, and ultimately completely eliminated, by base substitution using both natural as well as non-natural bases. The level of background correlated directly to the DNA/RNA affinity of the PNAs of the conjugates. This was surprising, as the conjugates have a molecular weight around 500 kDa, and the changes in the PNAs necessary to bring about strongly reduced background in some cases were as little as one D to A substitution (thus removing one potentially hydrogen bonding amino group) or one G to G_{S} substitution (introducing a single carbonyl to thiocarbonyl change).

TABLE 21
Non-specific staining intensities of due to
PNA2 conjugates.

| | Non-specific staining | | |
|---|---|---|---|
| | intensity | | |
| | 0.2 μM | 0.05 μM | |
| PNA2 | PNA2 | PNA2 | Sequence |
| D12120 | 3 | 3 | TCD DGG TAC A |
| D13106 | 2.5 | 1 | TCA AGG TAC A |
| D13148 | 1.5 | 0 | TCA AG_{S}G_{S} TAC A |
| D13155 | 2 | 0 | TCD DG_{S}G_{S} TAC A |
| D14133 | 0.5 | 0 | TCD DII TAC A |
| D13122 | 3 | 2 | CTA AGG TCA A |
| D13159 | 0 | 0 | CTA AG_{S}G_{S} TCA A |
| D14086 | 3 | 2.5 | U_{S}CG_{S} G_{S}DD TU_{S}D GDC |
| D14089 | 0 | 0 | CU_{S}G_{S} G_{S}DD TU_{S}D G_{S}DC |
| D14110 | 1 | 0 | DGT CG_{S}D DG_{S}G U_{S}CU_{S} |
| D14114 | 0 | 0 | DG_{S}T CG_{S}D DG_{S}G U_{S}CU_{S} |

### Example 35

### Use of Fluorescein as a Molecular Label

Tonsil tissues were taken through the steps 1-4, 6-8, 10, and 13 (PNA2 was conjugated with fluorescein) and 16 (the slides were mounted with Vectashield containing DAPI). The concentration used for the conjugates was 0.08μM for PNA1, D13171 and 0.02, 0.05, 0.1, and 0.2 μM for PNA2, D14008 (TCD DG_{S}G_{S}TAC A). The slides were evaluated using a fluorescence microscope at 40× and 100× magnifications.

The specific staining was satisfactory. This experiment demonstrated the possibility of making PNA-fluorescein conjugates and illustrated the application of the present invention with a fluorescein as detectable label.

### Example 36

### Test of a PNA Pair with 10 Antibodies

A multi-block containing tissue from mammalian carcinoma, kidney, colon and two tonsils was taken through the steps 3-4, 6-8, 10, 13-17. In step 8, ten different primary antibodies and a negative control mouse IgG1 were used. (See table 22.) The concentration of the conjugates used was 0.08 μM for PNA1, D12102 and 0.05 μM for PNA2, D12094. Visualization of primary reagents by the K5007 detection kit was performed in parallel according to the manufacturer's instructions. Two primary antibodies targeting membranous targets were used: Monoclonal Mouse Anti-Human CD20cy (DakoCytomation code No. M0755) and Monoclonal Mouse Anti-Human Epithelial Membrane Antigen (DakoCytomation code No. M0613). Five primary antibodies targeting cytoplasmic targets were used: Monoclonal Mouse Anti-Human Cytokeratin (DakoCytomation code No. M3515), Monoclonal Mouse Anti-Human Desmin (DakoCytomation code No. M0760), Monoclonal Mouse Anti-Human CD68 (DakoCytomation code No. M0876), Monoclonal Mouse Anti-Human BCL2 (DakoCytomation code No. 0887) and Monoclonal Mouse Anti-Human CD45 LCA (DakoCytomation code No. M0701). Three primary antibodies targeting nuclear targets were used: Monoclonal Mouse Anti-Human Estrogen Receptor (DakoCytomation code No. M7047), Monoclonal Mouse Anti-Human Ki-67 Antigen (DakoCytomation code No. M7240) and Monoclonal Mouse Anti-Human p27 (DakoCytomation code No. M7203). The primary antibodies, all products from DakoCytomation, were diluted in S2022 as indicated in Table 22.

This example shows the extended use of the PNA system employing several primary antibodies. Furthermore, in the majority of cases using the 10 primary antibodies, visualization by the PNA based detection system demonstrated an improved staining as compared to the reference K5007.

TABLE 22
Comparison of the PNA based detection system with EnVision ™

| Primary | Antibody | Specific staining intensity | |
|---|---|---|---|
| Antibody | Dilution | The PNA system | The K5007 reference |
| M3515 | 1:900 | 2 | 1.5 |
| M0876 | 1:800 | 2 | 2 |
| M0887 | 1:500 | 2 | 2 |
| M0760 | 1:1200 | 1.5 | 1.5 |
| M0701 | 1:1600 | 3 | 2 |
| M0755 | 1:8000 | 1.5 | 1.5 |
| M0613 | 1:6400 | 2.5 | 1.5 |
| M7047 | 1:200 | 3 | 2.5 |
| M7240 | 1:1200 | 2 | 1.5 |
| M7203 | 1:400 | 2.5 | 1.5 |

### Example 37

### Standard Synthesis of AP-DexVS70-PNA Conjugate

Alkaline Phosphatase (“AP”) (from Calf Intestine, EIA grade) was dialyzed overnight against 2 mM HEPES, pH 7.2; 0.1M NaCl; 0.02 mM ZnCl₂. Dextran (molecular weight 70 kDa) was activated with divinylsulfone to a degree of 92 reactive groups per dextran polymer (DexVS70).

The three components below were mixed together and placed in a water bath at 40° C. for 30 minutes.

| | |
|---|---|
| 192.0 μL DexVS70 | 13.7 nmol |
| 41.0 μL PNA | 41 nmol PNA dissolved in H₂O |
| 6.0 μL 1M NaHCO₃ | |

108.0 μL of the DexVS70-PNA conjugate was taken out and added to a mixture of:

| | |
|---|---|
| 160.0 μL AP | 43.4 nmol |
| 7.7 μL 1M NaHCO₃ | |
| 30.6 μL 20 mM Hepes, pH 7.2; | |
| 1M NaCl; 50 mM MgCl₂; 1 mM ZnCl₂ | |

The mixture was placed in a water bath at 40° C. for 3 hours. Quenching was performed by adding 30.6 μL of 0.1 M ethanolamine and letting the mixture stand for 30 minutes in water bath at 40° C. The product was purified on FPLC with: Column Superdex-200, buffer: 2 mM HEPES, pH 7.2; 0.1M NaCl; 5 mM MgCl₂; 0.1 mM ZnCl₂. Two fractions were collected, one with the product and one with the residue.

In comparison to the experiment described above, another conjugate was made with extended conjugation time. The three components below were mixed together and placed in a water bath at 40° C. for 30 minutes.

| | |
|---|---|
| 192.0 μL DexVS70 | 13.7 nmol |
| 41.0 μL PNA | 41 nmol PNA dissolved in H₂O |
| 6.0 μL 1M NaHCO₃ | |

108.0 μL of the DexVS70-PNA conjugate was taken out and added to a mixture of:

| | |
|---|---|
| 160.0 μL AP | 43.4 nmol |
| 7.7 μL 1M NaHCO₃ | |
| 30.6 μL 20 mM Hepes, pH 7.2; | |
| 1M NaCl; 50 mM MgCl₂; 1 mM ZnCl₂ | |

The mixture was placed in a water bath at 40° C. for 5 hours. Quenching was performed by adding 30.6 μL 0.1M Ethanolamine and letting the mixture stand for 30 minutes in water bath at 40° C. Purification of the product on FPLC: Column Superdex-200, buffer: 2 mM Hepes, pH 7.2; 0.1M NaCl; 5 mM MgCl₂; 0.1 mM ZnCl₂. Two fractions were collected: One with the product and one with the residue.

Relative absorbance PNA(Flu) (ε_{500 nm}=73000M⁻¹) and AP(ε_{278 nm}=140000M⁻¹. Corrected for absorbance from PNA at 278 nm, this correction factor is due to the specific PNA and it is calculated: 278/500nm) was used to calculate the average conjugation ratio of PNA, AP and DexVS70.

AP-DexVS70-PNA, 3 hrs:

PNA/DexVS70: 1.8

AP/DexVS70: 1.8

AP-DexVS70-PNA, 5 hrs:

PNA/DexVS70: 2.0

AP/DexVS70: 2.4

Due to these results, it is recommended to follow a procedure in which the conjugation time (AP+DexVS70-PNA) is 5 hours.

### Example 38

### Synthesis and IHC Testing of an Antibody-PNA Conjugate

Part A. Testing Different Ratios of Linker to Antibody

Materials: Antibody: CD 45 dialized overnight against 0.01 M Hepes 0.1 M NaCl pH=7.2. SMCC: Succinimidyl-4(N-maleimidomethyl)cyclohexan-1-carboxylate molw. 334.33. PNA: Acetyl- L₃₀-GTP-TAA-TTP-PAG-L₁₅₀-Lys(Cys)

Test 1

10 nmol CD45 was dissolved in 161 μL 0.01 M Hepes 0.1 M NaCl pH=7.2. 250 nmol SMCC was dissolved in 8 μL NMP. The above components were mixed and placed in a water bath at 30° C. for 60 minutes. The mixture was purified on a mini-prep column (Sephadex G-25) with 0.01 M Hepes 0.1 M NaCl pH=7.2 as eluent. Fractions of 0.3 mL. By measuring absorbance at 278 nm three fractions containing the product (58%) were identified. These three fractions were added to 100 nmol of a lyophilised PNA. Then 1 μL 5% Di-Sodium-EDTA/water was added and the solution was mixed until dissolved and placed in a water bath at 30° C. for 30 minutes. Quenching was performed by adding 2 mg of Cysteine. Water bath 30° C. for 30 minutes.

The product was purified on FPLC: Column SUPERDEX®-75, Buffer 0.01 M Hepes 0.1 M NaCl pH 7.2. The fraction with the product was collected. Relative absorbance between PNA (ε_{260 nm}) and antibody (ε_{278 nm}) was used to calculate the average conjugation ratio of PNA and antibody. PNA/CD45: 5.2. Yield 39% based on antibody.

### Test 2

10 nmol CD45 was dissolved in 161 μL 0.01 M Hepes 0.1 M NaCl pH=7.2. 150 nmol SMCC was dissolved in 5 μL NMP. The above components were mixed and placed in a water bath at 30° C. for 60 minutes. The mixture was purified on a mini-prep column (SEPHADEX® G-25) with 0.01 M Hepes 0.1 M NaCl pH=7.2 as eluent. Fractions of 0.3 mL. By measuring absorbance at 278 nm three fractions containing the product (74%) were identified.

These three fractions were added to 100 nmol of a lyophilized PNA. Then 1 μL 5% Di-Sodium-EDTA/water was added and the solution was mixed until dissolved and placed in a water bath at 30° C. for 30 minutes. Quenching was performed by adding 2 mg of cysteine. Water bath 30° C. for 30 minutes.

The product was purified on FPLC: Column Superdex-75, Buffer 0.01 M Hepes 0.1 M NaCl pH 7.2. The fraction with the product was collected. Relative absorbance between PNA (ε_{260 nm}) and antibody (ε_{278 nm}) was used to calculate the average conjugation ratio of PNA and antibody. PNA/CD45: 3.4. Yield 55% based on antibody.

### IHC Test of Conjugates

A later IHC test showed that PNA/CD45 in Test 2 gave a higher score than the one in Test 1. This brought us to the conclusion that the ratio between CD45/SMCC/PNA should be 10/150/100.

Part B. Test of Different Conjugation Times—Antibody and Linker

Materials: Antibody: GAM (goat-anti-mouse) dialysed overnight against 0.1 M NaCl. SMCC: Succinimidyl-4(N-maleimidomethyl)cyclohexan-1-carboxylate molw. 334.33. Flu-Link: Flu-L₉₀-Lys(Flu)-L₃₀-Lys(Cys)

### Test 1

20 nmol GAM was dissolved in 402 μL 0.01 M Hepes 0.1 M NaCl pH=7.2. 400 nmol SMCC dissolved in 13 μL NMP. The above components were mixed and placed in a water bath at 30° C. for 1 hour. 207 μL of the mixture was purified on a mini-prep column (SEPHADEX® G-25) with 0.01 M Hepes 0.1 M NaCl pH=7.2 as eluent. Fractions of 0.3 mL were taken. By measuring absorbance at 278 nm, three fractions containing the product (79%) were identified. These three fractions were added to 200 nmol of a lyophilized Flu-Link. Then 1 μL 5% di-sodium-EDTA/water was added and the solution was mixed until dissolved and placed in a water bath at 30° C. overnight. Quenching was performed by adding 2 mg of Cysteine. Water bath 30° C. for 30 minutes.

The product was purified on FPLC: Column SUPERDEX®-75, Buffer 0.01 M Hepes 0.1 M NaCl pH 7.2. The fraction with the product was collected. Relative absorbance between Flu-Link (ε_{498 nm}) and antibody (ε_{278 nm}) was used to calculate the average conjugation ratio of PNA and antibody. Flu-Link/GAM: 7.1. Yield 55% based on antibody.

### Test 2

20 nmol GAM was diluted with 402 μL 0.01 M Hepes 0.1 M NaCl pH=7.2. 400 nmol SMCC was dissolved in 13 μL NMP. The above components were mixed and placed in a water bath at 30° C. for 2 hours. The rest of the synthesis and purification was done in exactly the same procedure as for 1 hour. There was a 64% yield of GAM/SMCC before adding the Flu-Link. Flu-Link/GAM: 8.7. Yield 33% based on antibody.

Part C. Test of Different Conjugation Times—Fluorophore and Linker

Materials: Antibody: GAM dialysed overnight against 0.1 M NaCl. SMCC: Succinimidyl-4(N-maleimidomethyl)cyclohexan-1-carboxylate molw. 334.33. Flu-Link: Flu-L₉₀-Lys(Flu)-L₃₀-Lys(Cys)

### Test 1

20 nmol GAM was dissolved 378 μL 0.01 M Hepes 0.1 M NaCl pH=7.2. 400 nmol SMCC was dissolved in 13 μL NMP. The above components were mixed and placed in a water bath at 30° C. for 1 hour. The mixture was divided in two and purified on two mini-prep columns (SEPHADEX® G-25) with 0.01 M Hepes 0.1 M NaCl pH=7.2 as eluent. Fractions of 0.3 mL were taken. By measuring absorbance at 278 nm, three fractions from each column containing the product (76% in all) were identified. These six fractions were pooled, divided in two, and each added to 200 nmol of a lyophilized Flu-Link. Then 1 μL 5% di-sodium-EDTA/water was added and the solution was mixed until dissolved and placed in a water bath at 30° C., one for 30 minutes, the other for 60 minutes. Quenching was performed by adding 2 mg of cysteine. Water bath 30° C. for 30 minutes.

The product was purified on FPLC: Column SUPERDEX®-75, Buffer 0.01 M Hepes 0.1 M NaCl pH 7.2. The fractions with the product from each purification were collected. Relative absorbance between Flu-Link (ε_{498 nm}) and antibody (ε_{278 nm}) was used to calculate the average conjugation ratio of PNA and antibody. 30 minutes conjugation Flu-Link/GAM: 7.0 Yield 55% based on antibody. 60 minutes conjugation Flu-Link/GAM: 6.9 Yield 52% based on antibody. The above results show that 30 minutes conjugation between GAM/SMCC and Flu-Link is sufficient.

### Example 39

### Additional Tests of 2 and 3 Layer Visualization Systems

Primary mouse antibody M7240 (Dako) targeting MIB-1 was diluted to final 1:150 in S2022 buffer (Dako) and applied on a multi tissue section. Following 10 minutes incubation at RT the section was washed 5 minutes using 10× diluted S3006 buffer (Dako).

Goat-anti-mouse secondary antibody conjugated with dextran and a first PNA sequence (GaM-dex-PNA1 (218-117)) was diluted to final concentration of 0.08 μM (based on dextran) in BP-HEPES-buffer (1.5% BSA, 3% PEG, 0.15M NaCl, 10 mM HEPES, pH 7.2) and was applied to the section. Following 10 minutes incubation at room temperature (RT), the section was washed 5 minutes using 10× diluted S3006 (Dako). The sections were rinsed in deionized water. Following 10 minutes incubation in 0.5% glutaraldehyde at RT, the sections were rinsed in deionized water and washed 5 minutes using 10× diluted S3006 (Dako).

An adaptor unit comprising dextran coupled to two different PNA sequences, one complementary to PNA1 above (PNA2) and another not complementary to PNA1 (PNA3), called PNA2-dex-PNA3 (218-057) was diluted to a final concentration of 0.05 μM (dextran) in BP-HEPES-buffer (1.5% BSA, 3% PEG, 0.15M NaCl, 10 mM HEPES, pH 7.2) and was applied to the section. Following 10 minutes incubation at RT, the section was washed 5 minutes using 10× diluted S3006 (Dako). Next, a conjugate of a PNA4, complementary to PNA3 above, dextran, and the detectable label alkaline phosphatase (PNA4-dex-AP (209-177)) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2), and was applied. Following 10 minutes incubation at RT, the sections were washed 5 minutes using 10× diluted S3006 (Dako).

“Permanent Red working solution” (an aqueous Tris buffer with naphthol-phosphate and a diazonium dye; K0640 Dako) was prepared and then applied. Following 10 minutes incubation, the section was washed 5 minutes using 10× diluted S3006 (Dako). Finally the sections were counter stained 5 minutes using haematoxylin S3301 (Dako), rinsed in deionzed water, washed 3 minutes in wash buffer, and mounted in Faramount S3025 (Dako). Result: MIB-1=1+/0.5+.

### Example 40

Primary rabbit antibody A0452 (Dako) targeting CD3 was diluted to final 1:100 in S2022 (Dako) and applied on a multi tissue section. Following 10 minutes incubation at RT the sections were washed 5 minutes using 10× diluted S3006 (Dako).

Then, a goat-anti-rabbit secondary antibody coupled to dextran and a first PNA sequence, PNA2a (GaR-dex-Alexander (209-127)) was diluted to final concentration of 0.08 μM (dextran) in BP-HEPES-buffer (1.5% BSA, 3% PEG, 0.15M NaCl, 10 mM HEPES, pH 7.2) and was applied. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

Next, a complementary PNA coupled to dextran and detectable label alkaline phosphatase (AP) (PNA2b-dex-AP (209-177) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the section was washed 5 minutes using 10× diluted S3006 (Dako).

Permanent Red working solution (K0640 Dako) was prepared and was applied. Following 10 minutes incubation the sections were washed 5 minutes using 10× diluted S3006 (Dako). Finally the sections were counter stained 5 minutes using haematoxylin S3301 (Dako), rinsed in deionzed water, washed 3 minutes in wash buffer, and mounted in Faramount S3025 (Dako). Result: CD3 specific staining=1.5+ compared to non-specific background staining of 1+. The order of detection affects the staining result.

### Example 41

### Detecting Two Targets in a Sample

General Procedural Note: Before conducting the detection experiment on formalin-fixed, paraffin-embedded (FPPE) tissue sections, the specimen should be deparaffinized (dewaxed), rehydrated, and blocked for endogenous peroxidase activity. Some specimens should be subjected to target retrieval using heat or enzyme digestion. Following target retrieval, the specimens should be rinsed gently with wash buffer.

Part A. Two-Layer Detection Experiment using Secondary Antibody Probes

In this experiment, a mouse primary antibody was used as a primary binding agent for a specific target in a tissue sample. That antibody was then recognized by a goat-anti-mouse-dextran-PNA conjugate recognition unit. A different primary antibody, a rabbit antibody, was used as a primary binding agent for a different target in the sample. That antibody was recognized by a goat-anti-rabbit-dextran-PNA recognition unit. One reaction was visualized by a PNA-dextran-HRP (horse-radish peroxidase) detection unit and the other reaction was visualized by a PNA-dextran-AP (alkaline phosphatase) detection unit. PNA sequences 1 and 2 and sequences 3 and 4, respectively, specifically hybridize to each other.

Primary mouse antibody M3515 (Dako) targeting Cytokeratin and primary rabbit antibody 20311 (Dako) targeting S100 were diluted 1:50 and 1:100 in S2022 buffer (Dako), respectively. The antibodies were applied simultaneously on multi tissue sections. Following 10 minutes incubation at RT the sections were washed 5 minutes using 10× diluted S3006 buffer (Dako). Goat-anti-mouse-dextran-PNA1 (GaM-dex-PNA1) and goat-anti-rabbit-dextran-PNA3 (GaR-dex-PNA3) were both diluted to a final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at room temperature (RT), the sections were washed 5 minutes using 10× diluted S3006 (Dako). The sections were rinsed in deionized water.

The samples were then incubated for 10 minutes in 0.5% glutaraldehyde at RT and then rinsed in deionized water and washed 5 minutes using 10× diluted S3006 (Dako). PNA2-dex-HRP and PNA4-dex-AP were both diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2). The two conjugates were applied simultaneously on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution (an aqueous imidazole buffer with hydrogen peroxide and DAB) was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: Cytokeratin=HRP=3+ specific staining and 0 background staining, S100=AP=3+ specific staining and 0 background staining. The order of detection affects the staining result. If detection method 1 is used then Permanent Red dominates. If detection method 2 is used then DAB+ dominates.

Part B. Two-Layer Detection Experiment using Antibodies as Probes

Primary mouse antibody M3515 (Dako) targeting Cytokeratin and primary rabbit antibody Z0311 (Dako) targeting S100 were diluted to final 1:50 and 1:400 in S2022 (Dako), respectively. The antibody mixture was applied on multi-tissue sections. Following 10 minutes incubation at RT the sections were washed 5 minutes using 10× diluted S3006 (Dako). GaM-dex-PNA1 (209-149) and GaR-dex-PNA2 (209-127) were both diluted to final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections.

Following 10 minutes incubation at RT the sections were washed 5 minutes using 10× diluted S3006 (Dako). The sections were rinsed in deionized water. Following 10 minutes incubation in 1% glutaraldehyde at RT the sections were rinsed in deionized water and washed 5 minutes using 10× diluted S3006 (Dako). PNA2-dex-HRP (209-157) and PNA4-dex-AP (209-177) were both diluted to final concentration of 0.05 μM/dex in BAP-HEPES-buffer (1.5% BSA, 3% PEG, 0.15M NaCl, 0.05% 4-aminoantipyrin, 10 mM HEPES, pH 7.2). The two conjugates were applied simultaneously on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted 53006 (Dako). Then DAB+ working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: Using detection method 1: Cytokeratin=HRP=2.5+ specific staining and 0 background staining, S100=AP=2.5+ specific staining and 0 background staining. Using detection method 2: Cytokeratin=HRP=3+ specific staining and 0.5+ background staining, S100=AP=3+ specific staining and 0 background staining. The order of detection affects the staining result.

### Example 42

### Further 2 and 3 Layer Systems for Detection of Multiple Targets

Part A. Combined Two and Three-Layer System

In this example, a mouse antibody primary binding agent was recognized by a GaM-dex-PNA1 and a rabbit antibody primary binding agent was recognized by GaR-dex-PNA2. One reaction was detected by a PNA-dex-Enzyme1 conjugate and the other by a PNA-dex-PNA adaptor unit and then a PNA-dex-Enzyme2 conjugate. PNA1 recognizes PNA2 while PNA3 recognizes PNA4. The enzymes used were HRP and AP, bringing along respectively a brown and red end-product within the same tissue section. The PNA-dex-PNA adaptor unit adds a third layer to the detection system.

Primary mouse antibody M7240 (Dako) targeting MIB-1 and primary rabbit antibody A0452 (Dako) targeting CD3 were diluted to final 1:150 and 1:100 in S2022 (Dako), respectively. The antibody mixture was applied on multi tissue sections. Following 10 minutes incubation at RT the sections were washed 5 minutes using 10× diluted S3006 (Dako).

GaM-dex-PNA1 (218-117) and GaR-dex-PNA3 (209-127) were both diluted to final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA4-dex-HRP (218-021) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). PNA2-dex-PNA3 (218-057) amplification unit was diluted to final concentration of 0.05 μM (dextran) in BP-HEPES-buffer (1.5% BSA, 3% PEG, 0.15M NaCl, 10 mM HEPES, pH 7.2) and applied on the sections. Following 10 minutes incubation at RT the sections were washed 5 minutes using 10× diluted S3006 (Dako). PNA4-dex-AP (209-177) was diluted to final concentration of 0.05 μM/dex in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: CD3=2-layer experiment=HRP=3+ specific and 0 background staining, MIB-1=3-layer experiment=AP=2+ specific and 1.5+ background staining. The order of detection affects the staining result.

Part B.

Primary mouse antibody M7240 (Dako) targeting MIB-1 and primary rabbit antibody A0452 (Dako) targeting CD3 were diluted to final 1:150 and 1:100 in S2022 (Dako), respectively. The antibody mixture was applied on multi tissue sections. Following 10 minutes incubation at RT the sections were washed 5 minutes using 10× diluted S3006 (Dako).

GaM-dex-PNA1 (218-117) and GaR-dex-PNA3 (209-127) were both diluted to final concentration of 0.08 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA4-dex-AP (209-177) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako). PNA2-dex-PNA3 (218-057) was diluted to final concentration of 0.05 μM (dextran) in BP-HEPES-buffer (1.5% BSA, 3% PEG, 0.15M NaCl, 10 mM HEPES, pH 7.2) and applied on the sections. Following 10 minutes incubation at RT the sections were washed 5 minutes using 10× diluted S3006 (Dako).

PNA4-dex-HRP (218-021) was diluted to final concentration of 0.05 μM (dextran) in BAP-HEPES-buffer (1.5% BSA, 3% PEG, 0.15M NaCl, 0.05% 4-aminoantipyrin, 10 mM HEPES, pH 7.2) and applied on the sections. Following 10 minutes incubation at RT the sections were washed 5 minutes using 10× diluted S3006 (Dako).

Permanent Red working solution (K0640 Dako) and DAB+ working solution (K3468 Dako) were prepared. The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionized water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: Using detection method 1: CD3=2-layer experiment=AP=2+ specific and 0 non-specific, background staining, MIB-1=3-layer experiment=HRP=1.5+ specific and 0 background staining. Using detection method 2: CD3=2-layer experiment=AP=3+ specific and 1.5+ nonspecific staining, MIB-1=3-layer experiment=HRP=1.5+ specific and 1+ background staining. The order of detection affects the staining result.

### Example 43

### Further Multi-Target Detection Experiment

This example presents a 2-layer detection of two targets in which mouse-Ab-dex-PNA is recognized by PNA-dex-Enzyme1 and rabbit-Ab-dex-PNA is recognized by PNA-dex-Enzyme2. The enzymes are HRP and AP bringing along respectively a brown and red end-product within the same tissue section. As in preceding examples, PNA1 and 2 specifically hybridize, as do PNA3 and 4.

CD3-dex-PNA1 (D16043) and MIB-1-dex-PNA2 (218-097) were both diluted to final concentration of 0.1 μM (dextran) in BP-HEPES-buffer (1.5% BSA, 3% PEG, 0.15M NaCl, 10 mM HEPES, pH 7.2). The two conjugates were applied simultaneously on the sections. Following 10 minutes incubation at RT the sections were washed 5 minutes using 10× diluted S3006 (Dako).

PNA2-dex-HRP (209-141) and PNA4-dex-AP (209-177) were diluted to final concentration of 0.2 μM (dextran)and 0.05 μM (dextran) in BAP-HEPES-buffer (1.5% BSA, 3% PEG, 0.15M NaCl, 0.05% 4-aminoantipyrin, 10 mM HEPES, pH 7.2), respectively. The two conjugates were applied simultaneously on the sections. Following 10 minutes incubation at RT the sections were washed 5 minutes using 10× diluted S3006 (Dako). Permanent Red working solution (K0640 Dako) and DAB+ working solution (K3468 Dako) were prepared.

The reactions were detected with one of the following methods. Detection method 1: Permanent Red working solution was applied. Following 10 minutes incubation the sections were washed 5 minutes using 10× diluted S3006 (Dako). Then DAB+ working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Detection method 2: DAB+ working solution was applied. Following 10 minutes incubation the sections were washed 5 minutes using 10× diluted S3006 (Dako). Then Permanent Red working solution was applied and following 10 minutes incubation the sections were washed 5 minutes using deionized water. Finally the sections were counter stained 5 minutes using haematoxylin S3301 (Dako), rinsed in deionzed water, washed 3 minutes in wash buffer, and mounted in Faramount S3025 (Dako).

Result: Using detection method 1: CD3=HRP=2+ specific and 0 non-specific, background staining, MIB-1=AP=3+ specific and 0 background staining. Using detection method 2: CD3=HRP=2+ specific and 0.5+ background staining, MIB-1=AP=2.5+ specific and 0 background staining. The order of detection affects the staining result.

### Example 44

### 3-Layer Detection System for Detecting MIB-1 Primary Mouse Antibody

Aim: To show that the MIB-1 primary mouse antibody can be detected in a 3-layer system.

Experimental Steps: Primary mouse antibody M7240 (Dako) targeting MIB-1 was diluted to a final 1:150 in S2022 buffer (Dako) and applied on a multi tissue section. Following 10 minutes incubation at RT the section was washed 5 minutes using 10× diluted S3006 (Dako). GaM-dex-PNA1 was diluted to a final concentration of 0.08□M (dextran) in BP-HEPES-buffer (1.5% BSA, 3% PEG, 0.15M NaCl, 10 mM HEPES, pH 7.2) and was applied. Following 10 minutes incubation at RT the section was washed 5 minutes using 10× diluted S3006 buffer (Dako). The sections were rinsed in deionized water.

Following 10 minutes incubation in 0.5% glutaraldehyde at RT the sections were rinsed in deionized water and washed 5 minutes using 10× diluted S3006 (Dako). PNA2-dex-PNA3 was diluted to a final concentration of 0.05□M (dextran) in BP-HEPES-buffer (1.5% BSA, 3% PEG, 0.15M NaCl, 10 mM HEPES, pH 7.2) and was applied. (PNA2 hybridizes to PNA1 while PNA3 hybridizes to PNA4.) Following 10 minutes incubation at RT the section was washed 5 minutes using 10× diluted S3006 (Dako).

PNA4-dex-HRP was diluted to final concentration of 0.05□M (dextran) in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific nuclear MIB-1 staining of proliferating cells. Staining intensity score 2.5+ and background score 1+.

### Example 45

### 2-Layer IHC Detection Test Comparing Orientation of PNA Hybridization

Aim: To use 2-layer HRP detection to test and compare GaM-dex-PNA1+ PNA2-dex-HRP and GaM-dex-PNA2+ PNA1-dex-HRP.

| Unit No. | Unit No. | | |
|---|---|---|---|
| GaM-dex-PNA | PNA-dex-HRP | Specific score | Background score |
| 218-117 | 209-141 | 1.5+ | 0 |
| 218-163 | 218-121 | 0.5+ | 0 |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako). GaM-dex-PNA (218-117 or 218-163) was diluted to final concentration of 0.08□M/dex in BP-HEPES-buffer (1.5% BSA, 3% PEG, 0.15M

NaCl, 10 mM HEPES, pH 7.2) and was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA-dex-HRP (209-141 or 218-121) was diluted to final concentration of 0.05□M/dex in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show that performance depend on which one of the PNA sequences from a PNA pair were used when composing the recognition and detection unit.

### Example 46

### 2-Layer IHC Testing of Recognition Modules with different Linker Length

Aim: to use 2-layer HRP detection to test and compare recognition modules with different linker length (L150, L300, L540).

| Unit No. | Linker length | Specific score | Background score |
|---|---|---|---|
| 209-033 | L150 | 2.5+ | 1+ |
| 209-029 | L300 | 2.5+ | 1+ |
| 195-147 | L540 | 2+ | 0 |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako).

GaM-dex-PNA1 (209-033, 209-029, or 195-147) was diluted to a final concentration of 0.08□M/dex in BP-HEPES-buffer (1.5% BSA, 3% PEG, 0.15M NaCl, 10 mM HEPES, pH 7.2) and was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA2-dex-HRP (209-041) was diluted to final concentration of 0.05□M/dex in BAP-HEPES-buffer (1.5% BSA, 3% PEG, 0.15M NaCl, 0.05% 4-aminoantipyrin, 10 mM HEPES, pH 7.2) and was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show no significant difference in performance when comparing recognition modules having different linker length.

### Example 47

### 2-Layer IHC Testing of Recognition Modules with different Dextran Size

Aim: to use 2-layer HRP detection to test and compare recognition modules with different dextran size (dex70 and dex150).

| Unit No. | Dextran size | Specific score | Background score |
|---|---|---|---|
| 195-147 | Dex70 | 2+ | 0 |
| 195-151 | Dex150 | 2+ | 1+ |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako).

GaM-dex-PNA1 (195-147 or 195-151) was diluted to final concentration of 0.08□M/dex in BP-HEPES-buffer (1.5% BSA, 3% PEG, 0.15M NaCl, 10 mM HEPES, pH 7.2) and was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA2-dex-HRP (209-041) diluted to final concentration of 0.05□M/dex in BAP-HEPES-buffer (1.5% BSA, 3% PEG, 0.15M NaCl, 0.05% 4-aminoantipyrin, 10 mM HEPES, pH 7.2) was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show no significant difference in performance when comparing recognition modules having different dextran size.

### Example 48

### 2-Layer IHC Testing of Detection Modules with different Number of Linker-PNA Attached

Aim: to use 2-layer HRP detection to test and compare detection modules with different number of PNA per dextran (0.8 PNA/dex and 1.5 PNA/dex).

| Unit No. | PNA/dex | Specific score | Background score |
|---|---|---|---|
| 195-051 | 0.8 | 3+ | 0.5+ |
| D15008 | 1.5 | 3+ | 0.5+ |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako).

GaM-dex-PNA1 (195-047) diluted to final concentration of 0.08□M/dex in BP-HEPES-buffer (1.5% BSA, 3% PEG, 0.15M NaCl, 10 mM HEPES, pH 7.2) was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA2-dex-HRP (195-051 or D15008) diluted to final concentration of 0.05□M/dex in BAP-HEPES-buffer (1.5% BSA, 3% PEG, 0.15M NaCl, 0.05% 4-aminoantipyrin, 10 mM HEPES, pH 7.2) was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show no significant difference in performance when comparing detection modules having different number of PNA/dex.

### Example 49

### 2-Layer IHC Testing Comparing Recognition and Detection Modules having “Linker-PNA” or “Linker-PNA-Linker Tail” Attached

Aim: to use 2-layer HRP detection to test and compare recognition and detection modules having PNA sequences without and with “linker tail”.

| Unit No. | | | | | |
|---|---|---|---|---|---|
| GaM-dex- | | Unit No. PNA- | | Specific | Background |
| PNA | tail | dex-HRP | tail | score | score |
| 218-113 | No | 218-021 | No | 3+ | 1+ |
| D16074 | Yes | 218-021 | No | 3+ | 0.5+ |
| 218-113 | No | D16076 | Yes | 3+ | 0.5+ |
| D16074 | Yes | D16076 | Yes | 4+ | 1.5+ |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako). GaM-dex-PNA (218-113 or D16074) diluted to final concentration of 0.08□M/dex in BP-HEPES-buffer (1.5% BSA, 3% PEG, 0.15M NaCl, 10 mM HEPES, pH 7.2) was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA-dex-HRP (218-021 or D16076) diluted to final concentration of 0.05 μM/dex in BAP-HEPES-buffer (1.5% BSA, 3% PEG, 0.15M NaCl, 0.05% 4-aminoantipyrin, 10 mM HEPES, pH 7.2) was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show that performance depend on the presence of a “linker tail” on the PNA sequence. A “linker-tail” on the PNA sequence may influence both specific score and background score.

### Example 50

### 2-Layer IHC Testing Comparing Recognition and Detection Modules having “Linker-PNA” or “Linker-PNA-Charge” Attached

Aim: to use 2-layer HRP detection to test and compare recognition and detection modules having PNA sequences without and with charge.

| Unit No. | | Unit No. | | | |
|---|---|---|---|---|---|
| GaM-dex- | | PNA-dex- | | | Background |
| PNA | charge | HRP | charge | Specific score | score |
| D15078 | No | D15069 | No | 0.5+ | 0 |
| 209-149 | Yes | 209-157 | Yes | 3+ | 1+ |

Experimental Steps: Primary mouse antibody M3515 (Dako) targeting CK was diluted to final 1:200 in S2022 (Dako) and applied on a multi tissue section. Following 10 minutes incubation at RT the section was washed 5 minutes using 10× diluted S3006 (Dako).

GaM-dex-PNA (D15078 or 209-149) diluted to final concentration of 0.08 μM/dex in BP-HEPES-buffer (1.5% BSA, 3% PEG, 0.15M NaCl, 10 mM HEPES, pH 7.2) was applied. Following 10 minutes incubation at RT the section was washed 5 minutes using 10× diluted S3006 (Dako). The sections were rinsed in deionized water. Following 10 minutes incubation in 0.5% glutaraldehyde at RT the sections were rinsed in deionized water and washed 5 minutes using 10× diluted S3006 (Dako).

PNA-dex-HRP (D15069 or 209-157) diluted to final concentration of 0.05□M/dex in BAP-HEPES-buffer (1.5% BSA, 3% PEG, 0.15M NaCl, 0.05% 4-aminoantipyrin, 10 mM HEPES, pH 7.2) was applied. Following 10 minutes incubation at RT the sections were washed 5 minutes using 10× diluted S3006 (Dako).

Prepared DAB+ working solution (Dako K3468) was applied. Following 10 minutes incubation the sections were washed 5 minutes deionized water. Finally the sections were counter stained 5 minutes using haematoxylin S3301 (Dako), rinsed in deionzed water, washed 3 minutes in wash buffer, and mounted in Faramount S3025 (Dako).

Result: The brown end product of the HRP reaction visualize the specific cytokeratin staining of epithelia cells and show that performance depend on the presence of charge on the PNA sequence. Charge on the PNA sequences within a PNA pair may influence both specific score and background score.

### Example 51

### Method of Synthesizing Mono and 2,4-diamino-pyrimidine-5-yl PNA Monomers

2,4-diamino-pyrimidine-5-yl may be introduced into DNA-oligomers by methods known in the art (e.g. S. A. Benner et al., Nucleic Acid Research 24(7): 1308-1313 (1996)). A corresponding PNA oligomer is prepared by chlorinating pyrimidine-5-acetic acid to yield 2-chloro-pyrimidine-5-acetic acid, 4-chloro-pyrimidine-5-acetic acid, and 2,4-dichloro-pyrimidine-5-acetic acid. Separation of isomers, followed by high temperature and pressure treatment with ammonia, gives the three corresponding amino-pyrimidine derivatives (see FIG. 9). The amino-pyrimidine derivatives are separated and amino-protected, then coupled to a protected PNA backbone ester. Ester hydrolysis results in PNA monomers for production of PNA oligomers containing 2-amino; 4-amino; and/or 2,4-diamino pyrimidine-5-yl bases.

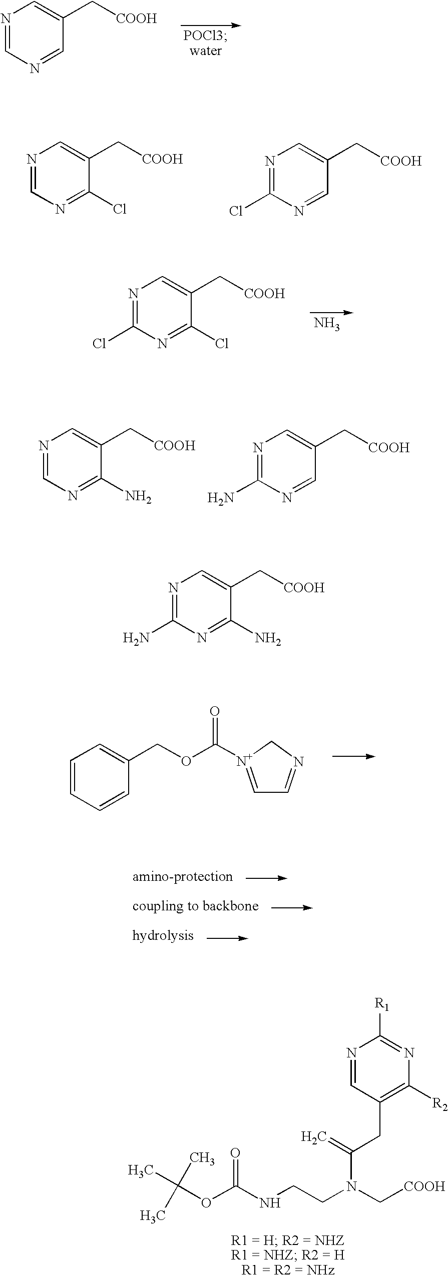

### Example 52

### Synthesis of Xanthine and Thio-Xanthine-Coupled PNA Monomers

Xanthine, 2-thio-xanthine, and 6-thio-xanthine are commercially available, for instance, from ScienceLab.com. Further, S. A. Benner et al., Nucleic Acid Research 24(7): 1308-1313 (1996) teaches the preparation of a xanthosine-DNA monomer, including a less acidic and preferable 7-deaza analog, and notes the preferred protection of both oxygens during solid phase synthesis.

Xanthine PNA-monomers, as well as 2-thio and 6-thio xanthine monomers, are prepared by:
1. Protecting both oxygens or both oxygen and sulphur with appropriate protection groups such as (possibly substituted) benzyl.
2. Alkylating at N-9 with ethyl bromacetate. (Separating N-7 alkylated byproduct.)
3. Hydrolyzing the ethyl ester.
4. HBTU or Carbodiimide-mediated coupling of the nucleobase-acids to 2-Boc-aminoethyl-ethylglycinate.
5. Hydrolyzing the resulting monomer ester to the monomer free acid.
6. The resulting monomers may be used in Merrifield solid phase synthesis of xanthine, 2-thio-xanthine and 6-thio-xanthine-containing PNAs.

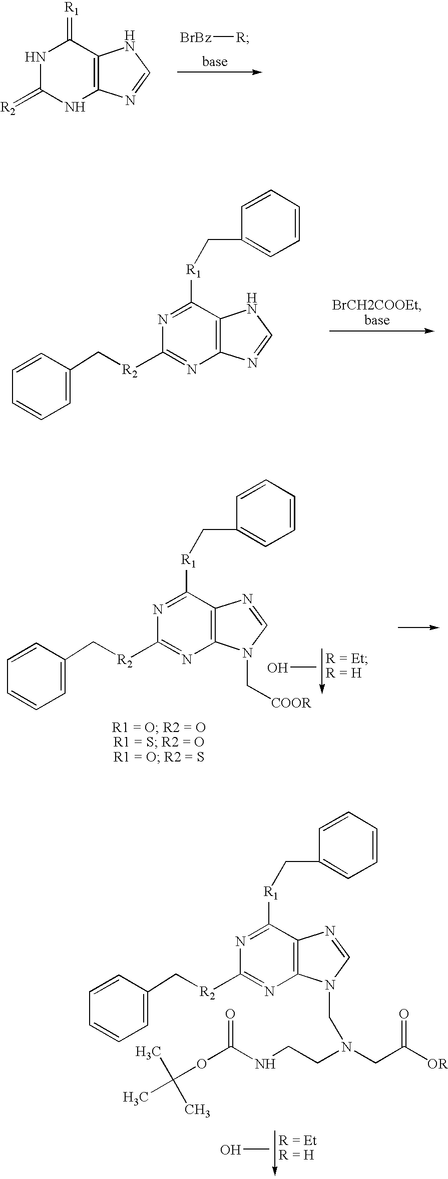

Other embodiments of the invention will be apparent to those skilled in the art from consideration of the specification and practice of the invention disclosed herein. It is intended that the specification and examples be considered as exemplary only.

What is claimed is:

1. An automated method of detecting at least one target in a sample, comprising:
a) obtaining a sample comprising at least one target;
b) contacting the sample with at least one probe specific for the at least one target;
c) contacting the sample with at least one detectable label; and
d) detecting the presence of the at least one target with the at least one detectable label;
wherein, following one or more of parts (a)-(c), the sample is incubated with at least one cross-linking agent; and wherein progress from one or more of parts (a)-(d) is automatically controlled.

2. The method of claim 1, wherein the sample is incubated with the at least one cross-linking agent between parts (b) and (c).

3. The method of claim 2, wherein, following addition of the cross-linking agent between parts (b) and (c), buffer conditions of the sample are changed to conditions that without addition of the cross-linking agent would significantly reduce signal generated from the first target.

4. The method of claim 1, wherein the sample is incubated with the at least one cross-linking agent following parts (c) and (d).

5. The method of claim 4, wherein, following addition of the cross-linking agent between parts (c) and (d), buffer conditions of the sample are changed to conditions that without addition of the cross-linking agent would significantly reduce signal from either or both of the at least one first target and at least one second target.

6. The method of claim 1, wherein the at least one first target and the at least one second target comprise nucleic acids.

7. The method of claim 1, wherein the at least one first target and the at least one second target comprise proteins.

8. The method of claim 1, wherein the at least one first target is a protein and the at least one second target is a nucleic acid.

9. The method of claim 1, wherein the at least one first target is a nucleic acid and the at least one second target is a protein.

10. The method of claim 1, wherein the cross-linking agent covalently attaches the sample to at least one carrier.

11. The method of claim 1, wherein the at least one first probe and/or at least one second probe is an antibody.

12. The method of claim 1, wherein the at least one first probe and/or at least one second probe is a nucleic acid.

13. The method of claim 12, wherein the nucleic acid is a nucleic acid analog.

14. The method of claim 1, comprising at least one first detectable label to detect the at least one first target and at least one second detectable label to detect the at least one second target, wherein the at least one first detectable label and the at least one second detectable label are distinguishable from each other.

15. An automated method of detecting a target in a sample, comprising:
a) obtaining a sample comprising at least one first target and at least one second target;
b) contacting the sample with at least one first probe specific for the at least one first target;
c) contacting the sample with at least one second probe specific for the at least one second target;
d) contacting the sample with at least one detectable label; and
e) detecting the presence of the at least one first target and at least one second target with the at least one detectable label;
wherein, following one or more of parts (a)-(d), the sample is incubated with at least one cross-linking agent; and wherein progress from one or more of parts (a)-(e) is automatically controlled.

16. The method of claim 15, wherein the sample is incubated with the at least one cross-linking agent between parts (b) and (c).

17. The method of claim 16, wherein, following addition of the cross-linking agent between parts (b) and (c), buffer conditions of the sample are changed to conditions that without addition of the cross-linking agent would significantly reduce signal generated from the first target.

18. The method of claim 15, wherein the sample is incubated with the at least one cross-linking agent following parts (c) and (d).

19. The method of claim 18, wherein, following addition of the cross-linking agent between parts (c) and (d), buffer conditions of the sample are changed to conditions that without addition of the cross-linking agent would significantly reduce signal from either or both of the at least one first target and at least one second target.

20. The method of claim 15, wherein the sample is incubated with the at least one cross-linking agent between parts (d) and (e).

21. The method of claim 20, wherein, following addition of the cross-linking agent between parts (c) and (d), buffer conditions of the sample are changed to conditions that without addition of the cross-linking agent would significantly reduce signal from either or both of the at least one first target and at least one second target.

22. The method of claim 15, wherein the at least one first target and the at least one second target comprise nucleic acids.

23. The method of claim 15, wherein the at least one first target and the at least one second target comprise proteins.

24. The method of claim 15, wherein the at least one first target is a protein and the at least one second target is a nucleic acid.

25. The method of claim 15, wherein the at least one first target is a nucleic acid and the at least one second target is a protein.

26. The method of claim 15, wherein the cross-linking agent covalently attaches the sample to at least one carrier.

27. The method of claim 15, wherein the at least one first probe and/or at least one second probe is an antibody.

28. The method of claim 15, wherein the at least one first probe and/or at least one second probe is a nucleic acid.

29. The method of claim 28, wherein the nucleic acid is a nucleic acid analog.

30. The method of claim 15, comprising at least one first detectable label to detect the at least one first target and at least one second detectable label to detect the at least one second target, wherein the at least one first detectable label and the at least one second detectable label are distinguishable from each other.

31. An automated method of detecting at least two targets in a sample, comprising:
a) obtaining a sample comprising at least one first target and at least one second target;
b) contacting the sample with at least one first probe specific for the at least one first target;
c) contacting the sample with at least one second probe specific for the at least one second target;
d) adding at least one adaptor unit specific for the at least one first probe and/or for the at least one second probe;
e) adding at least one detectable label specific for the at least one adaptor unit; and
f) detecting the presence of the at least one first target and at least one second target with the at least one detectable label;
wherein, following one or more of parts (a)-(d), the sample is incubated with at least one cross-linking agent; and wherein progress from one or more of parts (a)-(f) is automatically controlled.

32. The method of claim 31, wherein the at least one adaptor unit amplifies signal from the at least one detectable label.

33. The method of claim 31, wherein the sample is incubated with the at least one cross-linking agent between parts (b) and (c).

34. The method of claim 33, wherein, following addition of the cross-linking agent between parts (c) and (d), buffer conditions of the sample are changed to conditions that without addition of the cross-linking agent would significantly reduce signal from the at least one first target.

35. The method of claim 31, wherein the sample is incubated with the at least one cross-linking agent following parts (c) and (d).

36. The method of claim 35, wherein, following addition of the cross-linking agent between parts (c) and (d), buffer conditions of the sample are changed to conditions that without addition of the cross-linking agent would significantly reduce signal from either or both of the at least one first target and at least one second target.

37. The method of claim 31, wherein the sample is incubated with the at least one cross-linking agent between parts (d) and (e).

38. The method of claim 37, wherein, following addition of the cross-linking agent between parts (c) and (d), buffer conditions of the sample are changed to conditions that without addition of the cross-linking agent would significantly reduce signal from either or both of the at least one first target and at least one second target.

39. The method of claim 31, wherein the sample is incubated with the at least one cross-linking agent between parts (e) and (f).

40. The method of claim 39, wherein, following addition of the cross-linking agent between parts (c) and (d), buffer conditions of the sample are changed to conditions that without addition of the cross-linking agent would significantly reduce signal from either or both of the at least one first target and at least one second target.

41. The method of claim 31, wherein the at least one first target and the at least one second target comprise nucleic acids.

42. The method of claim 31, wherein the at least one first target and the at least one second target comprise proteins.

43. The method of claim 31, wherein the at least one first target is a protein and the at least one second target is a nucleic acid.

44. The method of claim 31, wherein the at least one first target is a nucleic acid and the at least one second target is a protein.

45. The method of claim 31, wherein the cross-linking agent covalently attaches the sample to at least one carrier.

46. The method of claim 31, wherein the at least one first probe and/or at least one second probe is an antibody.

47. The method of claim 31, wherein the at least one first probe and/or at least one second probe is a nucleic acid.

48. The method of claim 47, wherein the nucleic acid is a nucleic acid analog.

49. The method of claim 17, wherein the cross-linking agent covalently attaches the sample to at least one carrier.

50. The method of claim 31, comprising at least one first detectable label to detect the at least one first target and at least one second detectable label to detect the at least one second target, wherein the at least one first detectable label and the at least one second detectable label are distinguishable from each other.

51. A kit for carrying out a detection method, comprising:
a) one or more containers comprising a first probe and a first detectable label for detecting a first target in a sample and associated buffers;
b) optionally, one or more containers comprising a second probe and a second detectable label for detecting a first target in a sample and associated buffers; and
c) one or more containers comprising one or more cross-linking agents.

52. The kit of claim 37, further comprising one or more containers comprising one or more adaptor units specific for the at least one first probe and/or the at least one second probe and associated buffers.

53. The kit of claim 37 wherein the at least one first probe and/or at least one second probe is a nucleic acid.

54. The kit of claim 38 wherein the nucleic acid is a nucleic acid analog.

55. The kit of claim 37, wherein the at least one first probe and/or at least one second probe is an antibody.

56. The kit of claim 37, comprising at least one first detectable label for the at least one first probe and at least one second detectable label for the at least one second probe, wherein the at least one first detectable label and at least one second detectable label are distinguishable from each other.


SEQUENCE LISTING (22 sequences)

SEQ ID NO: 1 - DNA, 10 bp, Artificial Sequence
  Description of Artificial Sequence Synthetic PNA sequence
tcnnnntaca

SEQ ID NO: 2 - DNA, 10 bp, Artificial Sequence
  Description of Combined DNA/RNA Molecule Synthetic PNA sequence
ngnnnnttgn

SEQ ID NO: 3 - DNA, 12 bp, Artificial Sequence
  Description of Combined DNA/RNA Molecule Synthetic PNA sequence
cnnnnntnnnnc

SEQ ID NO: 4 - DNA, 12 bp, Artificial Sequence
  Description of Artificial Sequence Synthetic PNA sequence
gtntaattnnag

SEQ ID NO: 5 - DNA, 12 bp, Artificial Sequence
  Description of Combined DNA/RNA Molecule Synthetic PNA sequence
nntcnnnngncn

SEQ ID NO: 6 - DNA, 12 bp, Artificial Sequence
  Description of Artificial Sequence Synthetic PNA sequence
agacnttngant

SEQ ID NO: 7 - DNA, 10 bp, Artificial Sequence
  Description of Artificial Sequence Synthetic PNA sequence
tcnnnntaca

SEQ ID NO: 8 - DNA, 12 bp, Artificial Sequence
  Description of Artificial Sequence Synthetic PNA sequence
ngtntcgtnccg

SEQ ID NO: 9 - DNA, 18 bp, Artificial Sequence
  Description of Artificial Sequence Synthetic PNA sequence
aacgggataactgcacct

SEQ ID NO: 10 - DNA, 10 bp, Artificial Sequence
  Description of Artificial Sequence Synthetic PNA sequence
tcaaggtaca

SEQ ID NO: 11 - DNA, 10 bp, Artificial Sequence
  Description of Artificial Sequence Synthetic PNA sequence
tgtaccttga

SEQ ID NO: 12 - DNA, 12 bp, Artificial Sequence
  Description of Artificial Sequence Synthetic PNA sequence
agacnttngnnt

SEQ ID NO: 13 - DNA, 12 bp, Artificial Sequence
  Description of Artificial Sequence Synthetic PNA sequence
gtntanttnnag

SEQ ID NO: 14 - DNA, 10 bp, Artificial Sequence
  Description of Artificial Sequence Synthetic PNA sequence
ttgannttag

SEQ ID NO: 15 - DNA, 10 bp, Artificial Sequence
  Description of Artificial Sequence Synthetic PNA sequence
tgtannttga

SEQ ID NO: 16 - DNA, 10 bp, Artificial Sequence
  Description of Combined DNA/RNA Molecule Synthetic PNA sequence
ngnnnnttgn

SEQ ID NO: 17 - DNA, 12 bp, Artificial Sequence
  Description of Combined DNA/RNA Molecule Synthetic PNA sequence
ngtcnnnngncn

SEQ ID NO: 18 - DNA, 12 bp, Artificial Sequence
  Description of Combined DNA/RNA Molecule Synthetic PNA sequence
ncnnnntnngnc

SEQ ID NO: 19 - DNA, 10 bp, Artificial Sequence
  Description of Artificial Sequence Synthetic PNA sequence
ctaanntcaa

SEQ ID NO: 20 - DNA, 10 bp, Artificial Sequence
  Description of Artificial Sequence Synthetic PNA sequence
tcaanntaca

SEQ ID NO: 21 - DNA, 10 bp, Artificial Sequence
  Description of Artificial Sequence Synthetic PNA sequence
ctaaggtcaa

SEQ ID NO: 22 - DNA, 10 bp, Artificial Sequence
  Description of Artificial Sequence Synthetic PNA sequence
tcnnggtaca